(19) United States
(12) Patent Application Publication
(10) Pub. No.: US 2023/0391792 A1
(43) Pub. Date: Dec. 7, 2023

(54) METHODS AND COMPOSITIONS FOR TARGETED PROTEIN DEGRADATION

(71) Applicant: Ranok Therapeutics (Hangzhou) Co. Ltd., Hangzhou (CN)

(72) Inventors: Weiwen Ying, Lexington, MA (US); Kevin P. Foley, Waltham, MA (US); Long Ye, Shanghai (CN); Mingkai Wang, Hangzhou (CN); Chenghao Ying, Shanghai (CN); Wei Yin, Lianyungang (CN); Lingjie Zhang, Wuxi (CN)

(21) Appl. No.: 18/031,701

(22) PCT Filed: Oct. 14, 2021
(86) PCT No.: PCT/CN2021/123935

§ 371 Date: Apr. 13, 2023

(30) Foreign Application Priority Data
Oct. 14, 2020 (WO) PCT/CN2020/120945

(51) Int. Cl. C07D 495/14; C07D 487/04; C07D 471/04; C07D 403/14; A61P 35/00

(52) U.S. Cl. CPC C07D 495/14; C07D 487/04; C07D 471/04; C07D 403/14; A61P 35/00

(57) ABSTRACT

Provided are compounds of Formula: H-L-T and pharmaceutically acceptable salts and compositions thereof, which are useful for treating cancers and related conditions.

### BACKGROUND

Protein homeostasis, or proteostasis, refers to the ability of cells to regulate the synthesis, folding, trafficking and degradation of proteins. In particular, properly regulated protein degradation is required for the normal functioning of cells, including their proliferation, differentiation and death, and is often dysregulated in cancers and other diseases (Van Die, Chin J Cancer, 2011, 30:124-137).

The ubiquitin-proteasome system (UPS) is one of the major pathways in cells that mediates the disposal and metabolic recycling of proteins (Yu and Matouschek, Annu Rev Biophys, 2017, 46:149-173; Navon and Ciechanover, J Biol Chem, 2009, 284:33713-33718). Ubiquitin is a 76 amino acid-residue protein that is ubiquitously expressed. With respect to protein degradation by the UPS, the process of ubiquitination occurs when a ubiquitin is attached to a lysine amino acid residue in a substrate protein, which involves a series of enzymatic steps. First, ubiquitin is transferred to an E1 ubiquitin-activating enzyme. Second, activated ubiquitin is transferred from the E1 to an E2 ubiquitin-conjugating enzyme. And third, one of the several hundred different E3 ubiquitin ligase enzymes links the ubiquitin to a lysine residue in a substrate protein. Repetition of this enzymatic process results in tagging substrate proteins with polyubiquitin chains. Such ubiquitin-tagged proteins can then be delivered to the proteasome, a large multi-subunit complex that degrades proteins. The ability of some cellular chaperone proteins and chaperone complexes to direct proteins towards the UPS is facilitated by their direct interaction with E3 ubiquitin ligases (Amm et al., Biochim Biophys Acta, 2014, 1843:182-196; Taipale et al., Cell, 2012, 150:987-1001). In addition to protein degradation, the ubiquitination of proteins can also regulate other processes, such as subcellular localization, activity and protein-protein interactions.

Chemically induced, targeted protein degradation (TPD) has emerged as a new modality for small molecule drug development. A small molecule can be used to promote the interaction of a target protein or proteins with a component or components of various cellular protein degradation pathways, thereby inducing the degradation of the targeted protein or proteins as a way to treat disease.

In particular, proteolysis-targeting chimeras (PROTACs) are an example of such small molecules that purposely induce protein degradation of specific proteins by coopting the UPS (Burslem and Crews, Cell, 2020, 181:102-114; Pettersson and Crews, Drug Discov Today Technol, 2019, 31:15-27). PROTAC molecules are bifunctional small molecules that simultaneously bind to a target protein or proteins and an E3 ubiquitin ligase, creating ternary complexes in cells between the target protein(s), the PROTAC molecule and an E3 ligase protein. The induced proximity of the target protein(s) and the E3 ligase causes the ubiquitination of the target protein(s) and subsequent degradation of the target protein(s) by the proteasome. Although PROTACs that incorporate target protein binders that promiscuously bind to multiple proteins can often degrade multiple proteins, in some cases protein-protein interactions between individual targets and an E3 ligase can increase or decrease the observed potency and selectivity of degradation, for example by inhibiting formation of some ternary complexes due to charge repulsion and steric clashing between a given target protein and E3 ligase pair (Pettersson and Crews, Drug Discov Today Technol, 2019, 31:15-27; Bondeson et al., Cell Chem Biol, 2018, 25:78-87; Gadd et al., Nat Chem Biol, 2017, 13:514-521; Zengerle et al., ACS Chem Biol, 2015, 10:1770-1777).

Other methods to chemically induce TPD have also been described, such as molecular glues (Che et al., Bioog Med Chem Lett, 2018, 28:2585-2592), AUTACs, ATTECs and LYTACs (Ding et al., Trends Pharmacol Sci, 2020, 41:464-474). For example, AUTAC technology follows a similar principle of induced proximity, but targets proteins for degradation via autophagy (Daiki et al., Mol Cell, 2019, 76:797-810).

Collectively, TPD technologies have a number of advantages over conventional biochemical inhibitors (Pettersson and Crews, Drug Discov Today Technol, 2019, 31:15-27; Ding et al., Trends Pharmacol Sci, 2020, 41:464-474). For example, unlike conventional inhibitors, TPD agents work sub-stoichiometrically and can typically mediate the sequential degradation of multiple molecules of the target protein(s), often leading to greater potency than the isolated target binding moiety that they incorporate and other biochemical inhibitors. Also, since inhibition of target protein(s) function by TPD agents is principally due to degradation rather than solely biochemical inhibition, recovery of the function of target protein(s) is typically slower than is observed for biochemical inhibitors. TPD agents may also have improved target selectivity over biochemical inhibitors. Finally, TPD agents can target proteins that are not amenable to biochemical inhibition by interacting with binding pockets that do not affect the biochemical activity of the target but still permit its degradation.

However, some disadvantages are associated with current TPD technologies. These include the promiscuous degradation of the target protein(s) in many tissues and organs, not just the tissue(s) and organ(s) where the target protein(s) is involved in a disease process, which is expected to result in unwanted side effects of treatment. Also, resistance to these technologies can develop through mutations or alterations in expression of components of the UPS such as E3 ligases (Ottis et al., ACS Chem Biol, 2019, 14:2215-2223; Zhang et al., Mol Cancer Ther, 2019, 18:1302-1311), resulting in loss of therapeutic efficacy. As such, a need exists for improved/alternative methods and compositions for TPD. It is also desirable to develop improved/alternative TPD agents that mediate the degradation of proteins involved in cancer and other diseases.

### SUMMARY

Provided herein are compounds which comprise three components: 1) a chemical moiety capable of binding a target protein or proteins; 2) a chemical moiety capable of binding a chaperone protein or proteins or component of a chaperone complex; and 3) a chemical moiety (linker) that joins the other two other moieties.

Also provided are precursors for forming such compounds, wherein said precursors are comprised of 1) a chemical moiety capable of binding a chaperone protein or proteins or component of a chaperone complex (e.g., HSP90); and 2) a chemical moiety (linker).

### DETAILED DESCRIPTION

### 1. General Description of Compounds

Provided herein are compounds having the Formula H-L-T, wherein H is a chemical moiety capable of binding a target protein or proteins; 2) a chemical moiety capable of binding a chaperone protein or proteins or component of a chaperone complex (e.g., HSP90, KRAS, MAPK7); and 3) a chemical moiety (linker) that joins the other two other moieties.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” are used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkenyl” means a saturated straight chain or branched non-cyclic hydrocarbon having, unless specified otherwise, from 2 to 10 carbon atoms (e.g., (C₂-C₆)alkenyl or (C₂-C₄)alkenyl) and having at least one carbon-carbon double bond. Representative straight chain and branched (C₂ -C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from e.g., 3 to 10 carbon atoms (e.g., from 4 to 6 carbon atoms). Representative cycloalkyls include cyclopropyl, cyclobutyl, cyclopentyl, cyclohexyl, cycloheptyl, cyclooctyl, cyclononyl, and cyclodecanyl.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker.

As used herein, an “haloalkoxy” is an haloalkyl group which is attached to another moiety via an oxygen linker.

As used herein, the term “alkylene” refers to an alkyl group that has two points of attachment. Straight chain alkylene groups are preferred. Non-limiting examples of alkylene groups include methylene ethylene, n-propylene, isopropylene, and the like. Alkylene groups may be optionally substituted with one or more substituents.

As used herein, the term “heterocyclyl” means a monocyclic heterocyclic ring system which is either a saturated ring or an unsaturated non-aromatic ring comprising, as size and valency permits, up to 5 heteroatoms independently selected from nitrogen, oxygen, and sulfur. The heterocycle may be attached via any heteroatom or carbon atom. Representative heterocycles include morpholinyl, thiomorpholinyl, pyrrolidinonyl, pyrrolidinyl, piperidinyl, piperazinyl, oxiranyl, oxetanyl, tetrahydrofuranyl, tetrahydropyranyl, tetrahydropyrindinyl, tetrahydropyrimidinyl, and the like.

As used herein, the term “heteroaryl” means, as the defined size permits, a monocyclic or polycyclic heteroaromatic ring comprising carbon atom ring members and one or more heteroatom ring members selected from nitrogen, oxygen, and sulfur. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. The point of attachment of a heteroaromatic or heteroaryl ring to another group may be at either a carbon atom or a heteroatom of the heteroaromatic or heteroaryl rings.

As used herein, the term “halogen” or “halo” means F, Cl, Br or I.

The term “oxo” means the group ═O.

When a heterocyclyl or heteroaryl, group contains a nitrogen atom, it may be substituted or unsubstituted as valency permits.

The term “linker” or “tether,” used interchangeably, refers to a chemical moiety that joins two other moieties (e.g., a first binding moiety and a second binding moiety). A linker can covalently join a first binding moiety and a second binding moiety. In one aspect, the linker is uncleavable in vivo. In one aspect, the linker comprises one or more cyclic ring systems. In another aspect, the linker comprises an alkyl chain optionally substituted by and/or interrupted with one or more chemical groups. In one aspect, the linker comprises optimal spatial and chemical properties to effectuate optimal therapeutic activity. In one aspect, the linker does not interfere with the ability of the first binding moiety and/or the second binding moiety to bind their respective targets (e.g., HSP90 and the protein(s) targeted for degradation, such as KRAS or MAPK7). In one aspect, the linker alters the ability of the first binding moiety and/or the second binding moiety to bind their respective targets (e.g., HSP90 and the protein(s) targeted for degradation, such as KRAS or MAPK7).

The term “MAPK7” refers to the protein product of the mitogen-activated protein kinase 7 gene, also known as the extracellular-signal-regulated kinase 5 or ERK5 gene.

The term “KRAS” refers collectively, individually or in various combinations to the protein product of the wild type or mutated KRAS proto-oncogene, GTPase gene.

The term “HSP70” refers collectively, individually or in various combinations to the protein products of members of the heat shock protein family A (70 kDa) gene family, including but not limited to: HSPA1A (HSP70-1), HSPA1B (HSP70-2), HSPA 1L (HSP70-HOM) and HSPA8 (HSC70).

The term “HSP90” refers collectively, individually or in various combinations to the protein products of members of the heat shock protein 90 (90 kDa) gene family, including: HSP9OAA1 (HSP90-alpha or HSP90α), HSP90AB1 (HSP90-beta or HSP90(3), HSP90B1 (GRP94) and TRAP1.

When used in connection to describe a chemical group that may have multiple points of attachment, a hyphen (-) designates the point of attachment of that group to the variable to which it is defined. For example, —NR^{a}R^{b} and —C(O)NR^{a}(C₁₋₄alkylene)NR^{a}R mean that the point of attachment for these groups occur on the nitrogen atom and carbon atom respectively.

A hash bond as in “ [symbol] ” represents the point at which the depicted group is attached to the defined variable.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. For example, when a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. In another preferred embodiment, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

In a first embodiment, provided is a compound of the Formula H-L-T or a pharmaceutically acceptable salt thereof, wherein H is an HSP90, KRAS, or ERK5 binder; L is a linker; and T is a target protein binder.

In a second embodiment, H in the compounds of H-L-T is selected from

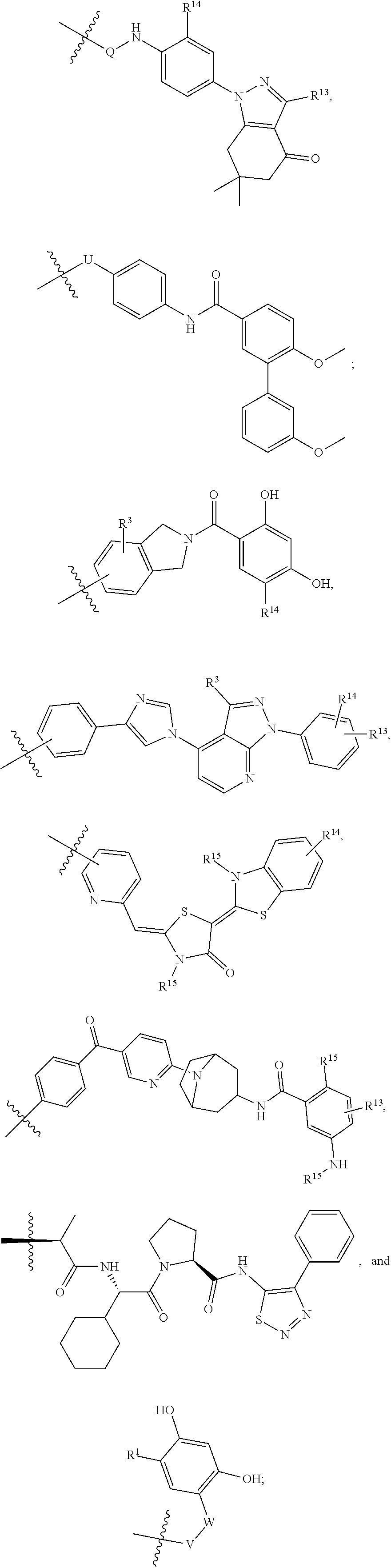

wherein
Q and U are each independently selected from phenyl, heteroaryl, heterocyclyl, and cycloalkyl, each of which being optionally substituted with 1 to 3 groups selected from R²;
R¹³ and R¹⁴ are each independently selected from hydrogen, halo, —CN, (C₁-C₄)alkyl, halo(C₁-C₄)alkyl, and —C(O)NR^{a}R^{b};
R¹⁵ is hydrogen, (C₁-C₄)alkyl, or halo(C₁-C₄)alkyl;
W is 5- or 6-membered heteroaryl optionally substituted with 1 to 3 groups selected from R²;
V is phenyl or 5- to 9-membered heteroaryl optionally substituted with 1 to 3 groups selected from R³;
R¹ is halo, (C₁-C₄)alkyl, halo(C₁-C₄)alkyl, (C₁-C₄)alkoxy, or halo(C₁-C₄)alkoxy;
R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄alkylene)NR^{a}R^{b}, —C₁₋₄alkylNR^{a}R^{b}, —SR^{a}, —S(O)R^{a}, —S(O)₂R^{a}, —S(O)NR^{a}R^{b}, —SO₂NR^{a}R^{b}, —NR^{a}(C₁₋₄alkyl)OR^{a}, —SH, —S(C₁₋₄alkyl), —NR^{a}(C₁₋₄alkyl)NR^{a}R^{b}, —C₁₋₆alkylC(O)NR^{a}R^{b}, —O(C₁₋₄alkylene)NR^{a}C(O)(C₁₋₄alkylene)NR^{a}R^{b}, phenyl or 5- to 7-membered heteroaryl, wherein said phenyl and 5- to 7-membered heteroaryl are each optionally and independently substituted with 1 to 3 groups selected from R⁴;
R^{a} and R^{b} are each independently selected from hydrogen and (C₁-C₄)alkyl, wherein said (C₁-C₄)alkyl is optionally substituted with one or more halo or a 3- to 7-membered heterocyclyl, or both; and
R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy; and wherein the remaining features are described above for H-L-T. Alternatively, as part of a second embodiment, H is

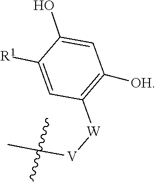

wherein the remaining features are described above. Alternatively, as part of a second embodiment, H is selected from

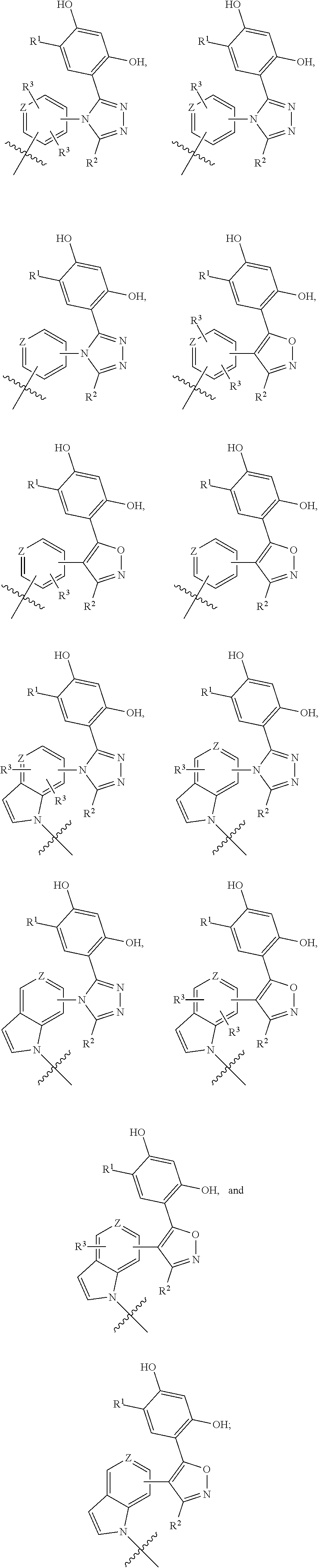

and
Z is N or CH, wherein the remaining features are as described above. In one aspect of this second embodiment, Z is CH.

In a third embodiment, R³ is independently (C₁-C₄)alkyl or halo, wherein the remaining features are as described above for H-L-T or the second embodiment.

In a fourth embodiment, H is

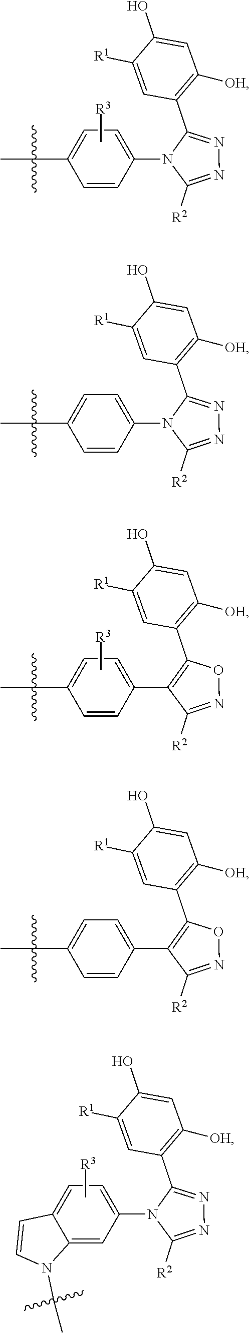

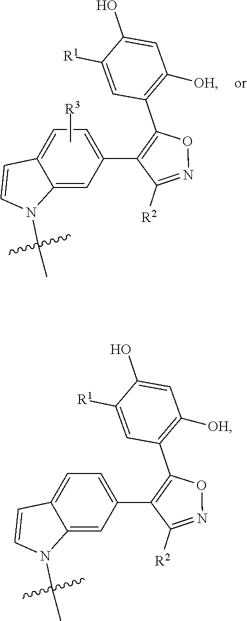

wherein the remaining features are as described above for H-L-T or the second embodiment. Alternatively, as part of a fourth embodiment, H is

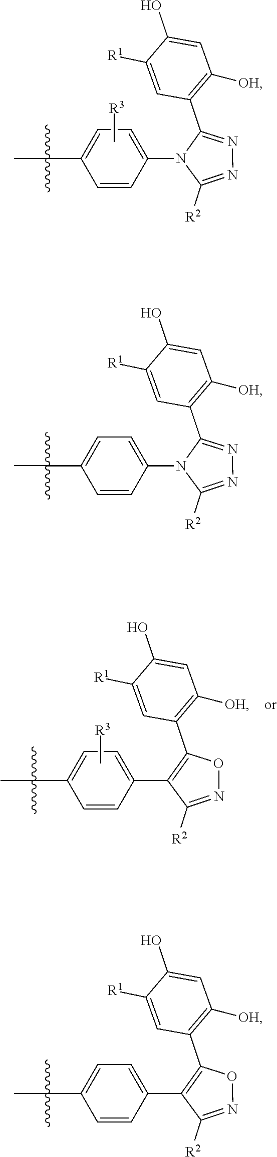

wherein the remaining features are as described above for H-L-T or the second embodiment. Alternatively, as part of a fourth embodiment, H is

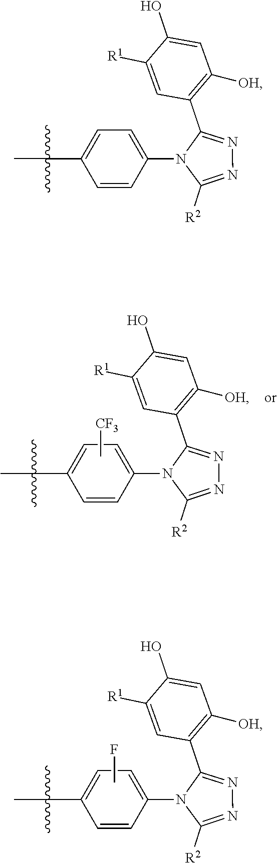

wherein the remaining features are as described above for H-L-T or the second embodiment.
Alternatively, as part of a fourth embodiment, H is

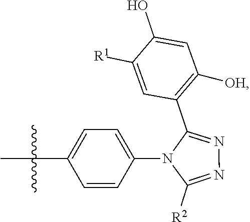

wherein the remaining features are as described above for H-L-T or the second embodiment.

In a fifth embodiment, R¹ is halo or (C₁-C₄)alkyl, wherein the remaining features are as described above for H-L-T or the second, third, or fourth embodiment. Alternatively, as part of a fifth embodiment, R¹ is chloro, isopropyl, methyl, propyl, or ethyl, wherein the remaining features are as described above for H-L-T or the second, third, or fourth embodiment. Alternatively, as part of a fifth embodiment, R¹ is isopropyl or ethyl, wherein the remaining features are as described above for H-L-T or the second, third, or fourth embodiment.

In a sixth embodiment, R² is —OR^{a}, —SR^{a}, —C(O)NR^{a}R^{b}, or —C(O)NR^{a}(C₁₋₄alkylene)NR^{a}R^{b}, wherein the remaining features are as described above for H-L-T or the second, third, fourth, or fifth embodiment.

In a seventh embodiment, R^{a} and R^{b} are each independently selected from hydrogen and (C₁-C₄)alkyl, wherein said (C₁-C₄)alkyl is optionally substituted with 1 to 3 halo or a 6-membered heterocyclyl, wherein the remaining features are as described above for H-L-T or the second, third, fourth, fifth, or sixth embodiment.

In an eighth embodiment, R² is OH, —C(O)NHCH₂CF₃, —C(O)NHCH₂CH₃, —C(O)NHCH(CH₃)₂, —C(O)NH(CH₂CH₃)₂, —C(O)NHCH(CH₃)CF₃, —C(O)NHcyclopropyl, —C(O)NHmethylcyclopropyl, C(O)NH₂, or —C(O)NH(CH₂)₂ piperidinyl, wherein the remaining features are as described above for H-L-T or the second, third, fourth, fifth, sixth, or seventh embodiment. Alternatively, as part of an eighth embodiment, R² is —C(O)NHCH₂CF₃ or OH, wherein the remaining features are as described above for H-L-T or the second, third, fourth, fifth, sixth, or seventh embodiment. Alternatively, as part of an eighth embodiment, R² is OH, wherein the remaining features are as described above for H-L-T or the second, third, fourth, fifth, sixth, or seventh embodiment.

In a ninth embodiment, L is selected from -Het¹-X¹—, -Het¹-, -Het¹-Het²-X¹—, -Het¹-Het²-, —NR^{d}—(CH₂)ₘ—X³—NR^{c}—(CH₂ₘ-Het¹-X¹-Het²-X²—, —NR^{c}—(CH₂)ₘ-Het¹-X¹-Het²-X²—, -Het¹-X¹-Het²-X²—, O—(CH₂)ₘ—NR^{c}—X¹—(CH₂)ₘ—NR^{d}—, —X¹—NR^{c}-X²—O—(CH₂)ₘ—NR^{d}—, —X¹-Het¹-X²-Het²-(CH₂)ₘO—, O-Het¹-, O-Het¹-X¹—, —X¹(OCH₂CH₂)ₙ—NR^{c}—, —(CH₂)ₘ—NR^{c}—, —(CH₂)ₘ—, —O—, X¹NR^{c}—, —NR^{c}—(CH₂)ₘ—X¹-Het¹-X²—, —NR^{d}—(CH₂)ₘ—X³—NR^{c}—(CH₂)ₘ-Het¹-X¹-Het²-X²—, O-Het¹-X¹—(CH₂)ₘ—NR^{d}—, —X¹—NR^{c}—X²—(CH₂)ₘ—NR^{d}—, X¹-Het¹-X²—NR^{c}—X³-Het²-(OCH₂CH₂)ₙ—(CH₂)ₘ—NR^{d}—(CH₂)ₘ—, —NR^{d}—(CH₂)ₘ—X¹—NR^{c}—(CH₂CH₂O)ₙ—, —NR^{c}—(CH₂)ₘ—X¹—NR^{c}—(CH₂)ₚ—, X¹-Het¹-X²—NR^{c}—X³-Het²-(OCH₂CH₂)ₙ—NR^{d}—(CH₂)ₘ—, —NR^{c}—(CH₂)ₘ—X¹-Het¹-X²-Het²-X³—, O—X¹-Het¹-, —O(CH₂)ₘ—X¹-Het¹-X²-Het²-X³—, —O(CH₂)ₘ—X¹—NR^{c}—(CH₂)ₚ-Het¹-X²-Het²-X³—, O—(CH₂)ₘ—NR^{c}—, O—X¹-Het¹-X²—, —X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH)CH₃-Het¹-X²-Het³-X³—, —NR^{c}—(CH₂)ₘ—X¹—(CH₂)ₚ-Het¹-X²-Het²-X³—, —NR^{c}—(CH₂)ₘ—X¹—NR^{d}—(CH₂)ₚ-Het¹-X²-Het²-X³—, —NR^{c}—(CH₂)ₘ—NR^{d}—X¹-Het¹-X²—, Het¹-X¹-Het²-X²—, -Het¹-X¹-Het²-X²—O—, —O(CH₂)ₘ-Het¹-(CH₂)ₚ—O(CH₂)ₘ—NR^{c}—X²—, —O(CH₂)ₘ-Het¹-(CH₂)ₚ—O(CH₂)ₘ—NR^{c}—X²—, -Het¹-O—(CH₂)ₘ—X¹-Het²-X²—, -Het¹-O—(CH₂)ₘ—X¹—NR^{c}—(CH₂CH₂O)ₙ(CH₂)ₘ-Het²-X²—, -Het¹-X¹—NR^{c}—(CH₂)ₘ—, -Het¹-X¹-Het²-Het³-X²—, -Het¹-X¹—NR^{c}—(CH₂CH₂O)ₙ(CH₂)ₘ—, -Het¹-X¹—NR^{c}—(CH₂CH₂O)ₙ-Het²-(CH₂)ₘ—X²—, -Het¹-X¹—NR^{c}—(CH₂CH₂O)ₙ—, -Het¹-X¹—NR^{c}—(CH₂)ₘ-Het²-X²-Het³-(CH₂)ₘ—, -Het¹-X¹-Het²-(CH₂)ₘ-Het³-X²—, -Het¹-X¹-Het²-, -Het¹-X¹—NR^{c}—, -Het¹-X¹—NR^{c}—(CH₂)ₘ-Phe-X²-Het²-(CH₂)ₘ—, -Het¹-X¹-Het²-Het³-, -Het¹-X¹-Het²-(CH₂)ₘ-Het³-X²—(CH₂)ₚ—NR^{c}—(CH₂)ₘ—, -Het¹-X¹-Het²-(CH₂)ₘ-Het³-(CH₂)ₘ—O—, -Het¹-X¹-Het²-(CH₂)ₘ-Het³-(CH₂)ₚ—NR^{c}—(CH₂)ₘ—, -Het¹-X¹-Het²-(CH₂CH₂O)ₙ—, -Het¹-X¹—(CH₂)ₘ-Het²-X²—, —(CH₂CH₂O)ₒ—(CH₂)ₚ-Het¹-X¹-Het²-(CH₂CH₂O)ₙ, —(CH₂CH₂O)ₙ(CH₂)ₘ-Het¹-X¹-Het²-X², -Het¹-X¹-Phe-X²—NR^{c}—X³—, —(CH₂CH₂O)ₒ—(CH₂)ₚ-Het¹-X¹-Phe-X²—NR^{c}—(CH₂CH₂O)ₙ—, —(CH₂CH₂O)ₙ—(CH₂)ₘ—NR^{c}-Phe-X¹—, —(CH₂CH₂O)ₒ—(CH₂)ₚ—NR^{c}-Phe-(CH₂CH₂O)ₙ—, —(CH₂CH₂O)ₒ—(CH₂)ₚ—NR^{c}—(CH₂CH₂O)ₙ—(CH₂)ₘ—, —(CH₂CH₂O)ₙ—(CH₂)ₘ—NR^{c}—(CH₂CH₂O)ₘ—(CH₂)ₘ—C(O)—NR^{d}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹-Het²-X²—, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, —NR^{c}—(CH₂CH₂O)ₙ—(CH₂)ₘ-Phe-NH—X¹-Het¹-X², —NR^{c}—(CH₂CH₂O)ₙ—(CH₂)ₘ-Phe-NH—X¹-Het¹-X²—(CH₂CH₂O)ₒ, —(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹—, —(CH₂CH₂O)ₒ—(CH₂)ₚ—NR^{c}—(CH₂CH₂O)ₙ—(CH₂)ₘ-Het¹-X¹—(CH₂CH₂O)ₙ—, —(CH₂CH₂O)ₙ—(CH₂)ₘ—NR^{c}—(CH₂)ₘ—C(O)-NR^{d}-Het¹-X¹-Het²-(CH₂CH₂O)ₒ—(CH₂)ₚ, or —NR^{c}—(CH₂)ₘ—C(O)—NR^{d}—(CH₂)ₘ-Het¹-X¹-Het²-X²—, C(O)O— —X¹-Het¹-(CH₂CH₂O)ₒ—(CH₂)ₘ—NR^{c}—, -Het¹-(CH₂)ₘ-Het²-, -Het¹-X¹-Het²-(CH₂)ₚ—O—(CH₂)ₘ-, O(CH₂)ₘC(O), —OC(O)—NR^{c}—(CH₂)ₘ—NR^{d}—, —OC(O)—NR^{c}—(CH₂)ₘ—O—(CH₂)ₘ—NR^{d—}, OC(O)Het¹, —OC(O)—NR^{c}—(CH₂CH₂O)ₒ—NR^{d}—, OC(O)Het¹-Het²-, —OC(O)—NR^{c}—(CH₂)ₘC(O)-Het¹-X¹-Het²-, O—(CH₂)ₘ-Het¹-, and O—(CH₂)ₘ-Het¹-X¹-Het²;
Het¹, Het², and Het³ are each independently phenyl, a 4- to 6-membered heterocyclyl, 5- to 7-membered heteroaryl, or a 4- to 6-membered cycloalkyl, each of which are optionally substituted with (C₁-C₄)alkyl;
X¹, X², and X³, are each independently C(O) or (CH₂)ᵣ;
R^{c} and R^{d} are each independently hydrogen, (C₁-C₄)alkyl, or halo(C₁-C₄)alkyl; and
m, n, o, p, q and r are each independently integers selected from 0, 1, 2, 3, 4, 5, and 6, wherein the remaining features are as described above for H-L-T or the second, third, fourth, fifth, sixth, seventh, or eighth embodiment. Alternatively, as part of a ninth embodiment, L is selected from -Het¹-X¹-*, -Het¹-, -Het¹-Het²-X¹-*, *-Het¹-Het²-, —NR^{d}—(CH₂)ₘ—X³—NR^{c}—CH₂)ₘ-Het¹-X¹-Het²-X²-*, —NR^{c}—(CH₂)ₘ-Het¹-X¹-Het²-X²-*, -Het¹-X¹-Het²-X²-*, *O—(CH₂)ₘ—NR^{c}—X¹—(CH₂)ₘ—NR^{d}—, *-X¹—NR^{c}—X²—O—(CH₂)ₘ—NR^{d}—, *X¹-Het¹-X²-Het²-(CH₂)ₘO—, *O-Het¹-, *O-Het¹-X¹—, *-X¹(OCH₂CH₂)ₙ—NR^{c}—, *-(CH₂)ₘNR^{c}—, —(CH₂)ₘ—, —O—, *X¹NR^{c}—, —NR^{c}—(CH₂)ₘ—X¹-Het¹-X²-*, —NR^{d}—(CH₂)ₘ—X³—NR^{c}—(CH₂)ₘ-Het¹-X¹-Het²-X²-*, *O-Het¹-X¹—(CH₂)ₘ—NR^{d}—, *-X¹—NR^{c}—X²—(CH₂)ₘ—NR^{d}—, *X¹-Het¹-X²—NR^{c}—X³-Het²-(OCH₂CH₂)ₙ—(CH₂)ₘ—NR^{d}—(CH₂)ₘ—, —NR^{d}—(CH₂)ₘ—X¹—NR^{c}—(CH₂CH₂O)ₙ-*, —NR^{c}—(CH₂)ₘ—X¹—NR^{c}—(CH₂)ₚ-*, *X¹-Het¹-X²—NR^{c}—X³-Het²-(OCH₂CH₂)ₙ—NR^{d}—(CH₂)ₘ—, —NR^{c}—(CH₂)ₘ—X¹-Het¹-X²-Het²-X³-*, *O—X¹-Het¹-, —O(CH₂)ₘ—X¹-Het¹-X²-Het²-X³-*, —O(CH₂)ₘ—X¹—NR^{c}—(CH₂)ₚ-Het¹-X²-Het²-X³-*, *O—(CH₂)ₘ—NR^{c}—, *O—X¹-Het¹-X²—, *-X¹—NR^{c}—(CH₂)ₘ—Het¹-X²-Het²-X³—(CH₂)ₚ—NR^{d}—(CH₂)ₚ—, —NR^{c}—(CH₂)ₘ—X¹—(CH)CH₃-Het¹-X²-Het³-X³-*, —NR^{c}—(CH₂)ₘ—X¹—(CH₂)ₚ-Het¹-X²-Het²-X³-*, —NR^{c}—(CH₂)ₘ—X¹—NR^{d}—(CH₂)ₚ-Het¹-X²-Het²-X³-*, —NR^{c}—(CH₂)ₘ—NR^{d}—X¹-Het¹-X²-*, *Het¹-X¹-Het²-X²—, *-Het¹-X¹-Het²-X²—O—, —O(CH₂)ₘ-Het¹- (CH₂)ₚ—O(CH₂)ₘ—NR^{c}—X²-*, *-O(CH₂)ₘ-Het¹-(CH₂)ₚ—O(CH₂)ₘ—NR^{c}—X²—, *-Het¹-O—(CH₂)ₘ—X¹-Het²-X²—, *-Het¹-O—(CH₂)ₘ—X¹—NR^{c}—(CH₂CH₂O)ₙ(CH₂)ₘ-Het²-X²—, *-Het¹-X¹—NR^{c}—(CH₂)ₘ—, *-Het¹-X¹-Het²-Het³-X²—, *-Het¹-X¹—NR^{c}—(CH₂CH₂O)ₙ(CH₂)ₘ—, *-Het¹-X¹—NR^{c}—(CH₂CH₂O)ₙHet²-(CH₂)ₘ—X²—, *-Het¹-X¹—NR^{c}—(CH₂CH₂O)ₙ—, *-Het¹-X¹—NR^{c}—(CH₂)ₘ-Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³-, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ-Het³-(CH₂)ₚ—NR^{c}—(CH₂)ₘ—, *-Het¹-X¹-Het²-(CH₂CH₂O)ₙ—, *-Het¹-X¹—(CH₂)ₘ-Het²-X²—, *-(CH₂CH₂O)ₒ—(CH₂)ₚ-Het¹-X¹-Het²-(CH₂CH₂O)ₙ—, *-(CH₂CH₂O)ₙ—(CH₂)ₘ-Het¹-X¹-Het²-X², *-Het¹-X¹-Phe-X²—NR^{c}—X³—, *-(CH₂CH₂O)ₒ—(CH₂)ₚ-Het¹-X¹-Phe-X²—NR^{c}—(CH₂CH₂O)ₙ—, *-(CH₂CH₂O)ₙ—(CH₂)ₘ—NR^{c}-Phe-X¹—, *-(CH₂CH₂O)ₒ—(CH₂)ₚ—NR^{c}-Phe-(CH₂CH₂O)ₙ—, *-(CH₂CH₂O)ₒ—(CH₂)ₚ—NR^{c}—(CH₂CH₂O)ₙ—(CH₂)ₘ—, *-(CH₂C₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹-Het²-X²—, *-(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, *-NR^{c}—(CH₂CH₂O)ₙ—(CH₂)ₘ-Phe-NH—X¹-Het¹-X², *-NR^{c}—(CH₂CH₂O)ₙ—(CH₂)ₘ-Phe-NH—X¹-Het¹-X²—(CH₂CH₂O)ₒ, *-(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹—, *-(CH₂CH₂O)ₒ—(CH₂)ₚ—NR^{c}—(CH₂CH₂O)ₙ—(CH₂)ₘ-Het¹-X¹—(CH₂CH₂O)ₙ—, *-(CH₂CH₂O)ₙ—(CH₂)ₘ—NR^{c}—(CH₂)ₘ—C(O)—NR^{d}-Het¹-X l -Het²-(CH₂CH₂O)ₒ—(CH₂)ₚ, or *-NR^{c}—(CH₂)ₘ—C(O)—NR^{d}—(CH₂)ₘ-Het¹-X¹-Het²-X²—, *C(O)O—, *-X¹-Het¹-(CH₂CH₂O)ₒ—(CH₂)ₘ—NR^{c}—, -Het¹-(CH₂)ₘ-Het²-, *-Het¹-X¹-Het²-(CH₂)ₚ—O—(CH₂)ₘ-*, *O(CH₂)ₘC(O), *-OC(O)—NR^{c}—(CH₂)ₘ—NR^{d}—, *-OC(O)—NR^{c}—(CH₂)ₘ—O—(CH₂)ₘ—NR^{d—}, *OC(O)Het¹, *-OC(O)—NR^{c}—(CH₂CH₂O)ₒ—NR^{d}—, *OC(O)Het¹-Het²-, *-OC(O)—NR^{c}—(CH₂)ₘC(O)-Het¹-X¹-Het²-, *O—(CH₂)ₘ-Het¹-, and *O—(CH₂)ₘ-Het¹-X¹-Het²; and the * indicates the point of attachment to H, wherein the remaining features are as described above for H-L-T or the second, third, fourth, fifth, sixth, seventh, or eighth embodiment. Alternatively, as part of a ninth embodiment, L is selected from *-Het¹-X¹-Het²-X²—, -Het¹-X¹-Het²-X²-*, *-(CH₂)ₘNR^{c}—, —(CH₂)ₘ—, *-Het¹-X¹-Het²-, *-Het¹-Het²-, *-(CH₂CH₂O)ₙ—NR^{c}—, *-X¹-Het¹-(CH₂CH₂O)ₒ—(CH₂)ₘ—NR^{c}—, -Het¹-X¹-*, -Het¹-Het²-X¹-*, *-X¹—NR^{c}—X²—O—(CH₂)ₘ—NR^{d}—, —NR^{c}—(CH₂)ₘ-Het¹-X¹-Het²-X²-*, —NR^{d}—(CH₂)ₘ—X³—NR^{c}—(CH₂)ₘ-Het¹-X¹-Het²-X²-*, —NR^{d}—(CH₂)ₘ—X³—NR^{c}—(CH₂)ₘ-Het¹-X¹-Het²-X²-*, *O—(CH₂)ₘ—NR^{c}—X¹—(CH₂)ₘ—NR^{d—}, *-X¹(OCH₂CH₂)ₙ—NR^{c}—, —NR^{d}—(CH₂)ₘ—X¹—NR^{c}—(CH₂CH₂O)ₙ-*, *-X¹—NR^{c}—X²—(CH₂)ₘ—NR^{d—}, -Het¹-(CH₂)ₘ-Het²-*, *O—(CH₂)ₘ—NR^{c}, *O—X¹-Het¹-X²—, -Het¹-X¹-*, *O—X¹-Het¹-, *-Het¹-X¹-Phe-X²—NR^{c}—X³—, *X¹-Het¹-X²—NR^{c}—X³-Het²-(OCH₂CH₂)ₙ—NR^{d}—(CH₂)ₘ—, *X¹-Het¹-X²—NR^{c}—X³-Het²-(OCH₂CH₂)ₙ—(CH₂)ₘ—NR^{d}—(CH₂)ₘ—, *O-Het¹-, *O-Het¹-X¹—, *O-Het¹-X¹—(CH₂)ₘ—NR^{d}—, *C(O)O—, -Het¹-, —O—, *-Het¹-X¹-Het²-(CH₂)ₚ—O—(CH₂)ₘ-*, *O(CH₂)ₘC(O), *-OC(O)—NR^{c}—(CH₂)ₘ—NR^{d}—, *-OC(O)—NR^{c}—(CH₂)ₘ—O—(CH₂)ₘ—NR^{d}—, *OC(O)Het¹, *-OC(O)—NR^{c}—(CH₂CH₂O)ₒ—NR^{d}—, *OC(O)Het¹-Het²-, *-OC(O)—NR^{c}—(CH₂)ₘC(O)-Het¹-X¹-Het²-, *O—(CH₂)ₘ-Het¹-, and *O—(CH₂)ₘ-Het¹-X¹-Het², wherein the remaining features are as described above for H-L-T or the second, third, fourth, fifth, sixth, seventh, or eighth embodiment. Alternatively, as part of a ninth embodiment, L is selected from

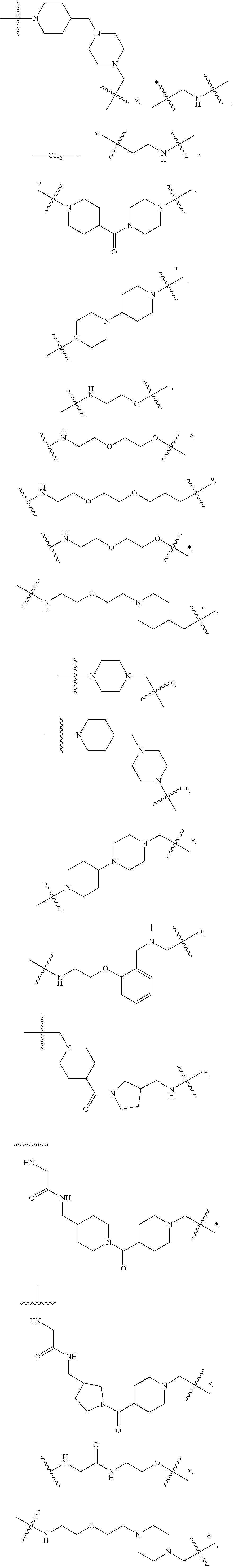

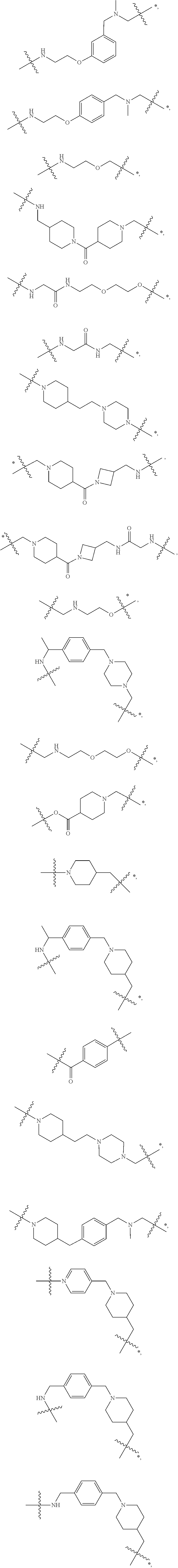

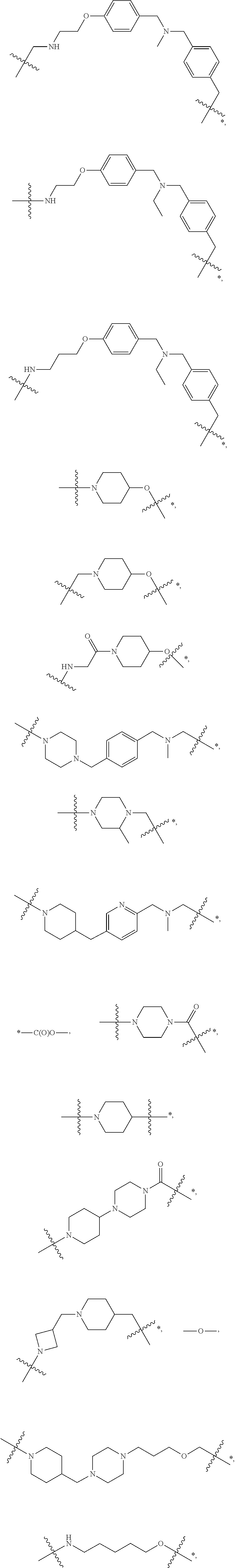

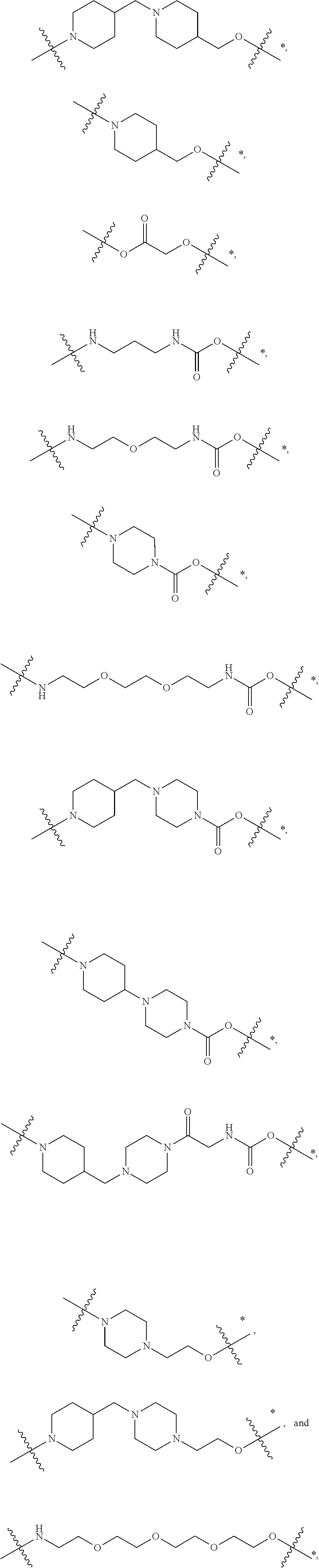

wherein the remaining features are as described above for H-L-T or the second, third, fourth, fifth, sixth, seventh, or eighth embodiment.

In a tenth embodiment, Het¹ and Het² as described in the ninth embodiment, are each independently phenyl or a 4- to 6-membered heterocyclyl. Alternatively, as part of a ninth embodiment, Het i and Het² as described in the ninth embodiment, are each independently piperidinyl, phenyl, azetidinyl, piperazinyl, or pyrrolidinyl.

In an eleventh embodiment, m, n, o, p, q and r as described in the ninth or tenth embodiment are each independently integers selected from 0, 1, 2, and 3.

In a twelfth embodiment, the target protein binder is a binder of BET, wherein the remaining features are as described above for H-L-T or the second, third, fourth, fifth, sixth, seventh, eighth, ninth, tenth, or eleventh embodiment. Alternatively, as part of a twelfth embodiment, the target protein binder is of the Formula:

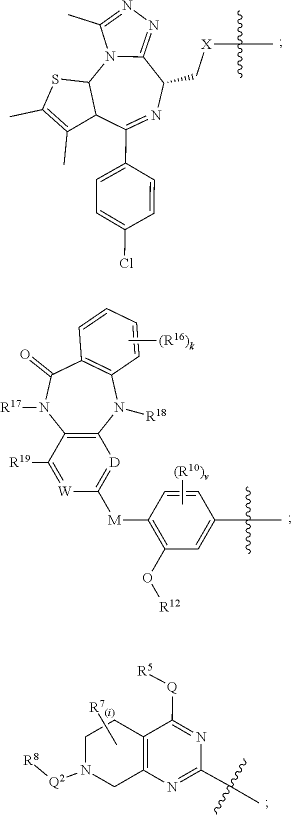

wherein
X is C(O) or (C₁-C₄)alkylene; Q¹ is a nitrogen containing heteroaryl or heterocyclyl ring, each of which are optionally substituted with 1 to 3 groups selected from R⁶;
R⁵ is —C(O)Y or —S(O)₂Y;
Y is a (C₁-C₆)alkyl, halo(C₁-C₆)alkyl, (C₂-C₆)alkenyl, halo(C₂-C₆)alkenyl, NH₂, —NH(C₁-C₆)alkyl, —N[(C₁-C₆)alkyl]₂, NHNH₂, or NHOH, wherein said (C₂-C₆)alkenyl, alone or as recited in halo(C₂-C₆)alkenyl, is optionally substituted with (C₁-C₆)alkyl, halo(C₁-C₆)alkyl, heteroalkyl, hydroxy(C₁-C₆)alkyl, —C(O)NH₂, —C(O)NH(C₁-C₆)alkyl, or —C(O)N[(C₁-C₆)alicyl]₂;
R⁶ is (C₁-C₆)alkyl, halo(C₁-C₆)alkyl, (C₁-C₆)alkoxy, halo(C₁-C₆)alkoxy, hydroxy(C₁-C₆)alkyl, cyano(C₁-C₆)alkyl, oxo, cyano, heteroalkyl, —C(O)OH, —C(O)O(C₁-C₆)alkyl, —C(O)NH₂, —C(O)NH(C₁-C₆)alkyl, or —C(O)N[(C₁-C₆)alkyl]₂, wherein said (C₁-C₆)alkyl is optionally substituted with heteroaryl;
R⁷ is halo, hydroxyl, (C₁-C₆)alkyl, halo(C₁-C₆)alkyl, (C₁-C₆)alkoxy, halo(C₁-C₆)alkoxy, cylcoalkyl, heteroalkyl, hydroxy(C₁-C₆)alkyl, or S(C₁-C₆)alkyl;
j is 1 or 2;
Q² is a bond, —C(O)—, or (C₁-C₃)alkylene;
R⁸ is cycloalkyl, heterocyclyl, aryl, or heteroaryl, each of which are optionally substituted with 1 to 3 groups selected from R⁹;
R⁹ is halo, (C₁-C₆)alkyl, (C₂-C₆)alkenyl, halo(C₂-C₆)alkenyl, (C₂-C₆)alkynyl, oxo, cyano, —(C₁-C₆)alkylOR^{c}, —(C₁-C₆)alkylN(R^{d})₂, —(C₁-C₆)alkylC(O)OR^{d}, OH, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, or CN, wherein each aryl, cycloalkyl, heterocyclyl, and heteoaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e}; and
R^{e} is selected from halo, oxo, CN, NO₂, —N(R^{d})₂, —OR^{d}, —C(O)OR^{d}, (C₁-C₆)alkyl, —(C₁-C₆)alkylOR^{c}, halo(C₁-C₆)alkyl, (C₁-C₆)alkoxy, halo(C₁-C₆)alkoxy, —(C₁-C₆)alkylC(O)OR^{d}, —(C₁-C₆)alkylC(O)N(R^{d})₂, (C₂-C₆)alkenyl, halo(C₂-C₆)alkenyl, (C₂-C₆)alkynyl, —(C₁-C₆)alkylSR^{d}, —(C₁-C₆)alkylOR^{c}, —(C₁-C₆)alkylN(R^{d})₂, —C(O)N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, aryl, heteroaryl, cycloalkyl, and heterocycloalkyl.
R¹⁶, R¹⁹, and R¹⁰ are each independently selected from halo, (C₁-C₆)alkyl, (C₂-C₆)alkenyl, halo(C₂-C₆)alkenyl, (C₂-C₆)alkynyl, —(C₁-C₆)alkylOR^{c}, —(C₁-C₆)alkylN(R^{d})₂, —(C₁-C₆)alkylC(O)OR^{d}, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, and CN, wherein each aryl, cycloalkyl, heterocyclyl, and heteoaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e};
W and D are each independently N or CR²⁰;
M is O, S, or NR¹¹;
R¹¹, R¹⁷, R¹⁸, and R²⁰, are each independently selected from hydrogen, (C₁-C₆)alkyl, and S(O)₂(C₁-C₆)alkyl;
R¹² is hydrogen, (C₁-C₆)alkyl, halo(C₁-C₆)alkyl, —(C₁-C₆)alkylOR^{c}, S(O)₂(C₁-C₆)alkyl, aryl, heteroaryl, cycloalkyl, heterocycloalkyl, C(O)(C₁-C₆)alkyl, or —(C₁-C₆)alkylaryl, wherein each aryl, cycloalkyl, heterocyclyl, and heteoaryl alone and in connection with —(C₁-C₆)alkylaryl are optionally substituted with 1 to 3 groups selected from R^{e};
and
k and v are each independently 0, 1, 2, or 3, wherein the remaining features are as described above for H-L-T or the second, third, fourth, fifth, sixth, seventh, eighth, ninth, tenth, or eleventh embodiment.

In a thirteenth embodiment, the target protein binder is of the Formula:

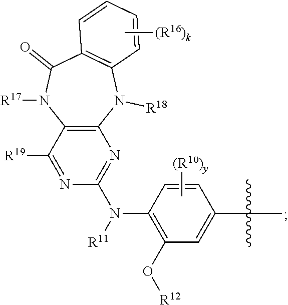

or a pharmaceutically acceptable salt thereof, wherein the remaining features are as described above for H-L-T or the second, third, fourth, fifth, sixth, seventh, eighth, ninth, tenth, eleventh, or twelfth embodiment. Alternatively, as part of a thirteenth embodiment, the target protein binder is of the Formula:

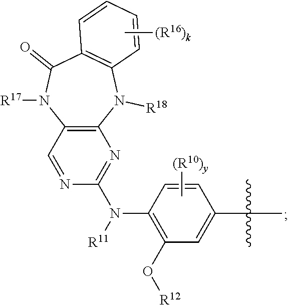

or a pharmaceutically acceptable salt thereof, wherein the remaining features are as described above for H-L-T or the second, third, fourth, fifth, sixth, seventh, eighth, ninth, tenth, eleventh, or twelfth embodiment.

In a fourteenth embodiment, k is 0, wherein the remaining features are as described above for H-L-T or the second, third, fourth, fifth, sixth, seventh, eighth, ninth, tenth, eleventh, twelfth, or thirteenth embodiment.

In a fifteenth embodiment, v is 0, wherein the remaining features are as described above for H-L-T or the second, third, fourth, fifth, sixth, seventh, eighth, ninth, tenth, eleventh, twelfth, thirteenth, or fourteenth embodiment.

In a sixteenth embodiment, R¹¹ is hydrogen, wherein the remaining features are as described above for H-L-T or the second, third, fourth, fifth, sixth, seventh, eighth, ninth, tenth, eleventh, twelfth, thirteenth, fourteenth, or fifteenth embodiment.

In a seventeenth embodiment, R¹⁷ is (C₁-C₆)alkyl, wherein the remaining features are as described above for H-L-T or the second, third, fourth, fifth, sixth, seventh, eighth, ninth, tenth, eleventh, twelfth, thirteenth, fourteenth, fifteenth, or sixteenth embodiment. Alternatively, as part of a seventeenth embodiment, R¹⁷ is methyl, wherein the remaining features are as described above for H-L-T or the second, third, fourth, fifth, sixth, seventh, eighth, ninth, tenth, eleventh, twelfth, thirteenth, fourteenth, fifteenth, or sixteenth embodiment.

In an eighteenth embodiment, R¹² is (C₁-C₆)alkyl, wherein the remaining features are as described above for H-L-T or the second, third, fourth, fifth, sixth, seventh, eighth, ninth, tenth, eleventh, twelfth, thirteenth, fourteenth, fifteenth, sixteenth, or seventeenth embodiment. Alternatively, as part of an eighteenth embodiment, R¹² is ethyl, wherein the remaining features are as described above for H-L-T or the second, third, fourth, fifth, sixth, seventh, eighth, ninth, tenth, eleventh, twelfth, thirteenth, fourteenth, fifteenth, sixteenth, or seventeenth embodiment.

In a nineteenth embodiment, R¹⁸ is (C₁-C₃)alkyl or S(O)₂(C₁-C₃)alkyl, wherein the remaining features are as described above for H-L-T or the second, third, fourth, fifth, sixth, seventh, eighth, ninth, tenth, eleventh, twelfth, thirteenth, fourteenth, fifteenth, sixteenth, seventeenth, or eighteenth embodiment. Alternatively, as part of a nineteenth embodiment, R¹⁸ is S(O)₂Me, wherein the remaining features are as described above for H-L-T or the second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment, the target protein binder is of the Formula:

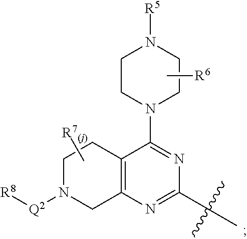

or a pharmaceutically acceptable salt thereof, wherein the remaining features are as described above for H-L-T or the second, third, fourth, fifth, sixth, seventh, eighth, ninth, tenth, eleventh, or twelfth embodiment.

In a twenty-first embodiment, R⁵ is —C(O)Y, wherein the remaining features are as described above for H-L-T or the second, third, fourth, fifth, sixth, seventh, eighth, ninth, tenth, eleventh, twelfth, or twentieth embodiment.

In a twenty-second embodiment, Y is (C₁-C₆)alkyl, halo(C₁-C₆)alkyl, (C₂-C₆)alkenyl, halo(C₂-C₆)alkenyl, or NH₂, wherein the remaining features are as described above for H-L-T or the second, third, fourth, fifth, sixth, seventh, eighth, ninth, tenth, eleventh, twelfth, twentieth, or twenty first embodiment. Alternatively, as part of a twenty-second embodiment, Y is C(O)CH₃, C(O)CHCH₂, C(O)CH₂CH₃, C(O)CF₃, C(O)CFCH₂, C(O)CCH₃, or C(O)NH₂, wherein the remaining features are as described above for H-L-T or the second, third, fourth, fifth, sixth, seventh, eighth, ninth, tenth, eleventh, twelfth, twentieth, or twenty-first embodiment. Alternatively, as part of a twenty-second embodiment, Y is C(O)CHCH₂, wherein the remaining features are as described above for H-L-T or the second, third, fourth, fifth, sixth, seventh, eighth, ninth, tenth, eleventh, twelfth, twentieth, or twenty-first embodiment.

In a twenty-third embodiment, R⁶ is cyano(C₁-C₆)alkyl, wherein the remaining features are as described above for H-L-T or the second, third, fourth, fifth, sixth, seventh, eighth, ninth, tenth, eleventh, twelfth, twentieth, twenty-first, or twenty-second embodiment. Alternatively, as part of a twenty-third embodiment, R⁶ is CH₂CN, wherein the remaining features are as described above for H-L-T or the second, third, fourth, fifth, sixth, seventh, eighth, ninth, tenth, eleventh, twelfth, twentieth, twenty-first, or twenty-second embodiment.

In a twenty-fourth embodiment, j is 0, wherein the remaining features are as described above for H-L-T or the second, third, fourth, fifth, sixth, seventh, eighth, ninth, tenth, eleventh, twelfth, twentieth, twenty-first, twenty-second, or twenty-third embodiment.

In a twenty-fifth embodiment, Q² is a bond, wherein the remaining features are as described above for H-L-T or the second, third, fourth, fifth, sixth, seventh, eighth, ninth, tenth, eleventh, twelfth, twentieth, twenty-first, twenty-second, twenty-third, or twenty-fourth embodiment.

In a twenty-sixth embodiment, R⁸ is aryl optionally substituted with 1 to 3 groups selected from R⁹, wherein the remaining features are as described above for H-L-T or the second, third, fourth, fifth, sixth, seventh, eighth, ninth, tenth, eleventh, twelfth, twentieth, twenty-first, twenty-second, twenty-third, twenty-fourth, or twenty-fifth embodiment. Alternatively, as part of a twenty-sixth embodiment, R⁸ is naphthyl optionally substituted with 1 to 3 groups selected from R⁹, wherein the remaining features are as described above for H-L-T or the second, third, fourth, fifth, sixth, seventh, eighth, ninth, tenth, eleventh, twelfth, twentieth, twenty-first, twenty-second, twenty-third, twenty-fourth, or twenty-fifth embodiment.

In a twenty-seventh embodiment, R⁹ is selected from halo, (C₁-C₆)alkyl, and OH, wherein the remaining features are as described above for H-L-T or the second, third, fourth, fifth, sixth, seventh, eighth, ninth, tenth, eleventh, twelfth, twentieth, twenty-first, twenty-second, twenty-third, twenty-fourth, twenty-fifth, or twenty-sixth embodiment. Alternatively, as part of a twenty-seventh embodiment, R⁹ is selected from chloro and OH, wherein the remaining features are as described above for H-L-T or the second, third, fourth, fifth, sixth, seventh, eighth, ninth, tenth, eleventh, twelfth, twentieth, twenty-first, twenty-second, twenty-third, twenty-fourth, twenty-fifth, or twenty-sixth embodiment.

Also provided herein are compounds of the Formula H-L, wherein H and L are as defined as described above for H-L-T or the second, third, fourth, fifth, sixth, seventh, eighth, ninth, tenth, or eleventh embodiment.

Also provided herein are compounds of the Formula H-L-P, wherein H and L are as defined as described above for H-L-T or the second, third, fourth, fifth, sixth, seventh, eighth, ninth, tenth, or eleventh embodiment; and P is a protecting group.

Specific compounds are exemplified below and are included as part of the invention. Free base and salt forms of these compounds are also included.

### 4. Uses, Formulation and Administration

Compounds and compositions described herein are generally useful as anticancer therapies. In one aspect, the disclosed compounds and compositions behave as chaperone-mediated protein degraders (CHAMPs) in which one portion of the compounds is responsible for binding a target protein or proteins and the other portion is responsible for binding to HSP90 or other chaperone proteins or protein components of chaperone complexes (e.g., members of the HSP70 family). Their mechanisms of action include, but are not limited to, degrading a target protein or proteins and thereby impeding processes that may result in inhibition of cancer cell growth and/or induction of cancer cell death or other functions dependent on the target protein(s). In one aspect, the disclosed compounds effectuate the degradation of the target protein(s).

In one aspect, the disclosed compounds and compositions include chaperone or chaperone complex binders that have a range of different binding affinities. In different embodiments, it is desirable to use a high-affinity binder, a moderate-affinity binder or a low-affinity binder. Since a HSP90-binding moiety that interacts with the N-terminal ATP-binding pocket of HSP90 may inhibit HSP90 activity and induce the degradation of HSP90 client proteins (Schopf et al., Nat Rev Mol Cell Biol, 2017, 18:345-360), some CHAMP molecules may not only induce the degradation of the desired target protein or proteins (which may or may not be HSP90 client proteins), but also simultaneously induce the degradation of HSP90 client proteins. EGFR and ERBB2 (HER2) are two such HSP90 client proteins (Xu et al., J Biol Chem, 2001, 276:3702-3708). Such combinations of degradation activities may increase the biological activity of CHAMP molecules over that of other TPD technologies directed towards the same target(s) and may evade mechanisms of resistance to other degraders and inhibitors of the target protein(s) that are mediated by such HSP90 client proteins.

In one aspect, the disclosed compounds and compositions behave as tumor-targeted CHAMPs in which one portion of the compounds is responsible for binding KRAS(G12C) and the other portion is responsible for binding to HSP90 or other chaperone proteins or protein components of chaperone complexes (e.g., members of the HSP70 family). In one aspect, the disclosed compounds and compositions have prolonged pharmacokinetic exposures in cancer cells and tumors relative to normal cells, tissues and organs (Kamal et al., Nature, 2003, 425:407-410; Vilenchik et al., Chem Biol, 2004, 11:787-797). In one aspect, the disclosed compounds have increased therapeutic indexes relative to other degraders and inhibitors of the target protein(s).

Thus, provided herein are methods of treating conditions which are responsive to the degradation of the target protein or proteins comprising administering to a subject in need thereof, a therapeutically effective amount of one or more compounds or compositions described herein. Also provided is the use of one or more compounds or compositions described herein in the manufacture of a medicament for treating conditions which are responsive to the degradation of degradation of the target protein or proteins. Further provided is the use of a compound or composition described herein for treating conditions which are responsive to the degradation of a target protein or proteins.

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Example 1: Synthesis of H-L and Its Application to Synthesize H-L-T

A representative synthesis scheme for compound 147 is shown in below. Specific synthesis routes of intermediates are also shown.

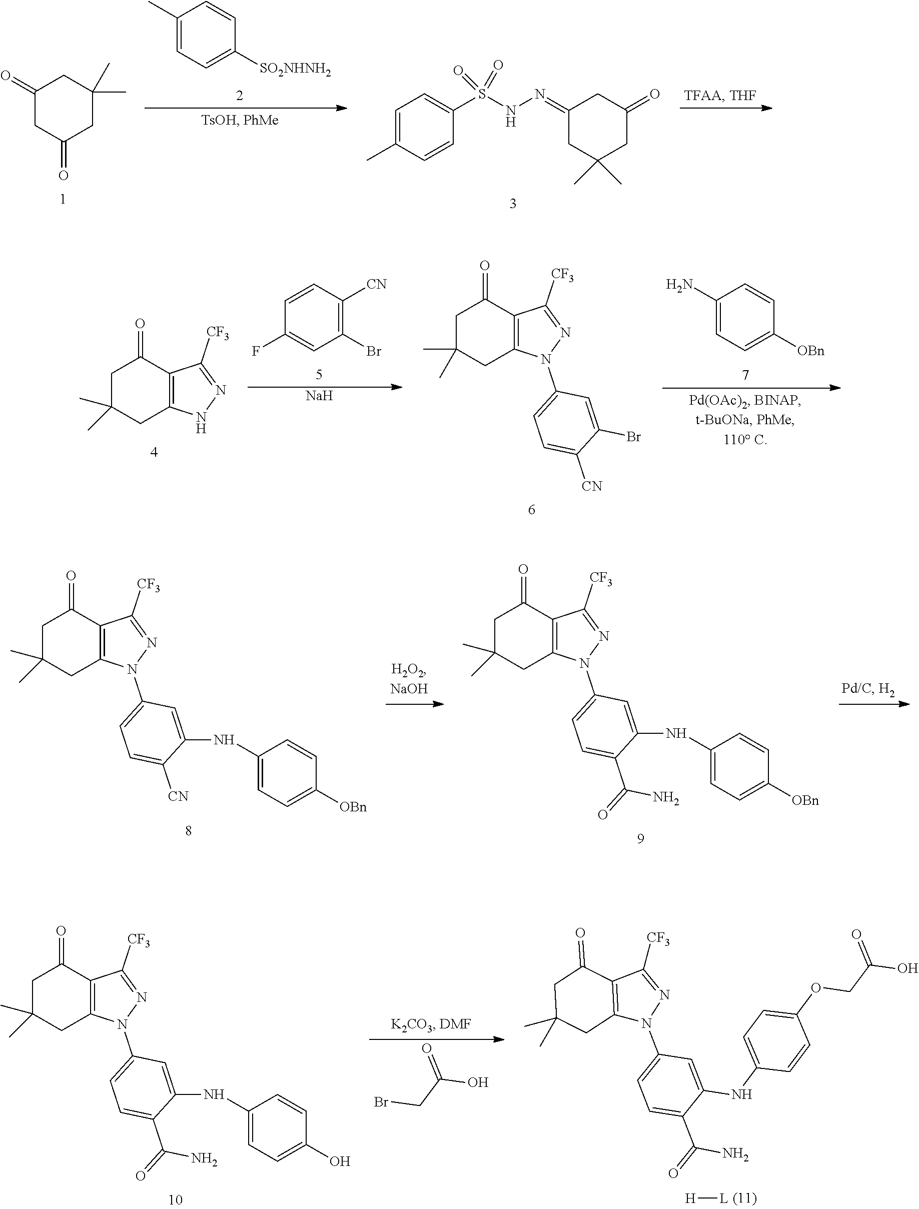

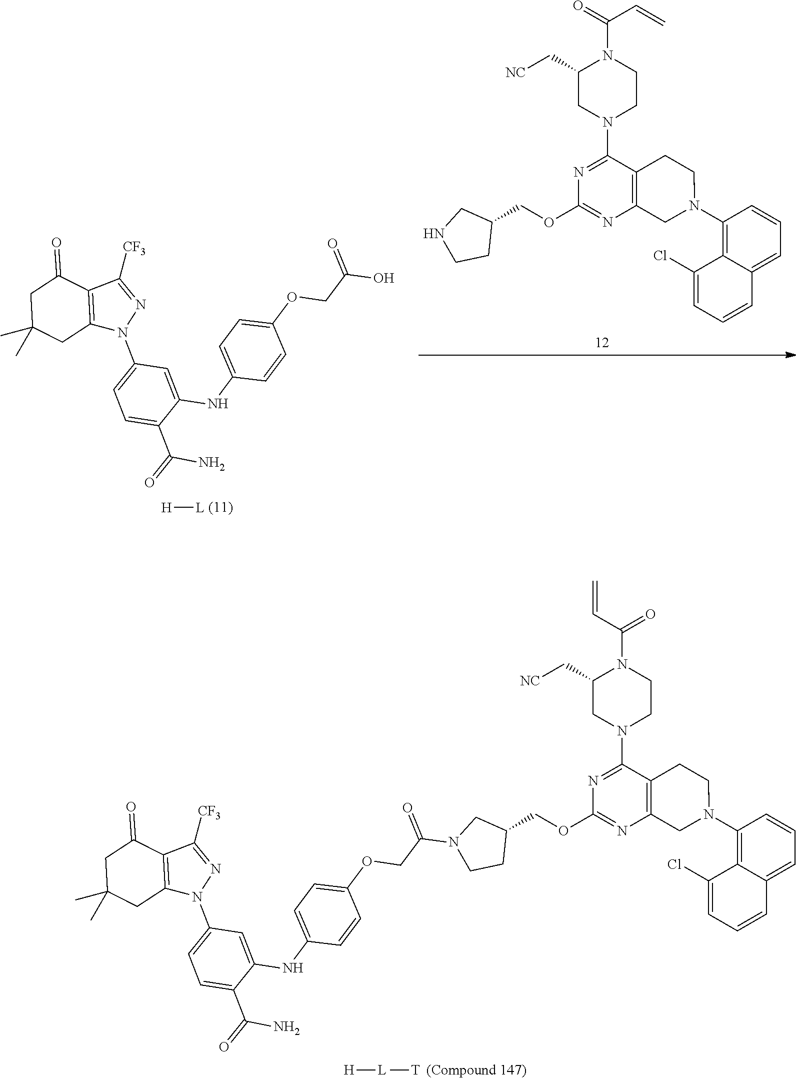

### 5 Intermediate 3

### (E)-N′-(3,3-dimethyl-5-oxocyclohexylidene)-4-methylbenzenesulfonohydrazide

A mixture of 1 (200 g, 1426.72 mmol), 2 (265.71 g, 1426.7167 mmol) and p-Toluene sulfonic acid (24.54 g, 142.67 mmol) in toluene (8 L) was heated to 120° C. After 1 h, the mixture was cooled and followed by the addition of toluene (1.2 L). The mixture was then reflux for 1 h. The reaction was cooled to ambient temperature. The precipitated solids was collected by filtration, washed three times with ether and dried under vacuum to give intermediate 3 (360 g, 1167.30 mmol, 81.82%). LCMS: m/z 309 [M+H]⁺.

### Intermediate 4

### 6,6-dimethyl-3-(trifluoromethyl)-1,5,6,7-tetrahydro-4H-indazol-4-one

To a suspension of 3 (360 g, 1167.30 mmol) and TEA (486.67 mL, 3501.33 mmol) in THF (3 L) was added trifluoroacetyl 2,2,2-trifluoroacetate (243.51 mL, 1750.67 mmol) at 0° C. The resulting reaction was heated to 55° C. for 3 h, the reaction mixture was cooled to ambient temperature. To the mixture was added Methanol (1.4 L) and 1 N NaOH (1.4 L). After stirring for 3 h, the reaction mixture was diluted with saturated ammonium chloride (3 L), extracted with ethyl acetate three times, the combine organic layers was washed with brine, dried over sodium sulfate, and concentrated in vacuum. The residue was purified by column chromatography to give the intermediate 4 (160 g, 689.05 mmol, 59.04%). LCMS: m/z 233 [M+H]⁺.

### Intermediate 6

### 2-bromo-4-(6,6-dimethyl-4-oxo-3-(trifluoromethyl)-4,5,6,7-tetrahydro-1H-indazol-1-y1)benzonitrile

NaH (15.50 g, 645.98 mmol) was added to a solution of 4 (150 g, 645.98 mmol) in DMSO (2 L) at room temperature. After 15 min, 2-bromo-4-fluorobenzonitrile (129.20 g, 645.98 mmol) was added as solid. The reaction mixture was heated at 45° C. overnight. The mixture was cooled to room temperature and quenched with saturated aqueous NH₄Cl. The mixture was diluted with water and extracted with EtOAc. The combined organic layers were washed with brine, dried over anhydrous sodium sulfate and concentrated in vacuum. The residue was purified by column chromatography to give intermediate 6 (180 g, 436.67 mmol, 67.59%). LCMS: m/z 412 [M+H]⁺.

### Intermediate 8

### 2-((4-(benzyloxy)phenyl)amino)-4-(6,6-dimethyl-4-oxo-3-(trifluoromethyl)-4,5,6,7-tetrahydro-1H-indazol-1-yl)benzonitrile

To a solution of 6 (50 g, 121.30 mmol) in toluene (500 mL) was added 7 (24.17 g, 121.30 mmol) and Cs₂CO₃ (79.04 g, 242.59 mmol). Then BINAP (15.10 g, 24.26 mmol) and Pd(OAc)₂ (2.74 g, 12.13 mmol) was added successively under nitrogen protection. The mixture reaction was heated to 120° C. for 3 h. After which in was filtered, the filtrate was concentrated in vacuum, the residue was purified by silica gel chromatography to give the intermediate 8 (40 g, 75.39 mmol, 62.16%). LCMS: m/z 531 [M+H]⁺.

### Intermediate 9

### 2-((4-(benzyloxy)phenyl)amino)-4-(6,6-dimethyl-4-oxo-3-(trifluoromethyl)-4,5,6,7-tetrahydro-1H-indazol-1-yl)benzamide

To a solution of 8 (40 g, 75.39 mmol) in EtOH (400 mL) and DMSO (100 mL) was added 1 N NaOH (226.18 mL, 226.18 mmol) and H₂O₂ (25.63 g, 226.18 mmol) dropwise successively at 0° C. Then the mixture was stirred at RT for 2 h before diluting with water, it was extracted with EtOAc, washed with brine, dried over sodium sulfate. The organic layer was concentrated in vacuum, the residue was purified by silica gel column to give the intermediate 9 (35 g, 63.80 mmol, 84.63%). LCMS: m/z 549 [M+H]⁺.

### Intermediate 10

### 4-(6,6-dimethyl-4-oxo-3-(trifluoromethyl)-4,5,6,7-tetrahydro-1H-indazol-1-yl)-2-((4-hydroxyphenyl)amino)benzamide

To a solution of 9 (35 g, 63.80 mmol) in MeOH (400 mL) was added Pd/C 10% (6.7 g, 6.38 mmol), the mixture was stirred at RT overnight with H₂ existence. After which it was filtered, washed with EA followed by DCM, the filler was concentrated in vacuum to give intermediate 10 (26 g, 56.71 mmol, 88.89%) as a solid. LCMS: m/z 459 [M+H]⁺.

### H-L (11)

### 2-(4-((2-carbamoyl-5-(6,6-dimethyl-4-oxo-3-(trifluoromethyl)-4,5,6,7-tetrahydro-1H-indazol-1-yl)phenyl)amino)phenoxy)acetic acid

To a solution of 2-bromoacetic acid (0.6 g, 4.59 mmol) in DMF (20 mL) was added 10 (1.89 g, 4.13 mmol) and K₂CO₃ (1.90 g, 13.76 mmol), the mixture was stirred at 90° C. overnight. Then water was added, it was extracted with EA, washed with saturated brine, dried over sodium sulfate, concentrated in vacuum, the residue was purified by silica gel column to give H-L (11, 1.6 g, 2.48 mmol, 54.01%) as a solid. LCMS: 518 [M+H]⁺.

### H-L-T (Compound 147)

### 2-((4-(2-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyppyrrolidin-1-yl)-2-oxoethoxy)phenyl)amino)-4-(6,6-dimethyl-4-oxo-3-(trifluoromethyl)-4,5,6,7-tetrahydro-1H-indazol-1-yl)benzamide

To a solution of H-L (11, 340 mg, 0.7 mmol), HATU (290 mg, 0.77 mmol) and DIEA (450 mg, 3.48 mmol) in DMF (8 mL) was added intermediate 12 (350 mg, 0.7 mmol). The resulting mixture was stirred at room temperature for 2 hours. The mixture was purified by prep-HPLC to give H-L-T (compound 147) (230 mg) as yellow solid. ¹H NMR (DMSO-d₆, 400 MHz): δ 10.09 (s, 1H), 8.19 (s, 1H), 7.92-7.88 (m, 2H), 7.74 (d, J=8.0 Hz, 1H), 7.58-7.53 (m, 3H), 7.50 (t, J=7.2 Hz, 1H), 7.44-7.30 (m, 1H), 7.20 (brs, 2H), 7.03 (s, 1H), 6.95-6.82 (m, 4H), 6.18 (d, J=16.4 Hz, 1H), 5.77 (d, J=10.8 Hz, 1H), 4.96-4.75 (m, 1H), 4.71 (s, 2H), 4.41-4.14 (m, 4H), 4.03-3.48 (m, 9H), 3.24-3.15 (m, 5H), 3.09 (s, 3H), 3.02-2.54 (m, 2H), 2.40 (d, J=5.6 Hz, 2H), 2.12-1.61 (m, 3H), 1.02 (s, 6H). LC-MS: m/z 1070.4 [M+H]⁺.

### Example 2: Synthesis of H-L and Its Application to Synthesize H-L-T

A representative synthesis scheme for compound 10B is shown in below. Specific synthesis routes of intermediates are also shown.

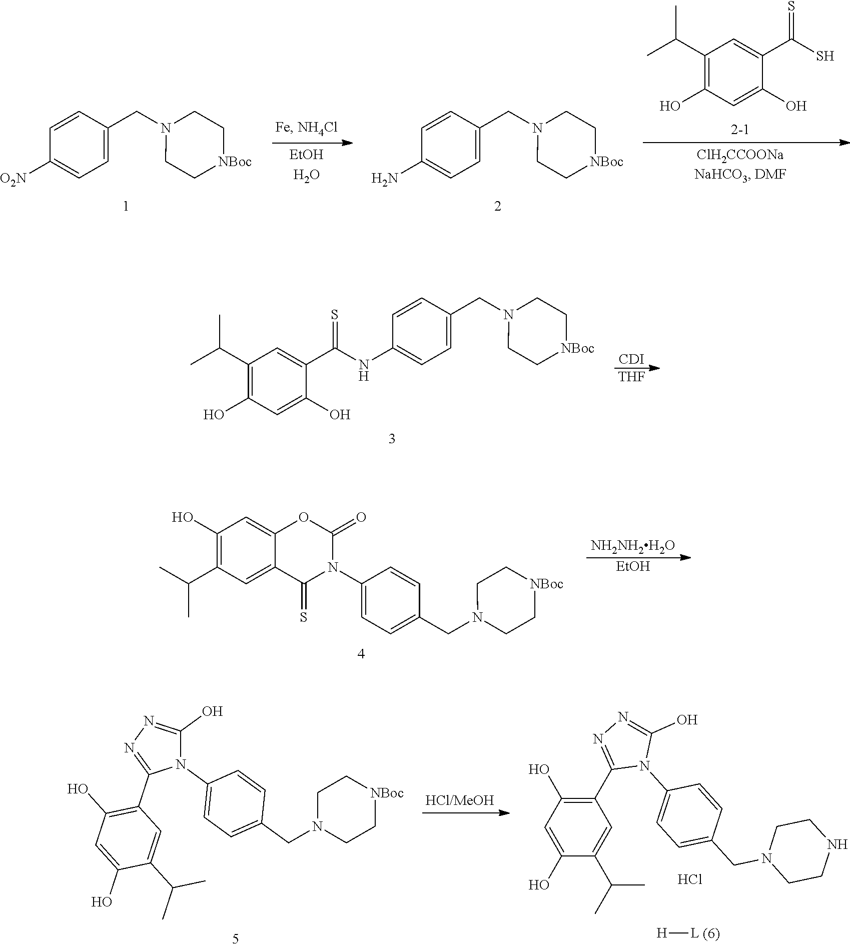

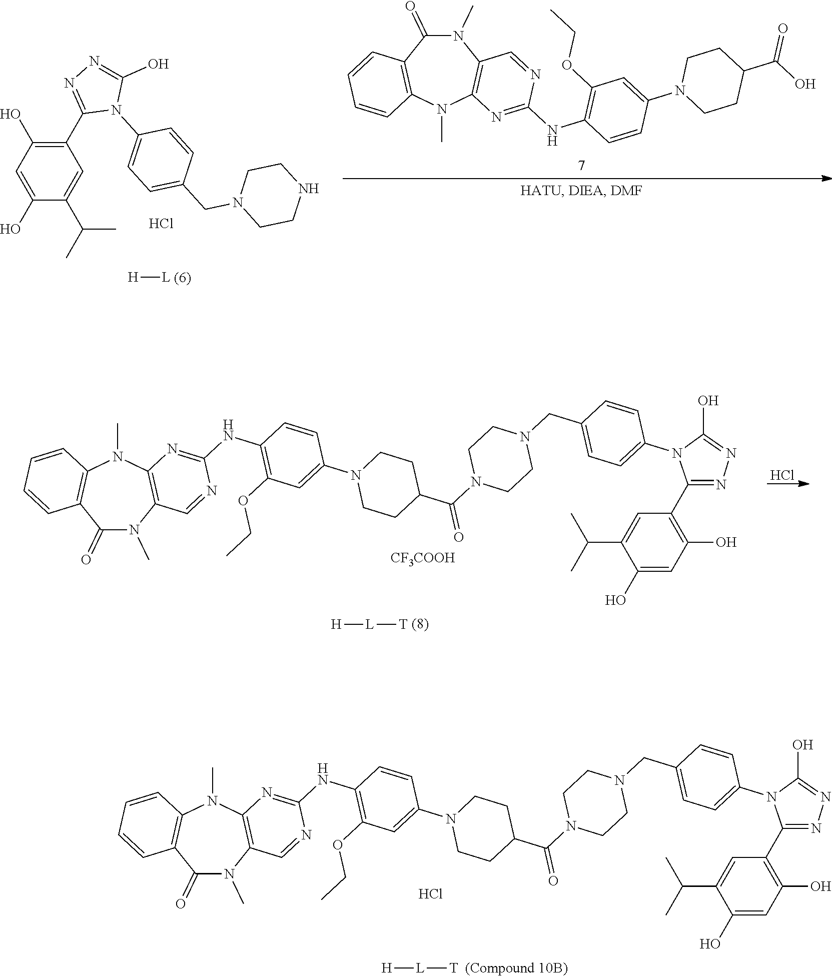

### Intermediate 2

### tert-butyl 4-(4-aminobenzyl)piperazine-1-carboxylate

To a solution of compound 1 (2.0 g, 15.6 mmol) in EtOH (15 mL) and H₂ O (5 mL) was added Fe power (1.72 g, 77.9 mmol) and NH₄Cl (3.4 g, 105.5 mmol). The resulting mixture was heated to 80° C. for 2 hours. The reaction solution was cooled to room temperature and filtered. The filtrate was poured into aq. NaHCO 3 solution, extracted with EtOAc (20 mL*3). The combine organic layers were washed with brine, dried over Na₂SO₄ and concentrated to give intermediate 2 (1.81 g, yield 100%) as a white solid.

### Intermediate 3:

### tert-butyl 4-(4-(2,4-dihydroxy-5-isopropylphenylthioamido)benzyl)piperazine-1-carboxylate

The solution of compound 2-1 (1.45 g, 6.30 mmol), ClCH₂COONa (1.09 g, 9.53 mmol) and NaHCO₃ (1.60 g, 19.1 mmol) in DMF (10 mL) was stirred for at 30° C. 3 hours. Compound 2 (1.85 g, 6.3 mmol) was added to the mixture. After the resulting mixture was heated at 80° C. for 4 hours, the reaction mixture was poured into ice-water and extracted with EtOAc (15 mL*3). The combine organic layers was washed with brine, dried over Na 2 SO 4 and filtered. The filtrate was concentrated and purified by SGC eluted with DCM:MeOH=20:1 to give intermediate 3 (2.1 g, yield 70%) as a yellow oil.

### Intermediate 4

### tert-butyl 4-(4-(7-hydroxy-6-isopropyl-2-oxo-4-thioxo-2H-benzo[e][1,3]oxazin-3(4H)-yl)benzyl)piperazine-1-carboxylate

The solution of intermediate 3 (2.1 g 4.3 mmol) and CDI (1.40 g, 8.6 mmol) in THF (15 mL) was stirred at room temperature for 4 hours. The reaction solution was poured into brine (25 mL) and extracted with EtOAc (25 mL*2). The combine organic layers was washed with brine, dried over Na₂ SO₄ and concentrated to give intermediate 4 (2.7 g, crude) which was used for further reaction without purification.

### Intermediate 5

### tert-butyl 4-(4-(3-(2,4-dihydroxy-5-isopropylphenyl)-5-hydroxy-4H-1,2,4-triazol-4-yl)benzyl)piperazine-1-carboxylate

To a solution of intermediate 4 (2.7 g, crude) in EtOH (6 mL) was added NH₂NH₂H₂O (253 mg, 7.9 mmol). The resulting mixture was stirred at room temperature overnight. The precipitated solid was filtered to give intermediate 5 (1.3 g, yield 48.5%) as a white solid.

### H-L (6)

### 4-(5-hydroxy-4-(4-(piperazin-1-ylmethyl)phenyl)-4H-1,2,4-triazol-3-yl)-6-isopropylbenzene-1,3-diol, hydrochloride

The solution of intermediate 5 (1.3 g, 2.50 mmol) in HCl/MeOH (3 N, 15 mL) was stirred at room temperature for 16 hours. The reaction solution was concentrated to give H-L (6, 1.02 g, yield 98%) as a white solid.

### H-L-T (Compound 10B)

### 2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)amino)-5,11-dimethyl-hydrochloride

To a solution of H-L (6, 340 mg, 0.7 mmol), HATU (290 mg, 0.77 mmol) and DIEA (450 mg, 3.48 mmol) in DMF (8 mL) was added intermediate 7 (350 mg, 0.7 mmol). The resulting mixture was stirred at room temperature for 2 hours. The mixture was purified by prep-HPLC to give H-L-T (8, TFA salt) as a white solid. It was added to NaHCO₃ solution and EtOAc was added then extracted. Organic phase was dried and concentrated. H₂O (10 mL) and CH₃CN (1 mL) were added to the residue followed by 3N HCl (0.17 mL). It was lyophilized to give H-L-T (compound 10B) (230 mg) as yellow solid.
¹H NMR (400 MHz, DMSO-d₆): δ 13.02 (s, 1H), 11.92 (m, 2H), 9.94-9.24 (m, 1H), 8.49 (d, J=28.4 Hz, 2H), 8.26 (d, J=8.4 Hz, 1H), 7.72-7.45 (m, 6H), 7.30-7.19 (m, 4H), 6.91 (s, 1H), 6.36 (s, 1H), 4.49-4.16 (m, 6H), 3.76-3.53 (m, 5H), 3.41 (s, 3H), 3.37 (s, 3H), 3.29 (d, J=8.4 Hz, 2H), 3.23-3.12 (m, 2H), 3.10-2.89 (m, 3H), 2.29 (s, 2H), 1.94 (s, 2H), 1.40 (t, J=7.2 Hz, 3H), 1.03 (d, J=6.8 Hz, 6H). LCMS (ESI): RT=1.080 min, m/z found 894.3 [M-HCl+H]⁺.

### Example 3: Synthesis of H-L and Its Application to Synthesize H-L-T

A representative synthesis scheme for compound 168 is shown in below. Specific synthesis routes of intermediates are also shown.

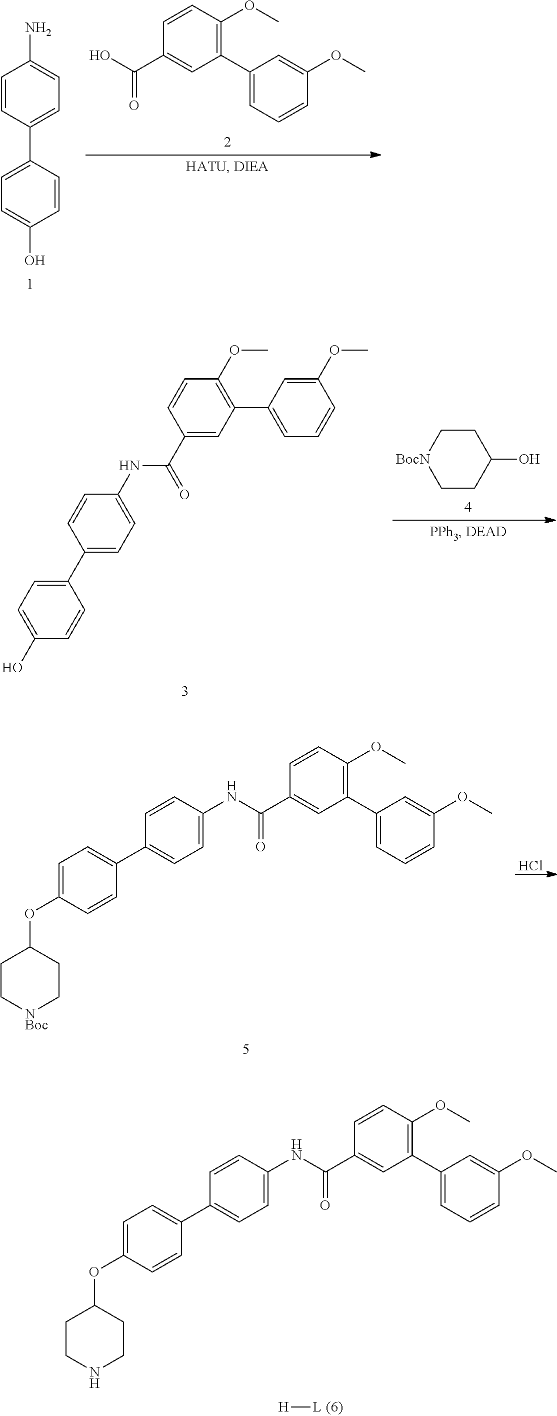

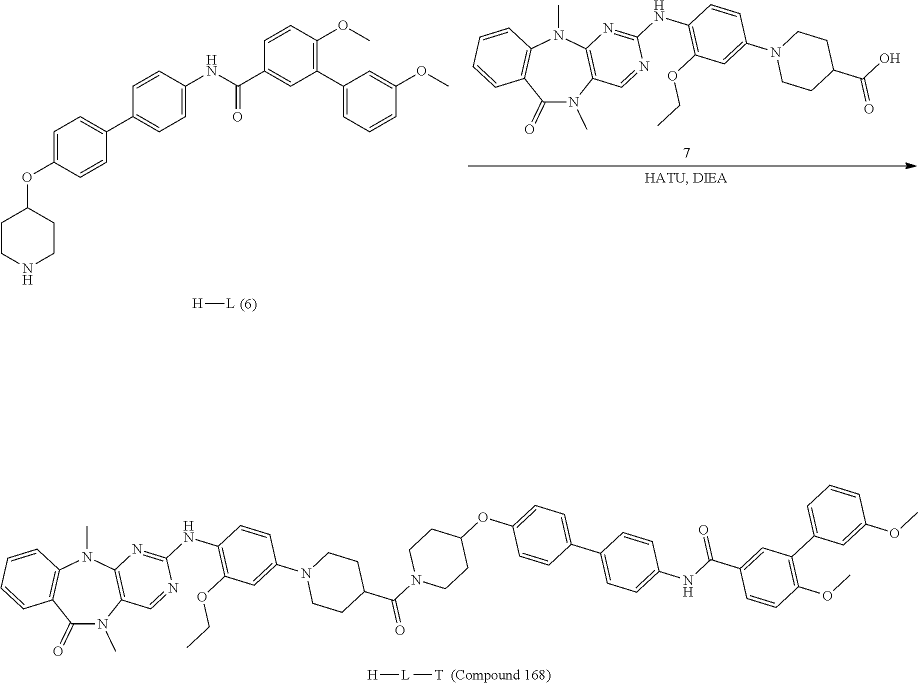

### Intermediate 3

To a solution of compound 1 (3.0 g, 16.20 mmol) and compound 2 (4.6 g, 17.82 mmol) in DMF (50 mL) was added DIEA (8.37 g, 64.79 mmol), followed by addition of HATU (6.77 g, 17.82 mmol). The mixture was stirred at room temperature for overnight. LC-MS indicated the reaction was completed. The reaction mixture was diluted with water (100 mL), extracted with EA (150 mL×2). The combined organic layer was washed saturated aqueous solution of NaHCO₃, concentrated under vacuum, the crude was purified by SGC eluted with DCM:MeOH=50:1 to give compound 3 (2.7 g, yield 39%) as a brown solid.

### Intermediate 5

To a solution of compound 3 (200 mg, 0.47 mmol), compound 4 (189.2 mg, 0.94 mmol) and PPh₃ (369.9 mg, 1.41 mmol) in dry THF (9 mL). The mixture was stirred at room temperature for 15 min under Ar atmosphere. The DEAD (245.6 mg, 1.41 mmol) was added. Then the reaction mixture was heated to 65° C. and stirred for overnight under Ar atmosphere. LC-MS indicated the reaction was completed. The reaction mixture was diluted with water (30 mL), extracted with EA (50 mL×2). The combined organic layer was concentrated under vacuum and purified by SGC eluted with PE:EA=3:1 to give compound 5 (152 mg, yield 53%) as a yellow solid.

### H-L (6)

The solution of compound 3 (152 mg, 0.25 mmol) in MeOH (2 mL) was added HCl/dioxane (2 mL). The mixture was stirred at room temperature for 1 h, LC-MS indicated the reaction was completed. The reaction mixture was concentrated under vacuum to give H-L (6, 124 mg, yield 97%) as a yellow solid.

### H-L-T (Compound 168)

### N-(4′-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)oxy)-[1,1′-biphenyl]-4-yl)-3′,6-dimethoxy-[1,1′-biphenyl]-3-carboxamide, trifluoroacetic acid

To a solution of H-L (6, 35.79 mg, 0.066 mmol) and compound 7 (30 mg, 0.06 mmol) in DMF (2 mL) was added DIEA (30.86 mg, 0.239 mmol), followed by addition of HATU (24.97 mg, 0.066 mmol). The mixture was stirred at room temperature for 1 h. LC-MS indicated the reaction was completed. The reaction mixture was purified by pre-HPLC(TFA) to give H-L-T (Compound 168, 18.92 mg, yield 32%) as a yellow solid. ¹H NMR (400 MHz, DMSO-d₆): δ 10.22 (s, 1H), 8.39 (s, 1H), 8.25-7.95 (m, 4H), 7.85 (d, J=8.4 Hz, 2H), 7.73-7.49 (m, 6H), 7.40-6.95 (m, 11H), 4.75-4.66 (m, 2H), 4.19-4.11 (m, 2H), 3.94-3.78 (m, 8H), 3.71-3.63 (m, 2H), 3.54-3.45 (m, 1H), 3.40 (s, 4H), 3.33 (s, 4H), 3.09-2.95 (m, 1H), 2.10-1.85 (m, 6H), 1.74-1.50 (m, 2H), 1.37 (t, J=6.8 Hz, 3H), 1.27-1.21 (m, 1H). LCMS (ESI): R_{T}=1.778 min, m/z found 993.1 [M-CF₃COOH+H]⁺.
Additional compounds were made according to the general procedure and scheme noted in the above Examples are listed in Table 1

### Example 4: Testing Various CHAMP Molecules

### Materials and Methods

### HSP90α-Binding Fluorescent Polarization (FP) Assay

Binding of test compounds to HSP90α protein was measured by fluorescent polarization (FP) using the HSP90α (N-terminal) Assay Kit (BPS Bioscience, #50298), following the manufacturer's instructions, except as noted. Fluorescently labeled HSP90-binding compounds, either the provided FITC-geldanamycin (5 nM final concentration) or RNK04010, a triazolone-based HSP90-binding small molecule labeled with BODIPY through a piperizine-phenyl linker (5 nM final concentration) were employed. A 2.5-fold serial dilution of each test compound ranging from 20 μM to 5.2 nM was assayed for binding to HSP90α. After the final step of adding HSP90α protein to each assay well, plates were mixed by brief shaking, incubated at 25° C. for 120 min for FITC-geldanamycin or 300 min for RNK04010, and fluorescence was measured using a PerkinElmer EnVision Plate Reader. Background-subtracted mP values were calculated from raw data and a four-parameter “log[inhibitor] vs. response” curve was fitted and IC50 values (the concentration at which 50% of the maximal inhibition occurs) calculated using GraphPad Prism 7 software.

### Results

A number of synthetic schemes have been developed to construct various CHAMP molecules designed to degrade a target protein or proteins. A representative example is shown consisting of a HSP90 binder linked to a target protein binder. Similar chemistry can be applied to other CHAMP molecules not limited to these specific HSP90- and target binding moieties.

HSP90α-binding fluorescent polarization (FP) assays measuring competition with the fluorescently labeled HSP90 binders, FITC-geldanamycin or RNK04010 (BODIPY-labeled), were applied to assess the binding capabilities of CHAMP molecules to HSP90. As shown in Table 2, CHAMP molecules containing HSP90-binding moieties documented in the literature were generally in agreement with the published structure activity relationship (SAR).

The incorporation of a target protein(s) binder of similar molecular weight to the HSP90 binder into the CHAMPs typically had only minimal impact on the binding of CHAMP molecules to HSP90α in this assay (Table 2). There are a number of reasons: first the co-crystal structures of these moieties with their corresponding proteins are available and allow precise structure-based molecular designs; and secondly, the linker is constructed to provide rigidity with suitable length.

TABLE 1
HL & HLT Compounds

| | HL | | HLT | |
|---|---|---|---|---|
| HL structure | Patent ID # | HLT structure | Patent ID # | M/Z |
| 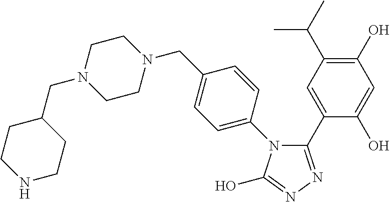 [SMILES: CC(C)c1cc(-c2nnc(O)n2-c2ccc(CN3CCN(CC4CCNCC4)CC3)cc2)c(O)cc1O] | 1 | 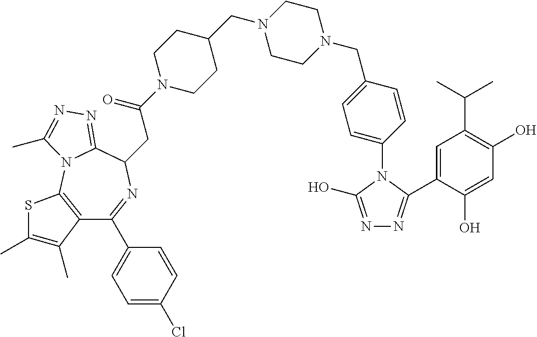 [SMILES: Cc1sc2c(c1C)C(c1ccc(Cl)cc1)=NC(CC(=O)N1CCC(CN3CCN(Cc4ccc(-n5c(O)nnc5-c5cc(C(C)C)c(O)cc5O)cc4)CC3)CC1)c1nnc(C)n1-2] | 1 | 889.6 [M − HCl + H]+ |
| 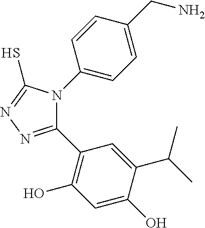 | 2 | 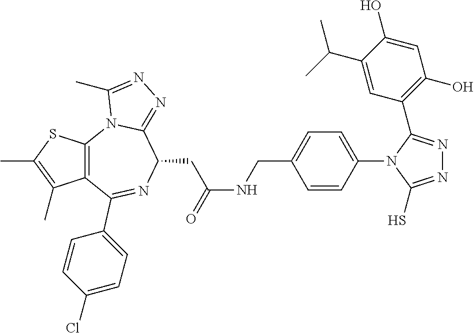 [SMILES: Cc1sc2c(c1C)C(c1ccc(Cl)cc1)=N[C@@H](CC(=O)NCc1ccc(-n3c(S)nnc3-c3cc(C(C)C)c(O)cc3O)cc1)c1nnc(C)n1-2] | 2 | 739.2 [M + H]+ |
| 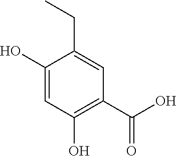 | 3 | | 3 | |
| 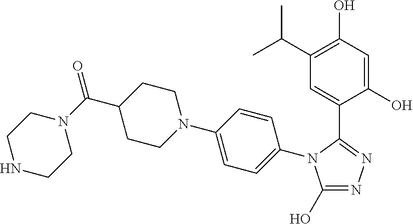 | 4 | 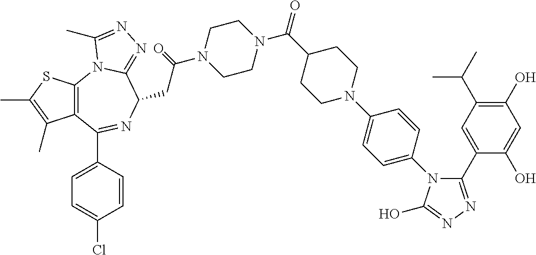 [SMILES: Cc1sc2c(c1C)C(c1ccc(Cl)cc1)=N[C@@H](CC(=O)N1CCN(C(=O)C3CCN(c4ccc(-n5c(O)nnc5-c5cc(C(C)C)c(O)cc5O)cc4)CC3)CC1)c1nnc(C)n1-2] | 4 | 889.5 [M + H]+ |
| 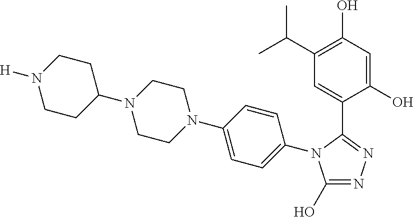 [SMILES: CC(C)c1cc(-c2nnc(O)n2-c2ccc(N3CCN(C4CCNCC4)CC3)cc2)c(O)cc1O] | 5 | 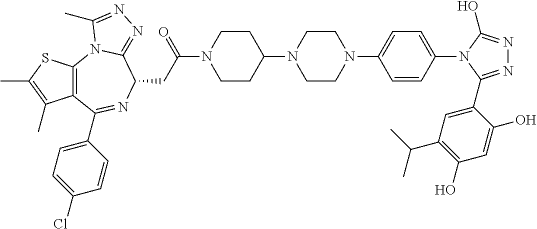 [SMILES: Cc1sc2c(c1C)C(c1ccc(Cl)cc1)=N[C@@H](CC(=O)N1CCC(N3CCN(c4ccc(-n5c(O)nnc5-c5cc(C(C)C)c(O)cc5O)cc4)CC3)CC1)c1nnc(C)n1-2] | 5 | 861.7 [M − CF3COOH + H]+ |
| 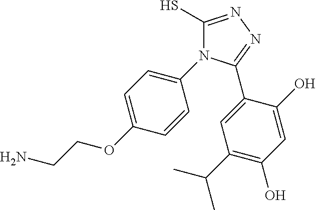 | 6 | 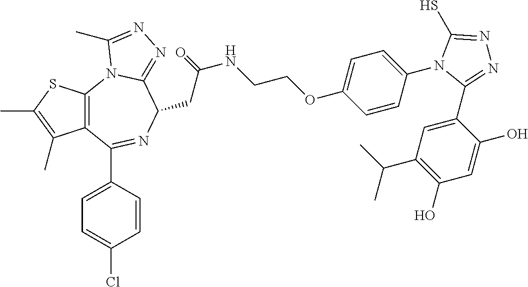 [SMILES: Cc1sc2c(c1C)C(c1ccc(Cl)cc1)=N[C@@H](CC(=O)NCCOc1ccc(-n3c(S)nnc3-c3cc(C(C)C)c(O)cc3O)cc1)c1nnc(C)n1-2] | 6 | 769.1 [M + H]+ |
| 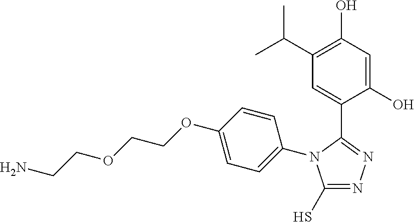 | 7 | 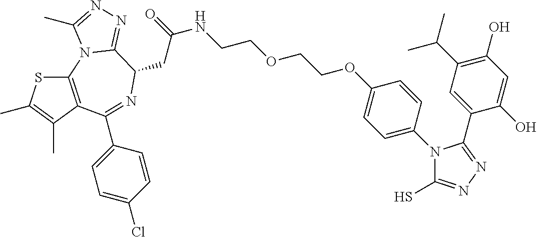 [SMILES: Cc1sc2c(c1C)C(c1ccc(Cl)cc1)=N[C@@H](CC(=O)NCCOCCOc1ccc(-n3c(S)nnc3-c3cc(C(C)C)c(O)cc3O)cc1)c1nnc(C)n1-2] | 7 | 813.2 [M + H]+ |
| 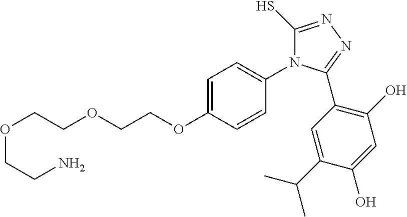 | 8 | 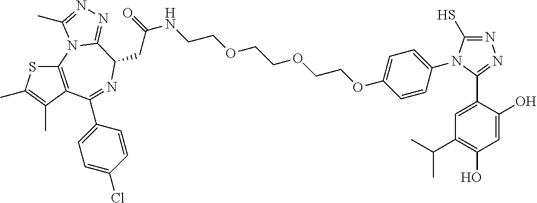 [SMILES: Cc1sc2c(c1C)C(c1ccc(Cl)cc1)=N[C@@H](CC(=O)NCCOCCOCCOc1ccc(-n3c(S)nnc3-c3cc(C(C)C)c(O)cc3O)cc1)c1nnc(C)n1-2] | 8 | 857.7 [M + H]+ |
| 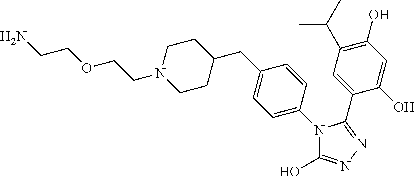 [SMILES: CC(C)c1cc(-c2nnc(O)n2-c2ccc(CC3CCN(CCOCCN)CC3)cc2)c(O)cc1O] | 9 | 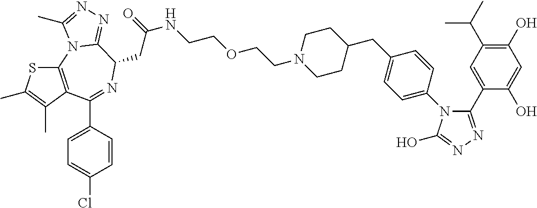 [SMILES: Cc1sc2c(c1C)C(c1ccc(Cl)cc1)=N[C@@H](CC(=O)NCCOCCN1CCC(Cc3ccc(-n4c(O)nnc4-c4cc(C(C)C)c(O)cc4O)cc3)CC1)c1nnc(C)n1-2] | 9 | 878.2 [M − CF3COOH + H]+ |
| 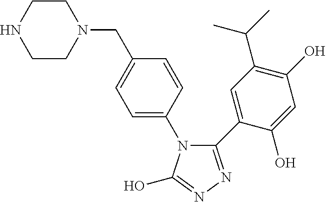 [SMILES: CC(C)c1cc(-c2nnc(O)n2-c2ccc(CN3CCNCC3)cc2)c(O)cc1O] | 10 | 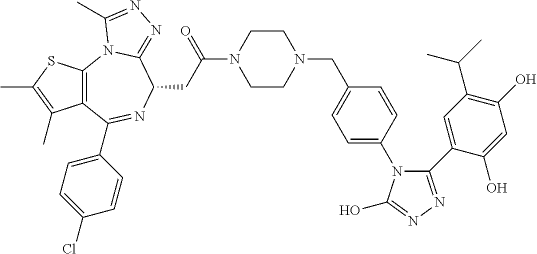 [SMILES: Cc1sc2c(c1C)C(c1ccc(Cl)cc1)=N[C@@H](CC(=O)N1CCN(Cc3ccc(-n4c(O)nnc4-c4cc(C(C)C)c(O)cc4O)cc3)CC1)c1nnc(C)n1-2] | 10A | 792.4 [M − CF3COOH + H] + |
| 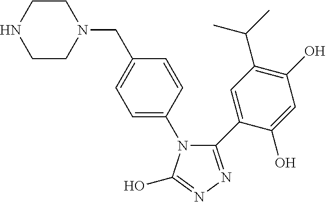 [SMILES: CC(C)c1cc(-c2nnc(O)n2-c2ccc(CN3CCNCC3)cc2)c(O)cc1O] | 10 | 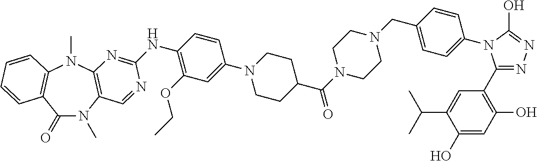 [SMILES: CCOc1cc(N2CCC(C(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C] | 10B | 894.3 [M − HCl + H]+ |
| 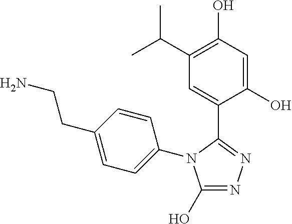 | 11 | 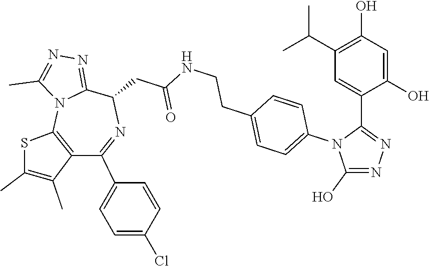 [SMILES: Cc1sc2c(c1C)C(c1ccc(Cl)cc1)=N[C@@H](CC(=O)NCCc1ccc(-n3c(O)nnc3-c3cc(C(C)C)c(O)cc3O)cc1)c1nnc(C)n1-2] | 11 | 737.6 [M + H]+). |
| 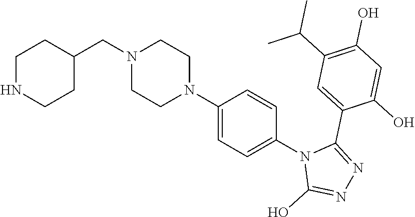 | 12 | 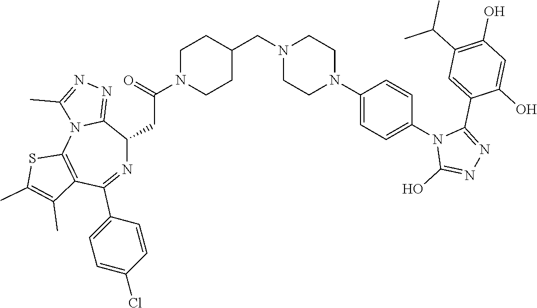 [SMILES: Cc1sc2c(c1C)C(c1ccc(Cl)cc1)=N[C@@H](CC(=O)N1CCC(CN3CCN(c4ccc(-n5c(O)nnc5-c5cc(C(C)C)c(O)cc5O)cc4)CC3)CC1)c1nnc(C)n1-2] | 12 | 875.7 [M + H]+ |
| 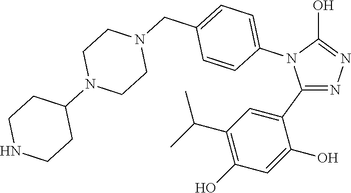 [SMILES: CC(C)c1cc(-c2nnc(O)n2-c2ccc(CN3CCN(C4CCNCC4)CC3)cc2)c(O)cc1O] | 13 | 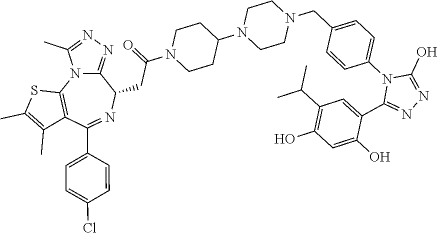 [SMILES: Cc1sc2c(c1C)C(c1ccc(Cl)cc1)=N[C@@H](CC(=O)N1CCC(N3CCN(Cc4ccc(-n5c(O)nnc5-c5cc(C(C)C)c(O)cc5O)cc4)CC3)CC1)c1nnc(C)n1-2] | 13 | 875.3 [M − CF3COOH + H]+ |
| 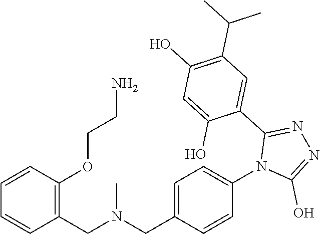 [SMILES: CC(C)c1cc(-c2nnc(O)n2-c2ccc(CN(C)Cc3ccccc3OCCN)cc2)c(O)cc1O] | 14 | 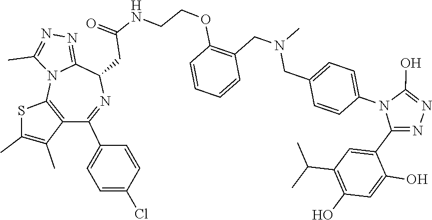 [SMILES: Cc1sc2c(c1C)C(c1ccc(Cl)cc1)=N[C@@H](CC(=O)NCCOc1ccccc1CN(C)Cc1ccc(-n3c(O)nnc3-c3cc(C(C)C)c(O)cc3O)cc1)c1nnc(C)n1-2] | 14 | 886.3 [M − CF3COOH + H]+ |
| 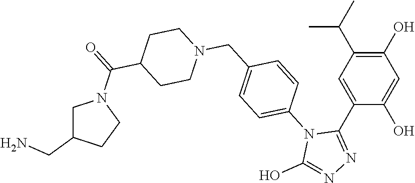 [SMILES: CC(C)c1cc(-c2nnc(O)n2-c2ccc(CN3CCC(C(=O)N4CCC(CN)C4)CC3)cc2)c(O)cc1O] | 15 | 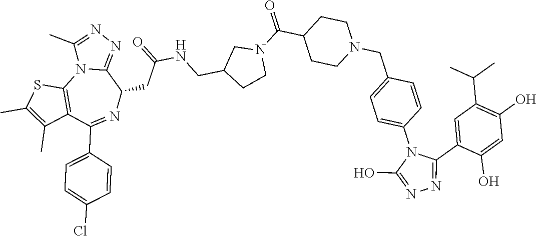 [SMILES: Cc1sc2c(c1C)C(c1ccc(Cl)cc1)=N[C@@H](CC(=O)NCC1CCN(C(=O)C3CCN(Cc4ccc(-n5c(O)nnc5-c5cc(C(C)C)c(O)cc5O)cc4)CC3)C1)c1nnc(C)n1-2] | 15 | 917.3 [M − CF3COOH + H]+ |
| 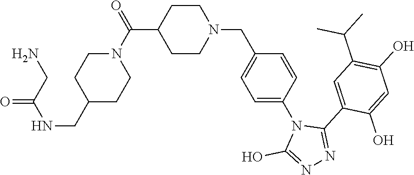 [SMILES: CC(C)c1cc(-c2nnc(O)n2-c2ccc(CN3CCC(C(=O)N4CCC(CNC(=O)CN)CC4)CC3)cc2)c(O)cc1O] | 16 | 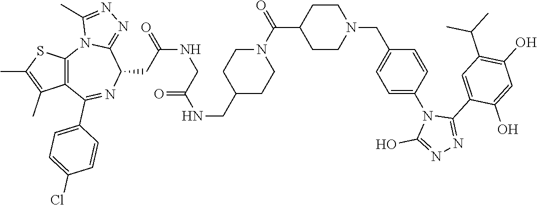 [SMILES: Cc1sc2c(c1C)C(c1ccc(Cl)cc1)=N[C@@H](CC(=O)NCC(=O)NCC1CCN(C(=O)C3CCN(Cc4ccc(-n5c(O)nnc5-c5cc(C(C)C)c(O)cc5O)cc4)CC3)CC1)c1nnc(C)n1-2] | 16 | 988.3 [M − CF3COOH + H]+ |
| 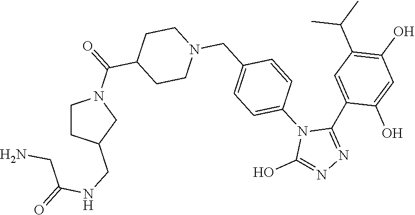 [SMILES: CC(C)c1cc(-c2nnc(O)n2-c2ccc(CN3CCC(C(=O)N4CCC(CNC(=O)CN)C4)CC3)cc2)c(O)cc1O] | 17 | 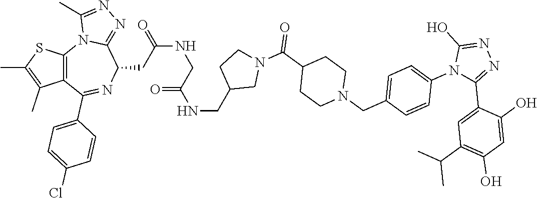 [SMILES: Cc1sc2c(c1C)C(c1ccc(Cl)cc1)=N[C@@H](CC(=O)NCC(=O)NCC1CCN(C(=O)C3CCN(Cc4ccc(-n5c(O)nnc5-c5cc(C(C)C)c(O)cc5O)cc4)CC3)C1)c1nnc(C)n1-2] | 17 | 974.3 [M − CF3COOH + H]+ |
| 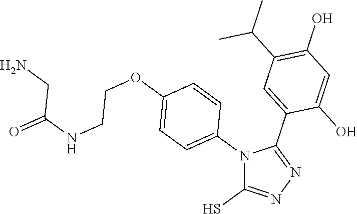 | 18 | 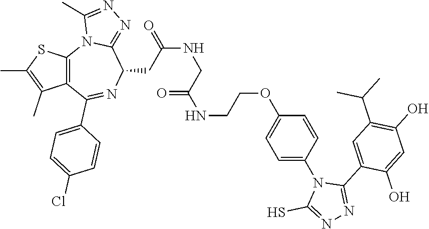 [SMILES: Cc1sc2c(c1C)C(c1ccc(Cl)cc1)=N[C@@H](CC(=O)NCC(=O)NCCOc1ccc(-n3c(S)nnc3-c3cc(C(C)C)c(O)cc3O)cc1)c1nnc(C)n1-2] | 18 | 826.2 [M + H]+ |
| 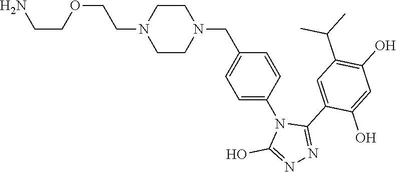 [SMILES: CC(C)c1cc(-c2nnc(O)n2-c2ccc(CN3CCN(CCOCCN)CC3)cc2)c(O)cc1O] | 19 | 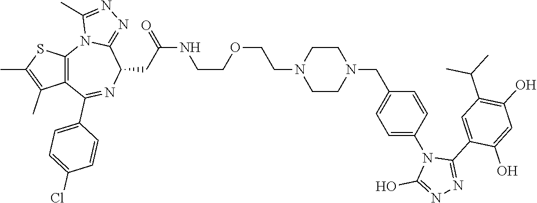 [SMILES: Cc1sc2c(c1C)C(c1ccc(Cl)cc1)=N[C@@H](CC(=O)NCCOCCN1CCN(Cc3ccc(-n4c(O)nnc4-c4cc(C(C)C)c(O)cc4O)cc3)CC1)c1nnc(C)n1-2] | 19 | 879.3 [M − CF3COOH + H]+ |
| 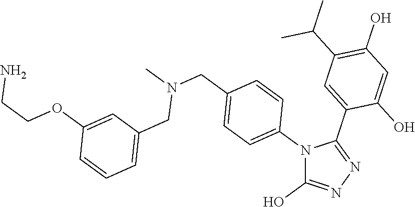 [SMILES: CC(C)c1cc(-c2nnc(O)n2-c2ccc(CN(C)Cc3cccc(OCCN)c3)cc2)c(O)cc1O] | 20 | 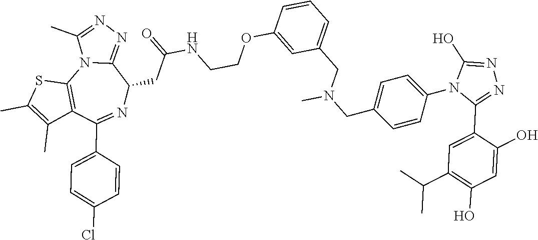 [SMILES: Cc1sc2c(c1C)C(c1ccc(Cl)cc1)=N[C@@H](CC(=O)NCCOc1cccc(CN(C)Cc3ccc(-n4c(O)nnc4-c4cc(C(C)C)c(O)cc4O)cc3)c1)c1nnc(C)n1-2] | 20 | 886.5 [M − CF3COOH + H]+ |
| 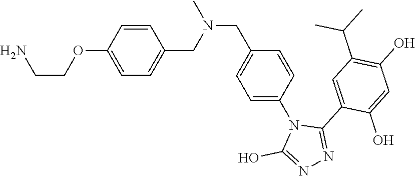 [SMILES: CC(C)c1cc(-c2nnc(O)n2-c2ccc(CN(C)Cc3ccc(OCCN)cc3)cc2)c(O)cc1O] | 21 | 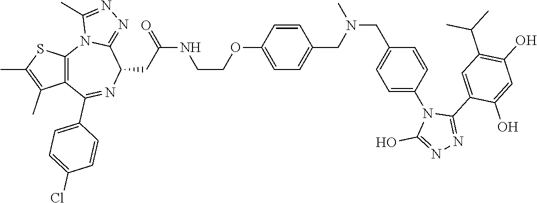 [SMILES: Cc1sc2c(c1C)C(c1ccc(Cl)cc1)=N[C@@H](CC(=O)NCCOc1ccc(CN(C)Cc3ccc(-n4c(O)nnc4-c4cc(C(C)C)c(O)cc4O)cc3)cc1)c1nnc(C)n1-2] | 21 | 886.2 [MCF3COOH + H]+ |
| 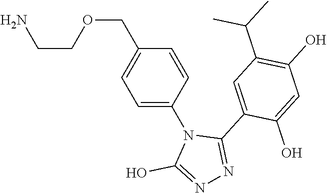 | 22 | 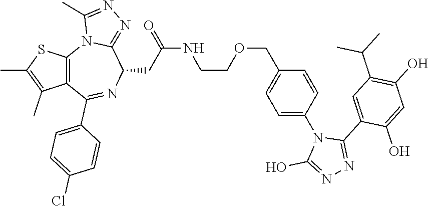 [SMILES: Cc1sc2c(c1C)C(c1ccc(Cl)cc1)=N[C@@H](CC(=O)NCCOCc1ccc(-n3c(O)nnc3-c3cc(C(C)C)c(O)cc3O)cc1)c1nnc(C)n1-2] | 22 | 767.2 [M + H]+ |
| 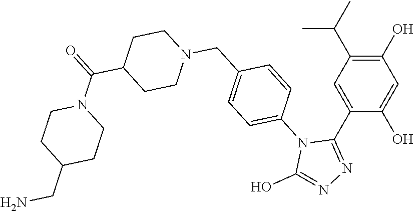 [SMILES: CC(C)c1cc(-c2nnc(O)n2-c2ccc(CN3CCC(C(=O)N4CCC(CN)CC4)CC3)cc2)c(O)cc1O] | 23 | 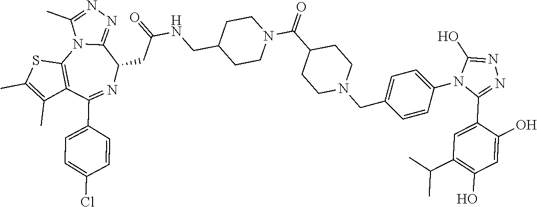 [SMILES: Cc1sc2c(c1C)C(c1ccc(Cl)cc1)=N[C@@H](CC(=O)NCC1CCN(C(=O)C3CCN(Cc4ccc(-n5c(O)nnc5-c5cc(C(C)C)c(O)cc5O)cc4)CC3)CC1)c1nnc(C)n1-2] | 23 | 931.7 [M − CF3COOH + H]+ |
| 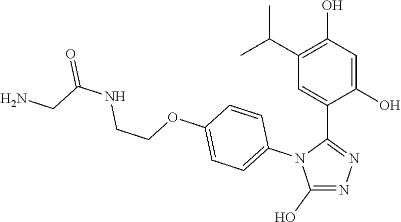 | 24 | 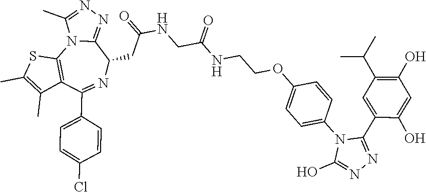 [SMILES: Cc1sc2c(c1C)C(c1ccc(Cl)cc1)=N[C@@H](CC(=O)NCC(=O)NCCOc1ccc(-n3c(O)nnc3-c3cc(C(C)C)c(O)cc3O)cc1)c1nnc(C)n1-2] | 24 | 810.6 [M + H]+ |
| 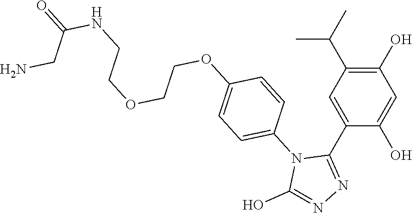 [SMILES: CC(C)c1cc(-c2nnc(O)n2-c2ccc(OCCOCCNC(=O)CN)cc2)c(O)cc1O] | 25 | 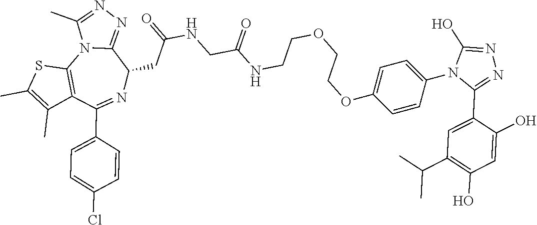 [SMILES: Cc1sc2c(c1C)C(c1ccc(Cl)cc1)=N[C@@H](CC(=O)NCC(=O)NCCOCCOc1ccc(-n3c(O)nnc3-c3cc(C(C)C)c(O)cc3O)cc1)c1nnc(C)n1-2] | 25 | 852.2 [M − H]− |
| 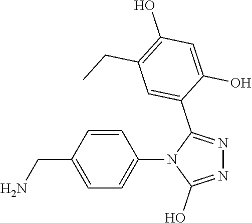 | 26 | 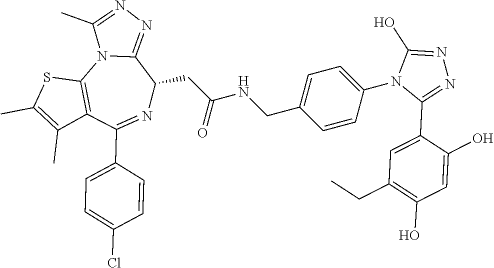 [SMILES: CCc1cc(-c2nnc(O)n2-c2ccc(CNC(=O)C[C@@H]3N=C(c4ccc(Cl)cc4)c4c(sc(C)c4C)-n4c(C)nnc43)cc2)c(O)cc1O] | 26 | 709.6 [M + H]+ |
| 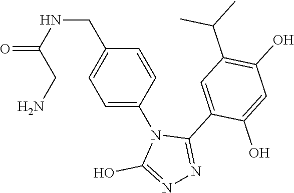 | 27 | 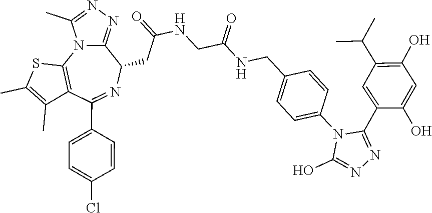 [SMILES: Cc1sc2c(c1C)C(c1ccc(Cl)cc1)=N[C@@H](CC(=O)NCC(=O)NCc1ccc(-n3c(O)nnc3-c3cc(C(C)C)c(O)cc3O)cc1)c1nnc(C)n1-2] | 27 | 780.6 [M + H]+ |
| 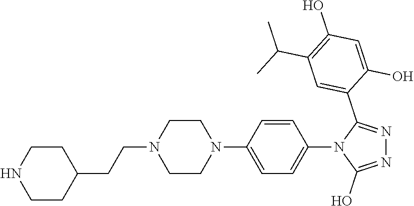 [SMILES: CC(C)c1cc(-c2nnc(O)n2-c2ccc(N3CCN(CCC4CCNCC4)CC3)cc2)c(O)cc1O] | 28 | 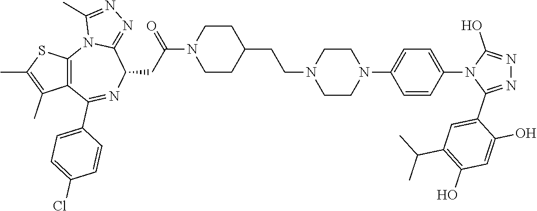 [SMILES: Cc1sc2c(c1C)C(c1ccc(Cl)cc1)=N[C@@H](CC(=O)N1CCC(CCN3CCN(c4ccc(-n5c(O)nnc5-c5cc(C(C)C)c(O)cc5O)cc4)CC3)CC1)c1nnc(C)n1-2] | 28 | 889.4 [M − CF3COOH + H]+ |
| 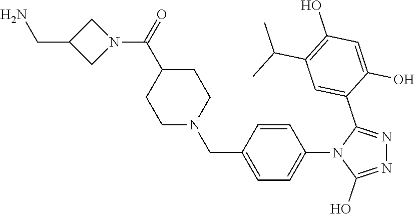 [SMILES: CC(C)c1cc(-c2nnc(O)n2-c2ccc(CN3CCC(C(=O)N4CC(CN)C4)CC3)cc2)c(O)cc1O] | 29 | 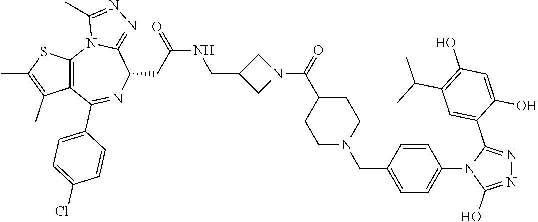 [SMILES: Cc1sc2c(c1C)C(c1ccc(Cl)cc1)=N[C@@H](CC(=O)NCC1CN(C(=O)C3CCN(Cc4ccc(-n5c(O)nnc5-c5cc(C(C)C)c(O)cc5O)cc4)CC3)C1)c1nnc(C)n1-2] | 29 | 903.4 [M − CF3COOH + H]+ |
| 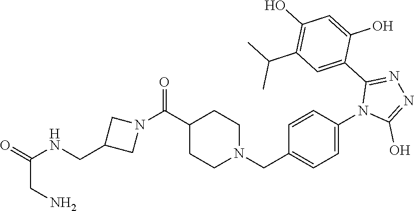 [SMILES: CC(C)c1cc(-c2nnc(O)n2-c2ccc(CN3CCC(C(=O)N4CC(CNC(=O)CN)C4)CC3)cc2)c(O)cc1O] | 30 | 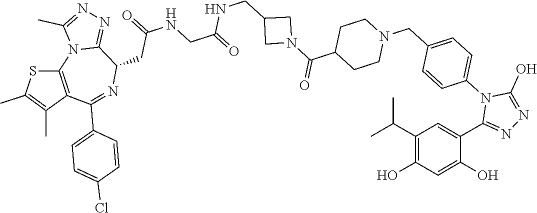 [SMILES: Cc1sc2c(c1C)C(c1ccc(Cl)cc1)=N[C@@H](CC(=O)NCC(=O)NCC1CN(C(=O)C3CCN(Cc4ccc(-n5c(O)nnc5-c5cc(C(C)C)c(O)cc5O)cc4)CC3)C1)c1nnc(C)n1-2] | 30 | 960.3 [M − CF3COOH + H]+ |
| 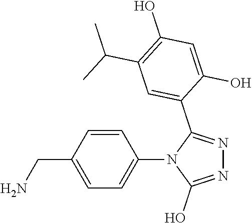 [SMILES: CC(C)c1cc(-c2nnc(O)n2-c2ccc(CN)cc2)c(O)cc1O] | 31 | 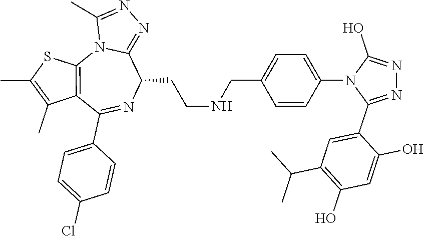 [SMILES: Cc1sc2c(c1C)C(c1ccc(Cl)cc1)=N[C@@H](CCNCc1ccc(-n3c(O)nnc3-c3cc(C(C)C)c(O)cc3O)cc1)c1nnc(C)n1-2] | 31 | 709.2 [M − CF3COOH + H]+ |
| 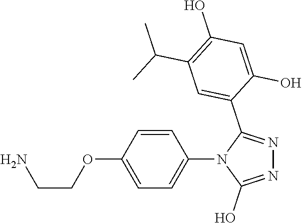 [SMILES: CC(C)c1cc(-c2nnc(O)n2-c2ccc(OCCN)cc2)c(O)cc1O] | 32 | 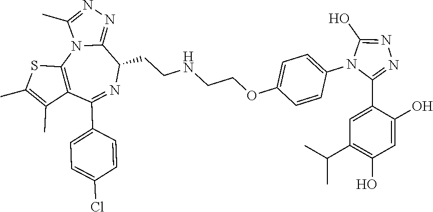 [SMILES: Cc1sc2c(c1C)C(c1ccc(Cl)cc1)=N[C@@H](CCNCCOc1ccc(-n3c(O)nnc3-c3cc(C(C)C)c(O)cc3O)cc1)c1nnc(C)n1-2] | 32 | 739.2 [M − CF3COOH + H]+ |
| 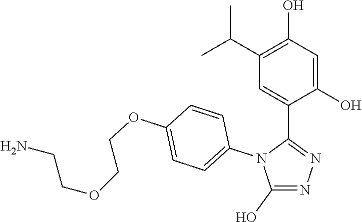 [SMILES: CC(C)c1cc(-c2nnc(O)n2-c2ccc(OCCOCCN)cc2)c(O)cc1O] | 33 | 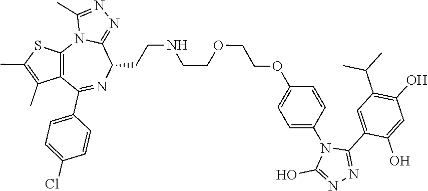 [SMILES: Cc1sc2c(c1C)C(c1ccc(Cl)cc1)=N[C@@H](CCNCCOCCOc1ccc(-n3c(O)nnc3-c3cc(C(C)C)c(O)cc3O)cc1)c1nnc(C)n1-2] | 33 | 783.5 [M − CF3COOH + H]+ |
| 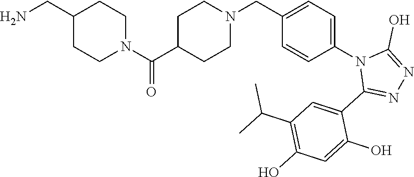 [SMILES: CC(C)c1cc(-c2nnc(O)n2-c2ccc(CN3CCC(C(=O)N4CCC(CN)CC4)CC3)cc2)c(O)cc1O] | 34 | 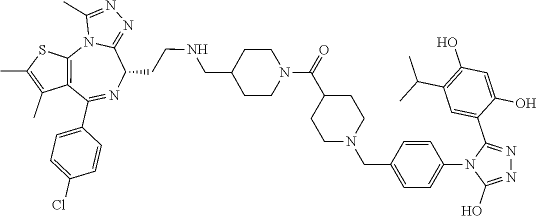 [SMILES: Cc1sc2c(c1C)C(c1ccc(Cl)cc1)=N[C@@H](CCNCC1CCN(C(=O)C3CCN(Cc4ccc(-n5c(O)nnc5-c5cc(C(C)C)c(O)cc5O)cc4)CC3)CC1)c1nnc(C)n1-2] | 34 | 917.3 [M − CF3COOH + H]+ |
| 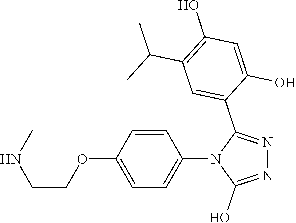 | 35 | 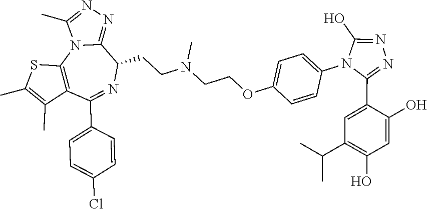 [SMILES: Cc1sc2c(c1C)C(c1ccc(Cl)cc1)=N[C@@H](CCN(C)CCOc1ccc(-n3c(O)nnc3-c3cc(C(C)C)c(O)cc3O)cc1)c1nnc(C)n1-2] | 35 | 753.7 [M + H]+ |
| 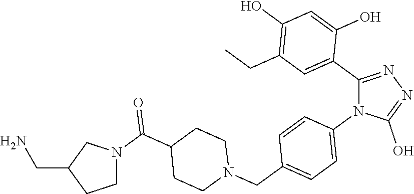 [SMILES: CCc1cc(-c2nnc(O)n2-c2ccc(CN3CCC(C(=O)N4CCC(CN)C4)CC3)cc2)c(O)cc1O] | 36 | 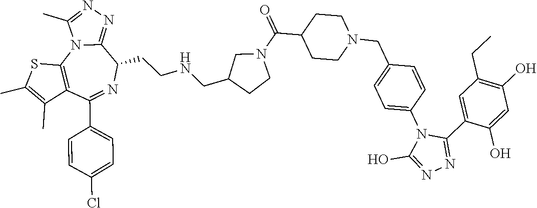 [SMILES: CCc1cc(-c2nnc(O)n2-c2ccc(CN3CCC(C(=O)N4CCC(CNCC[C@@H]5N=C(c6ccc(Cl)cc6)c6c(sc(C)c6C)-n6c(C)nnc65)C4)CC3)cc2)c(O)cc1O] | 36 | 889.3 [M − CF3COOH + H]+ |
| 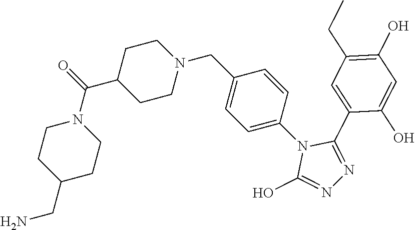 [SMILES: CCc1cc(-c2nnc(O)n2-c2ccc(CN3CCC(C(=O)N4CCC(CN)CC4)CC3)cc2)c(O)cc1O] | 37 | 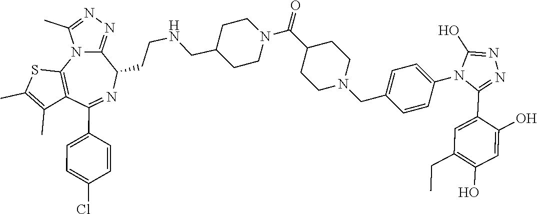 [SMILES: CCc1cc(-c2nnc(O)n2-c2ccc(CN3CCC(C(=O)N4CCC(CNCC[C@@H]5N=C(c6ccc(Cl)cc6)c6c(sc(C)c6C)-n6c(C)nnc65)CC4)CC3)cc2)c(O)cc1O] | 37 | 903.3 [M − CF3COOH + H]+ |
| 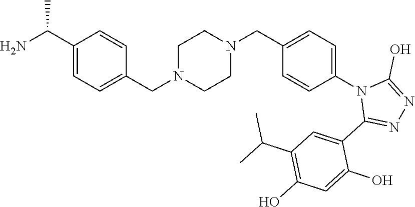 [SMILES: CC(C)c1cc(-c2nnc(O)n2-c2ccc(CN3CCN(Cc4ccc([C@@H](C)N)cc4)CC3)cc2)c(O)cc1O] | 38 | 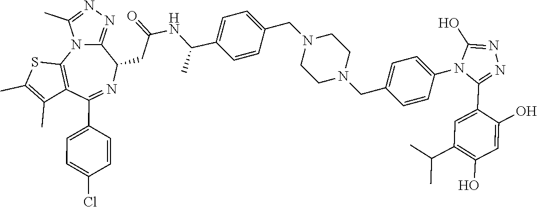 [SMILES: Cc1sc2c(c1C)C(c1ccc(Cl)cc1)=N[C@@H](CC(=O)N[C@@H](C)c1ccc(CN3CCN(Cc4ccc(-n5c(O)nnc5-c5cc(C(C)C)c(O)cc5O)cc4)CC3)cc1)c1nnc(C)n1-2] | 38 | 925.5 [M − CF3COOH + H] + |
| 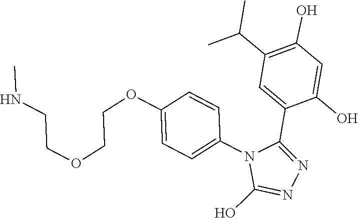 [SMILES: CNCCOCCOc1ccc(-n2c(O)nnc2-c2cc(C(C)C)c(O)cc2O)cc1] | 39 | 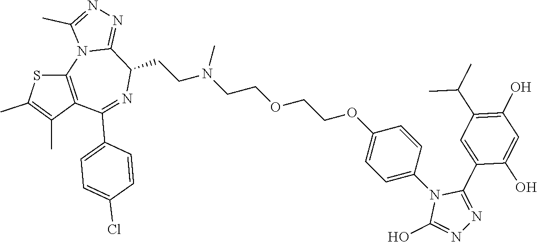 [SMILES: Cc1sc2c(c1C)C(c1ccc(Cl)cc1)=N[C@@H](CCN(C)CCOCCOc1ccc(-n3c(O)nnc3-c3cc(C(C)C)c(O)cc3O)cc1)c1nnc(C)n1-2] | 39 | 797.3 [M − CF3COOH + H]+ |
| 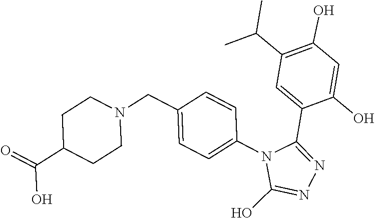 [SMILES: CC(C)c1cc(-c2nnc(O)n2-c2ccc(CN3CCC(C(=O)O)CC3)cc2)c(O)cc1O] | 40 | 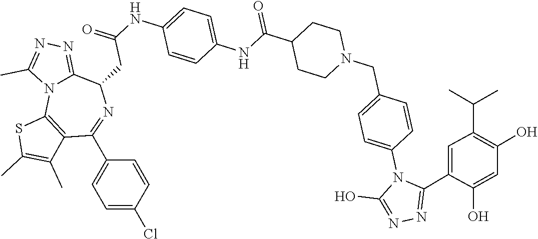 [SMILES: Cc1sc2c(c1C)C(c1ccc(Cl)cc1)=N[C@@H](CC(=O)Nc1ccc(NC(=O)C3CCN(Cc4ccc(-n5c(O)nnc5-c5cc(C(C)C)c(O)cc5O)cc4)CC3)cc1)c1nnc(C)n1-2] | 40 | 925.9 [M − CF3COOH + H]+ |
| 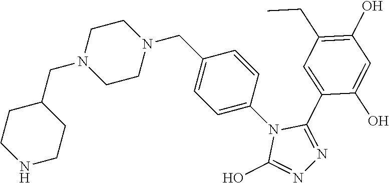 | 41 | 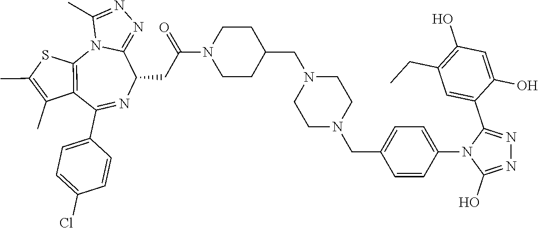 [SMILES: CCc1cc(-c2nnc(O)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 41 | 875.2 [M + H]+ |
| 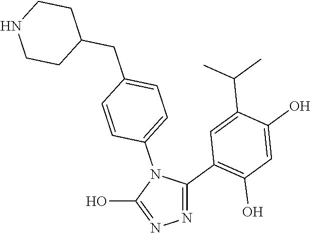 [SMILES: CC(C)c1cc(-c2nnc(O)n2-c2ccc(CC3CCNCC3)cc2)c(O)cc1O] | 42 | 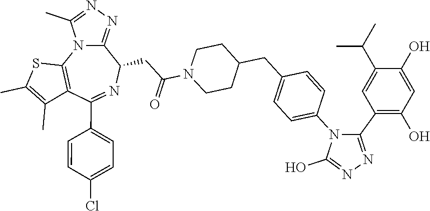 [SMILES: Cc1sc2c(c1C)C(c1ccc(Cl)cc1)=N[C@@H](CC(=O)N1CCC(Cc3ccc(-n4c(O)nnc4-c4cc(C(C)C)c(O)cc4O)cc3)CC1)c1nnc(C)n1-2] | 42 | 791.2 [MCF3COOH + H]+ |
| 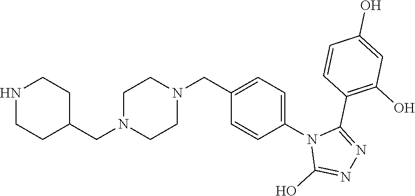 [SMILES: Oc1ccc(-c2nnc(O)n2-c2ccc(CN3CCN(CC4CCNCC4)CC3)cc2)c(O)c1] | 43 | 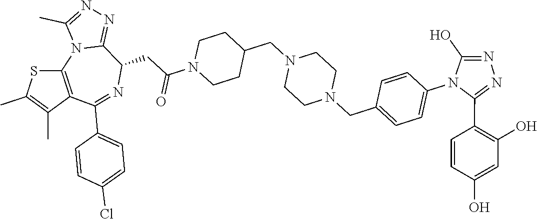 [SMILES: Cc1sc2c(c1C)C(c1ccc(Cl)cc1)=N[C@@H](CC(=O)N1CCC(CN3CCN(Cc4ccc(-n5c(O)nnc5-c5ccc(O)cc5O)cc4)CC3)CC1)c1nnc(C)n1-2] | 43 | 845.7 [M − CF3COOH − H]− |
| 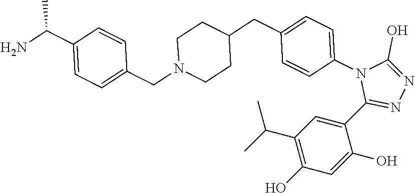 [SMILES: CC(C)c1cc(-c2nnc(O)n2-c2ccc(CC3CCN(Cc4ccc([C@@H](C)N)cc4)CC3)cc2)c(O)cc1O] | 44 | 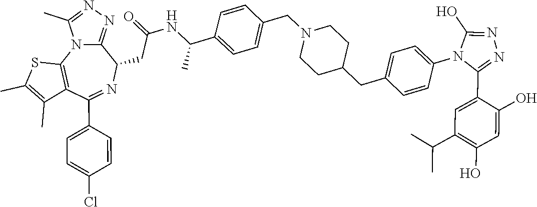 [SMILES: Cc1sc2c(c1C)C(c1ccc(Cl)cc1)=N[C@@H](CC(=O)N[C@@H](C)c1ccc(CN3CCC(Cc4ccc(-n5c(O)nnc5-c5cc(C(C)C)c(O)cc5O)cc4)CC3)cc1)c1nnc(C)n1-2] | 44 | 924 [M − CF3COOH + H]+ |
| 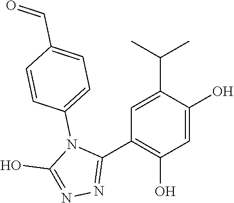 [SMILES: CC(C)c1cc(-c2nnc(O)n2-c2ccc(C=O)cc2)c(O)cc1O] | 45 | | 45 | |
| 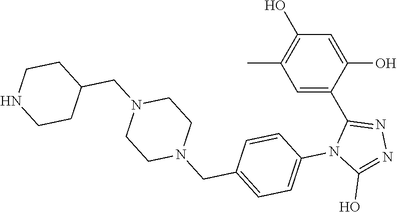 [SMILES: Cc1cc(-c2nnc(O)n2-c2ccc(CN3CCN(CC4CCNCC4)CC3)cc2)c(O)cc1O] | 46 | 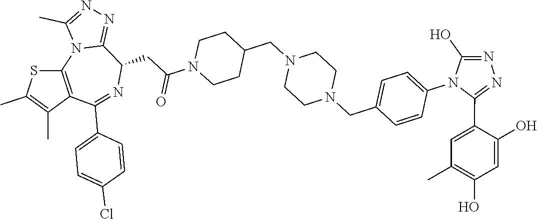 [SMILES: Cc1cc(-c2nnc(O)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 46 | 861.4 [M − CF3COOH + H]+ |
| 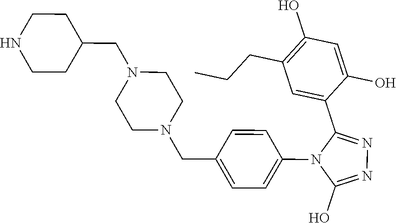 [SMILES: CCCc1cc(-c2nnc(O)n2-c2ccc(CN3CCN(CC4CCNCC4)CC3)cc2)c(O)cc1O] | 47 | 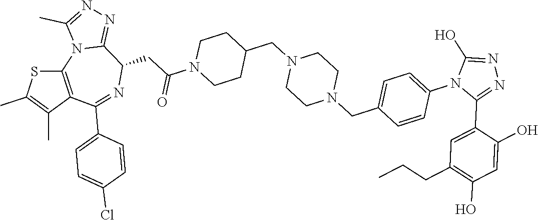 [SMILES: CCCc1cc(-c2nnc(O)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 47 | 889.3 [M − CF3COOH + H]+ |
| 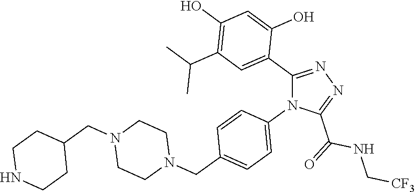 | 48 | 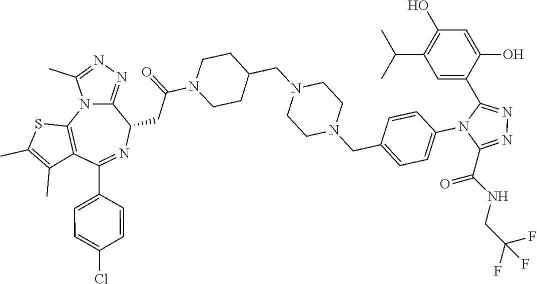 [SMILES: Cc1sc2c(c1C)C(c1ccc(Cl)cc1)=N[C@@H](CC(=O)N1CCC(CN3CCN(Cc4ccc(-n5c(C(=O)NCC(F)(F)F)nnc5-c5cc(C(C)C)c(O)cc5O)cc4)CC3)CC1)c1nnc(C)n1-2] | 48 | 998.3 [M + H]+ |
| 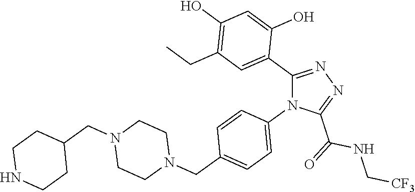 [SMILES: CCc1cc(-c2nnc(C(=O)NCC(F)(F)F)n2-c2ccc(CN3CCN(CC4CCNCC4)CC3)cc2)c(O)cc1O] | 49 | 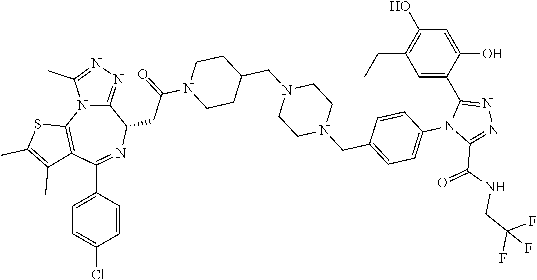 [SMILES: CCc1cc(-c2nnc(C(=O)NCC(F)(F)F)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 49 | 984.1 [M − CF3COOH + H]+ |
| 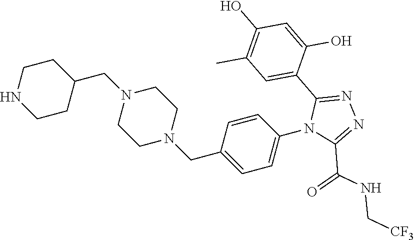 [SMILES: Cc1cc(-c2nnc(C(=O)NCC(F)(F)F)n2-c2ccc(CN3CCN(CC4CCNCC4)CC3)cc2)c(O)cc1O] | 50 | 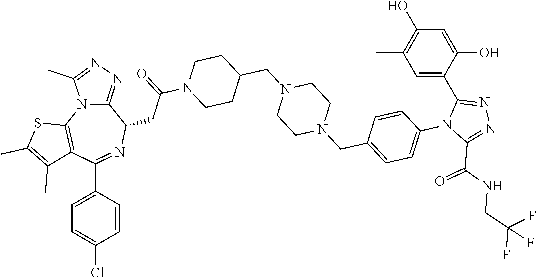 [SMILES: Cc1cc(-c2nnc(C(=O)NCC(F)(F)F)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 50 | 970.1 [MCF3COOH + H]+ |
| 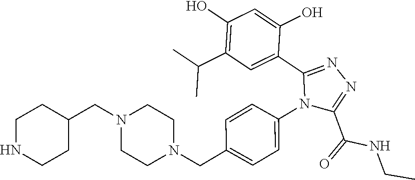 [SMILES: CCNC(=O)c1nnc(-c2cc(C(C)C)c(O)cc2O)n1-c1ccc(CN2CCN(CC3CCNCC3)CC2)cc1] | 51 | 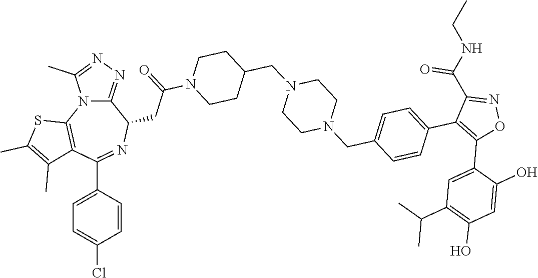 [SMILES: CCNC(=O)c1noc(-c2cc(C(C)C)c(O)cc2O)c1-c1ccc(CN2CCN(CC3CCN(C(=O)C[C@@H]4N=C(c5ccc(Cl)cc5)c5c(sc(C)c5C)-n5c(C)nnc54)CC3)CC2)cc1] | 51 | 944.6 [M − CF3COOH + H]+ |
| 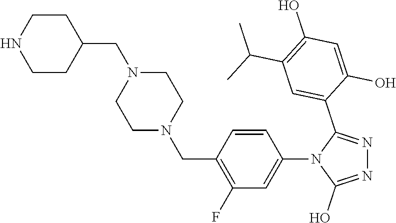 [SMILES: CC(C)c1cc(-c2nnc(O)n2-c2ccc(CN3CCN(CC4CCNCC4)CC3)c(F)c2)c(O)cc1O] | 52 | 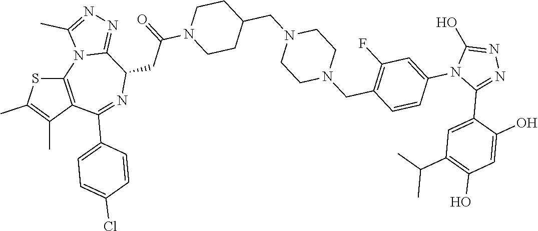 [SMILES: Cc1sc2c(c1C)C(c1ccc(Cl)cc1)=N[C@@H](CC(=O)N1CCC(CN3CCN(Cc4ccc(-n5c(O)nnc5-c5cc(C(C)C)c(O)cc5O)cc4F)CC3)CC1)c1nnc(C)n1-2] | 52 | 907.4 [M − CF3COOH + H]+ |
| 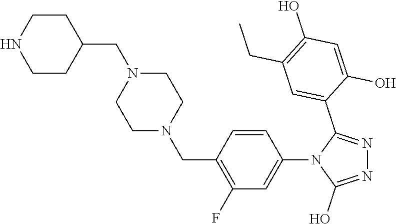 [SMILES: CCc1cc(-c2nnc(O)n2-c2ccc(CN3CCN(CC4CCNCC4)CC3)c(F)c2)c(O)cc1O] | 53 | 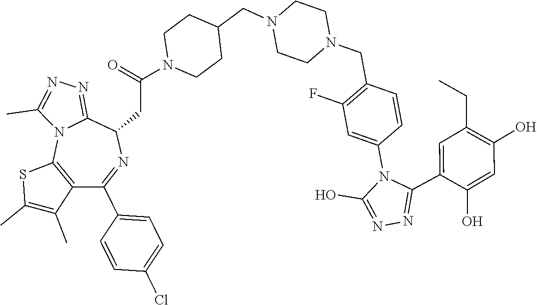 [SMILES: CCc1cc(-c2nnc(O)n2-c2ccc(CN3CCN(CC4CCN(C(=O)C[C@@H]5N=C(c6ccc(Cl)cc6)c6c(sc(C)c6C)-n6c(C)nnc65)CC4)CC3)c(F)c2)c(O)cc1O] | 53 | 893.1 [M − CF3COOH + H]+ |
| 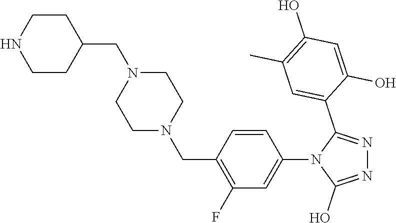 [SMILES: Cc1cc(-c2nnc(O)n2-c2ccc(CN3CCN(CC4CCNCC4)CC3)c(F)c2)c(O)cc1O] | 54 | 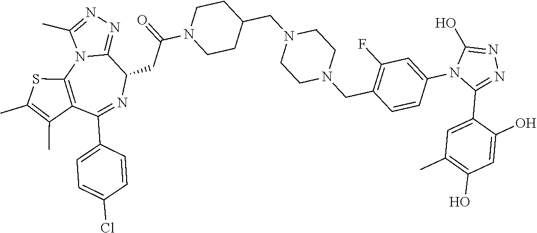 [SMILES: Cc1cc(-c2nnc(O)n2-c2ccc(CN3CCN(CC4CCN(C(=O)C[C@@H]5N=C(c6ccc(Cl)cc6)c6c(sc(C)c6C)-n6c(C)nnc65)CC4)CC3)c(F)c2)c(O)cc1O] | 54 | 877.2 [M − H]− |
| 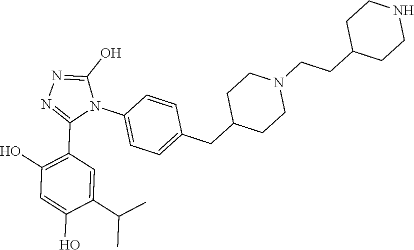 [SMILES: CC(C)c1cc(-c2nnc(O)n2-c2ccc(CC3CCN(CCC4CCNCC4)CC3)cc2)c(O)cc1O] | 55 | 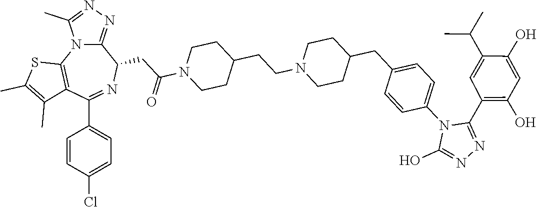 [SMILES: Cc1sc2c(c1C)C(c1ccc(Cl)cc1)=N[C@@H](CC(=O)N1CCC(CCN3CCC(Cc4ccc(-n5c(O)nnc5-c5cc(C(C)C)c(O)cc5O)cc4)CC3)CC1)c1nnc(C)n1-2] | 55 | 902.2 [M − CF3COOH + H]+ |
| 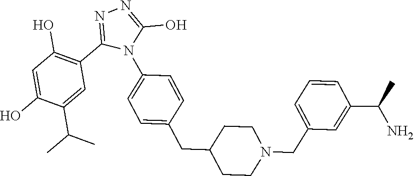 [SMILES: CC(C)c1cc(-c2nnc(O)n2-c2ccc(CC3CCN(Cc4cccc([C@@H](C)N)c4)CC3)cc2)c(O)cc1O] | 56 | 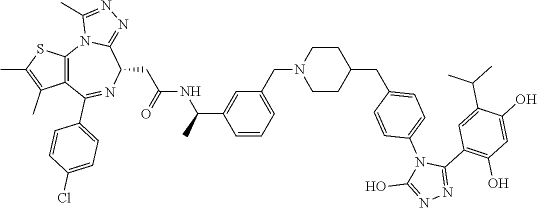 [SMILES: Cc1sc2c(c1C)C(c1ccc(Cl)cc1)=N[C@@H](CC(=O)N[C@H](C)c1cccc(CN3CCC(Cc4ccc(-n5c(O)nnc5-c5cc(C(C)C)c(O)cc5O)cc4)CC3)c1)c1nnc(C)n1-2] | 56 | 924.3 [M − CF3COOH + H]+ |
| 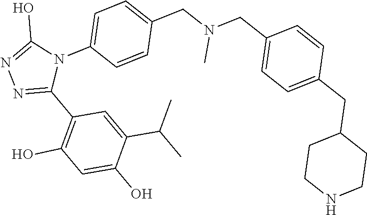 [SMILES: CC(C)c1cc(-c2nnc(O)n2-c2ccc(CN(C)Cc3ccc(CC4CCNCC4)cc3)cc2)c(O)cc1O] | 57 | 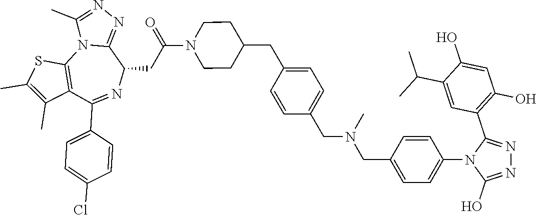 [SMILES: Cc1sc2c(c1C)C(c1ccc(Cl)cc1)=N[C@@H](CC(=O)N1CCC(Cc3ccc(CN(C)Cc4ccc(-n5c(O)nnc5-c5cc(C(C)C)c(O)cc5O)cc4)cc3)CC1)c1nnc(C)n1-2] | 57 | 924.2 [M − CF3COOH + H]+ |
| 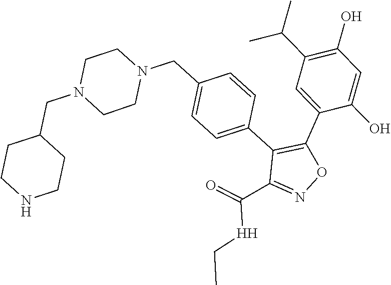 | 58 | 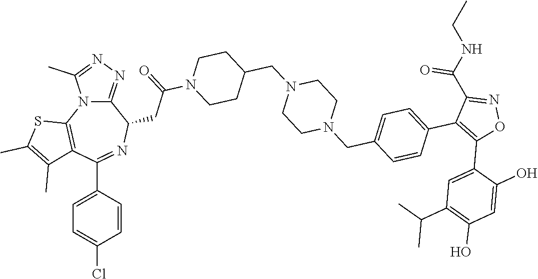 [SMILES: CCNC(=O)c1noc(-c2cc(C(C)C)c(O)cc2O)c1-c1ccc(CN2CCN(CC3CCN(C(=O)C[C@@H]4N=C(c5ccc(Cl)cc5)c5c(sc(C)c5C)-n5c(C)nnc54)CC3)CC2)cc1] | 58 | 944.2 [M − HCl + H]+ |
| 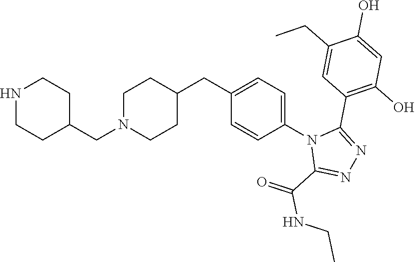 [SMILES: CCNC(=O)c1nnc(-c2cc(CC)c(O)cc2O)n1-c1ccc(CC2CCN(CC3CCNCC3)CC2)cc1] | 59 | 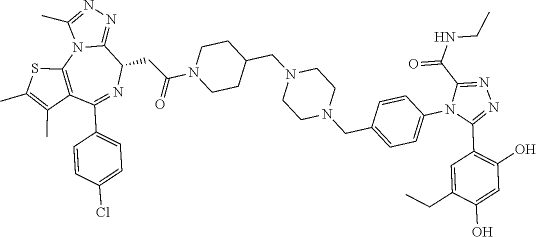 [SMILES: CCNC(=O)c1nnc(-c2cc(CC)c(O)cc2O)n1-c1ccc(CN2CCN(CC3CCN(C(=O)C[C@@H]4N=C(c5ccc(Cl)cc5)c5c(sc(C)c5C)-n5c(C)nnc54)CC3)CC2)cc1] | 59 | 929.3 [M − CF3COOH + H]+ |
| 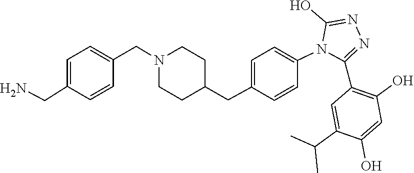 [SMILES: CC(C)c1cc(-c2nnc(O)n2-c2ccc(CC3CCN(Cc4ccc(CN)cc4)CC3)cc2)c(O)cc1O] | 60 | 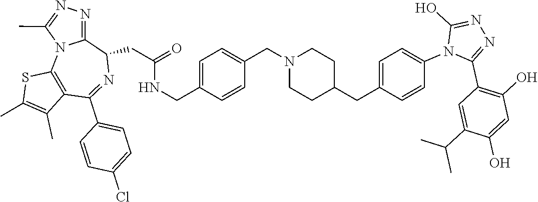 [SMILES: Cc1sc2c(c1C)C(c1ccc(Cl)cc1)=N[C@@H](CC(=O)NCc1ccc(CN3CCC(Cc4ccc(-n5c(O)nnc5-c5cc(C(C)C)c(O)cc5O)cc4)CC3)cc1)c1nnc(C)n1-2] | 60 | 910.2 [M − CF3COOH + H]+ |
| 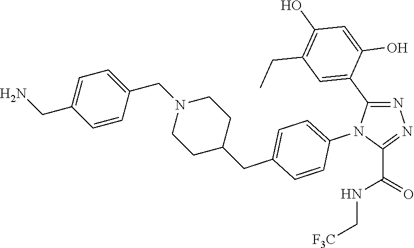 [SMILES: CCc1cc(-c2nnc(C(=O)NCC(F)(F)F)n2-c2ccc(CC3CCN(Cc4ccc(CN)cc4)CC3)cc2)c(O)cc1O] | 61 | 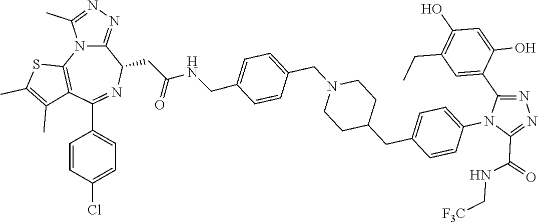 [SMILES: CCc1cc(-c2nnc(C(=O)NCC(F)(F)F)n2-c2ccc(CC3CCN(Cc4ccc(CNC(=O)C[C@@H]5N=C(c6ccc(Cl)cc6)c6c(sc(C)c6C)-n6c(C)nnc65)cc4)CC3)cc2)c(O)cc1O] | 61 | 889.6 [M − HCl + H]+ |
| 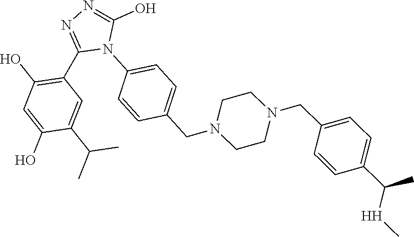 | 62 | 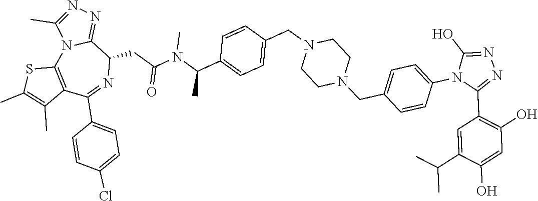 [SMILES: Cc1sc2c(c1C)C(c1ccc(Cl)cc1)=N[C@@H](CC(=O)N(C)[C@H](C)c1ccc(CN3CCN(Cc4ccc(-n5c(O)nnc5-c5cc(C(C)C)c(O)cc5O)cc4)CC3)cc1)c1nnc(C)n1-2] | 62 | 889.6 [M − HCl + H]+ |
| 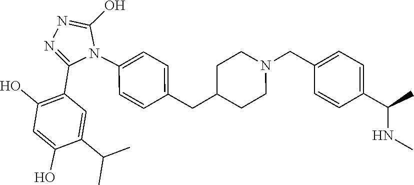 [SMILES: CN[C@H](C)c1ccc(CN2CCC(Cc3ccc(-n4c(O)nnc4-c4cc(C(C)C)c(O)cc4O)cc3)CC2)cc1] | 63 | 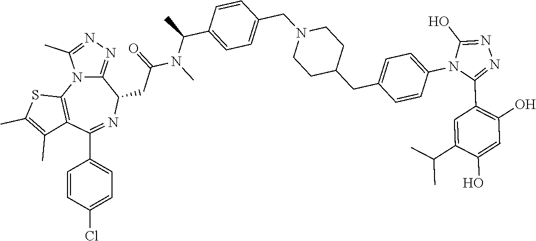 [SMILES: Cc1sc2c(c1C)C(c1ccc(Cl)cc1)=N[C@@H](CC(=O)N(C)[C@@H](C)c1ccc(CN3CCC(Cc4ccc(-n5c(O)nnc5-c5cc(C(C)C)c(O)cc5O)cc4)CC3)cc1)c1nnc(C)n1-2] | 63 | 999.4 [M − CF3COOH + H]+ |
| 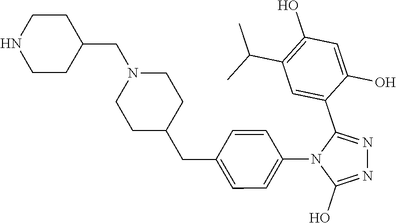 [SMILES: CC(C)c1cc(-c2nnc(O)n2-c2ccc(CC3CCN(CC4CCNCC4)CC3)cc2)c(O)cc1O] | 64 | 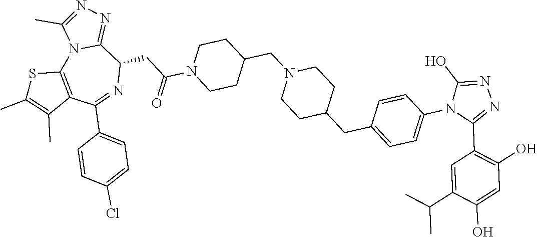 [SMILES: Cc1sc2c(c1C)C(c1ccc(Cl)cc1)=N[C@@H](CC(=O)N1CCC(CN3CCC(Cc4ccc(-n5c(O)nnc5-c5cc(C(C)C)c(O)cc5O)cc4)CC3)CC1)c1nnc(C)n1-2] | 64 | 1027.5 [M − CF3COOH + H]+ |
| 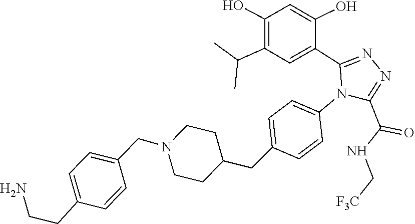 | 65 | 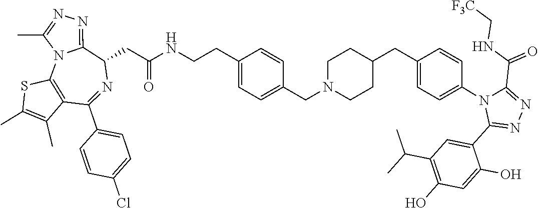 [SMILES: Cc1sc2c(c1C)C(c1ccc(Cl)cc1)=N[C@@H](CC(=O)NCCc1ccc(CN3CCC(Cc4ccc(-n5c(C(=O)NCC(F)(F)F)nnc5-c5cc(C(C)C)c(O)cc5O)cc4)CC3)cc1)c1nnc(C)n1-2] | 65 | 1033.3 [M − CF3COOH + H]+ |
| 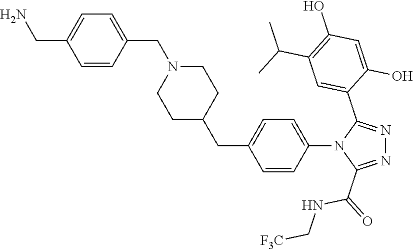 [SMILES: CC(C)c1cc(-c2nnc(C(=O)NCC(F)(F)F)n2-c2ccc(CC3CCN(Cc4ccc(CN)cc4)CC3)cc2)c(O)cc1O] | 66 | 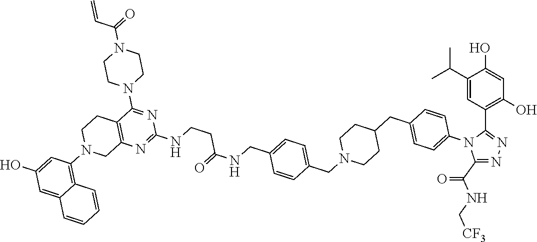 [SMILES: C=CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1] | 66 | 1121.5 [M − CF3COOH + H]+ |
| 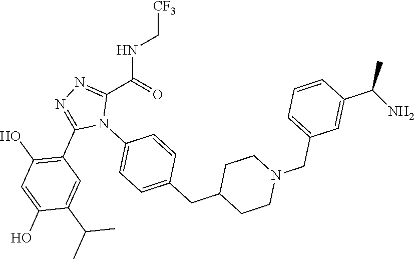 [SMILES: CC(C)c1cc(-c2nnc(C(=O)NCC(F)(F)F)n2-c2ccc(CC3CCN(Cc4cccc([C@@H](C)N)c4)CC3)cc2)c(O)cc1O] | 67 | 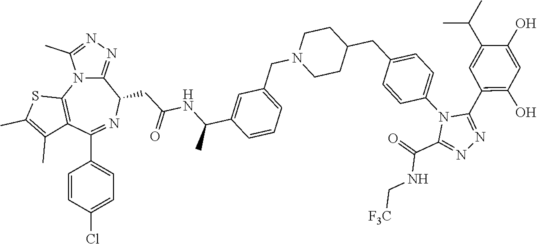 [SMILES: Cc1sc2c(c1C)C(c1ccc(Cl)cc1)=N[C@@H](CC(=O)N[C@H](C)c1cccc(CN3CCC(Cc4ccc(-n5c(C(=O)NCC(F)(F)F)nnc5-c5cc(C(C)C)c(O)cc5O)cc4)CC3)c1)c1nnc(C)n1-2] | 67 | 902.2 [M − HCl + H]+ |
| 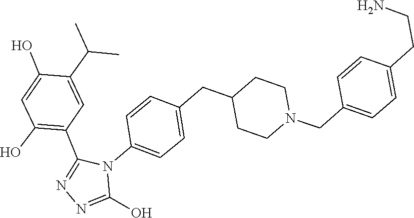 [SMILES: CC(C)c1cc(-c2nnc(O)n2-c2ccc(CC3CCN(Cc4ccc(CCN)cc4)CC3)cc2)c(O)cc1O] | 68 | 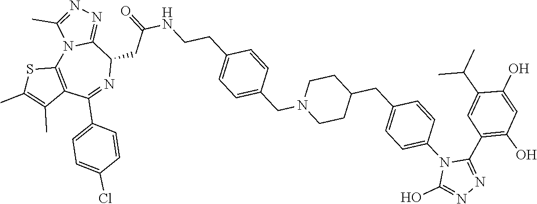 [SMILES: Cc1sc2c(c1C)C(c1ccc(Cl)cc1)=N[C@@H](CC(=O)NCCc1ccc(CN3CCC(Cc4ccc(-n5c(O)nnc5-c5cc(C(C)C)c(O)cc5O)cc4)CC3)cc1)c1nnc(C)n1-2] | 68 | 916.2 [M − CF3COOH + H]+ |
| 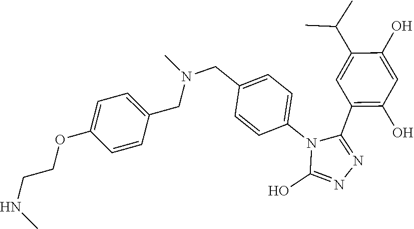 [SMILES: CNCCOc1ccc(CN(C)Cc2ccc(-n3c(O)nnc3-c3cc(C(C)C)c(O)cc3O)cc2)cc1] | 69 | 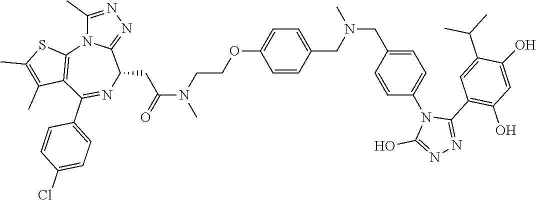 [SMILES: Cc1sc2c(c1C)C(c1ccc(Cl)cc1)=N[C@@H](CC(=O)N(C)CCOc1ccc(CN(C)Cc3ccc(-n4c(O)nnc4-c4cc(C(C)C)c(O)cc4O)cc3)cc1)c1nnc(C)n1-2] | 69 | 1015.2 [M − CF3COOH + H]+ |
| 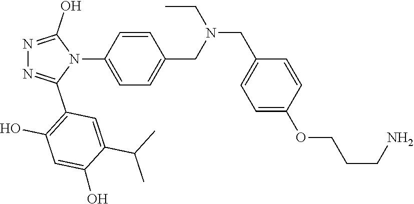 [SMILES: CCN(Cc1ccc(OCCCN)cc1)Cc1ccc(-n2c(O)nnc2-c2cc(C(C)C)c(O)cc2O)cc1] | 70 | 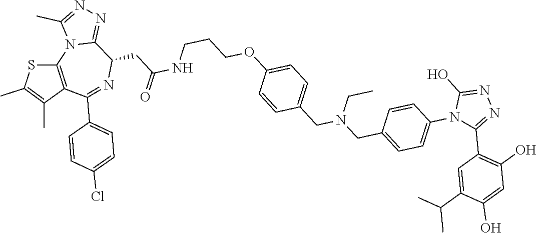 [SMILES: CCN(Cc1ccc(OCCCNC(=O)C[C@@H]2N=C(c3ccc(Cl)cc3)c3c(sc(C)c3C)-n3c(C)nnc32)cc1)Cc1ccc(-n2c(O)nnc2-c2cc(C(C)C)c(O)cc2O)cc1] | 70 | 1013.2 [M − CF3COOH + H]+ |
| 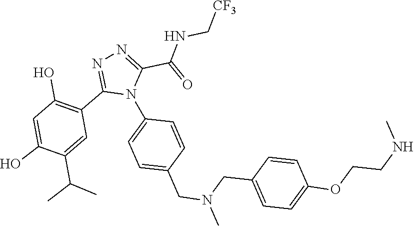 [SMILES: CNCCOc1ccc(CN(C)Cc2ccc(-n3c(C(=O)NCC(F)(F)F)nnc3-c3cc(C(C)C)c(O)cc3O)cc2)cc1] | 71 | 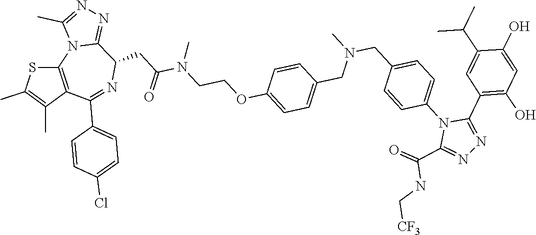 [SMILES: Cc1sc2c(c1C)C(c1ccc(Cl)cc1)=N[C@@H](CC(=O)N(C)CCOc1ccc(CN(C)Cc3ccc(-n4c(C(=O)NCC(F)(F)F)nnc4-c4cc(C(C)C)c(O)cc4O)cc3)cc1)c1nnc(C)n1-2] | 71 | 10094 [M − HCOOH + H]+ |
| 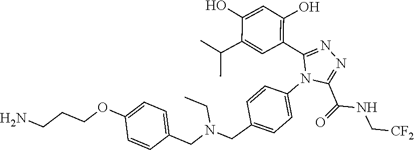 | 72 | 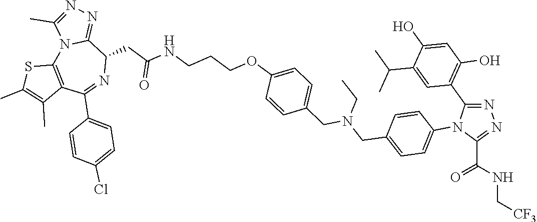 [SMILES: CCN(Cc1ccc(OCCCNC(=O)C[C@@H]2N=C(c3ccc(Cl)cc3)c3c(sc(C)c3C)-n3c(C)nnc32)cc1)Cc1ccc(-n2c(C(=O)NCC(F)(F)F)nnc2-c2cc(C(C)C)c(O)cc2O)cc1] | 72 | 1023.2 [M − CF3COOH + H]+ |
| 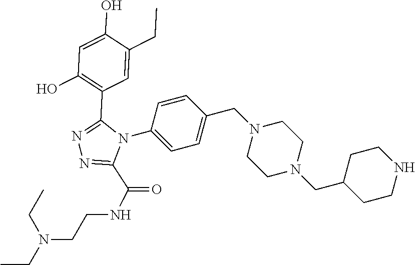 [SMILES: CCc1cc(-c2nnc(C(=O)NCCN(CC)CC)n2-c2ccc(CN3CCN(CC4CCNCC4)CC3)cc2)c(O)cc1O] | 73 | 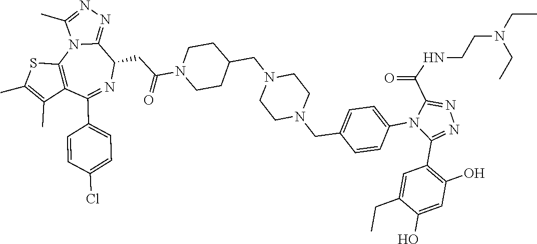 [SMILES: CCc1cc(-c2nnc(C(=O)NCCN(CC)CC)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 73 | 1001.5 [M − CF3COOH + H]+ |
| 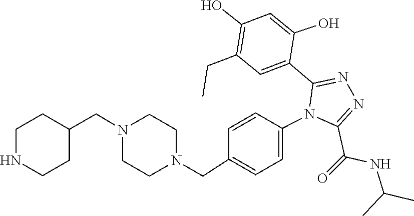 [SMILES: CCc1cc(-c2nnc(C(=O)NC(C)C)n2-c2ccc(CN3CCN(CC4CCNCC4)CC3)cc2)c(O)cc1O] | 74 | 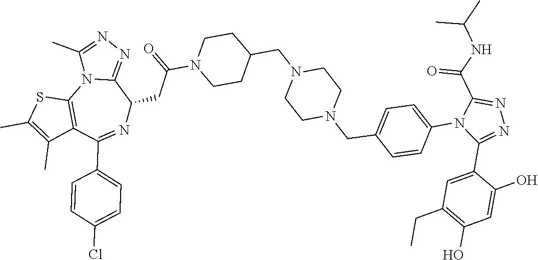 [SMILES: CCc1cc(-c2nnc(C(=O)NC(C)C)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 74 | 874.2 [MCF3COOH + H]+ |
| 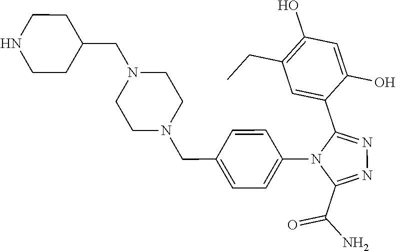 [SMILES: CCc1cc(-c2nnc(C(N)=O)n2-c2ccc(CN3CCN(CC4CCNCC4)CC3)cc2)c(O)cc1O] | 75 | 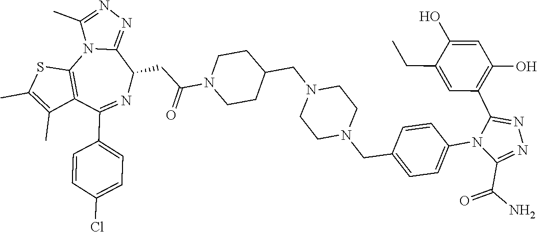 [SMILES: CCc1cc(-c2nnc(C(N)=O)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 75 | 973.3 [MCF3COOH + H]+ |
| 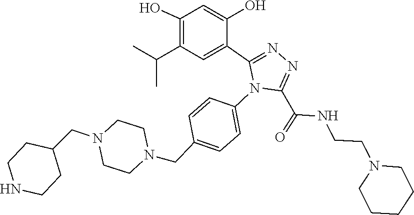 [SMILES: CC(C)c1cc(-c2nnc(C(=O)NCCN3CCCCC3)n2-c2ccc(CN3CCN(CC4CCNCC4)CC3)cc2)c(O)cc1O] | 76 | 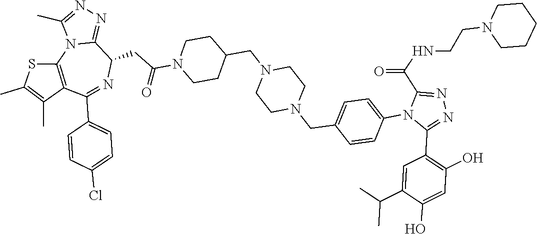 [SMILES: Cc1sc2c(c1C)C(c1ccc(Cl)cc1)=N[C@@H](CC(=O)N1CCC(CN3CCN(Cc4ccc(-n5c(C(=O)NCCN6CCCCC6)nnc5-c5cc(C(C)C)c(O)cc5O)cc4)CC3)CC1)c1nnc(C)n1-2] | 76 | 985.5 [M − CF3COOH + H]+ |
| 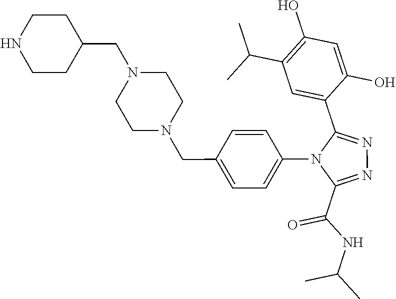 [SMILES: CC(C)NC(=O)c1nnc(-c2cc(C(C)C)c(O)cc2O)n1-c1ccc(CN2CCN(CC3CCNCC3)CC2)cc1] | 77 | 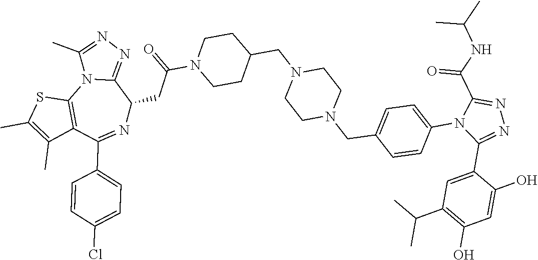 [SMILES: Cc1sc2c(c1C)C(c1ccc(Cl)cc1)=N[C@@H](CC(=O)N1CCC(CN3CCN(Cc4ccc(-n5c(C(=O)NC(C)C)nnc5-c5cc(C(C)C)c(O)cc5O)cc4)CC3)CC1)c1nnc(C)n1-2] | 77 | 916.2 [M − CF3COOH + H]+ |
| 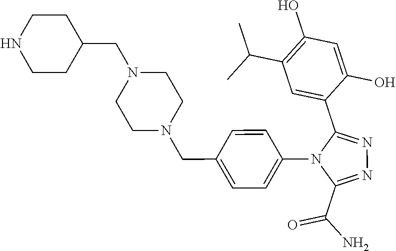 [SMILES: CC(C)c1cc(-c2nnc(C(N)=O)n2-c2ccc(CN3CCN(CC4CCNCC4)CC3)cc2)c(O)cc1O] | 78 | 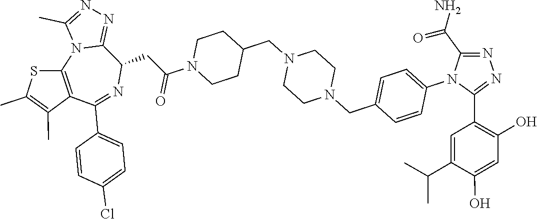 [SMILES: Cc1sc2c(c1C)C(c1ccc(Cl)cc1)=N[C@@H](CC(=O)N1CCC(CN3CCN(Cc4ccc(-n5c(C(N)=O)nnc5-c5cc(C(C)C)c(O)cc5O)cc4)CC3)CC1)c1nnc(C)n1-2] | 78 | 958.2 [MCF3COOH + H]+ |
| 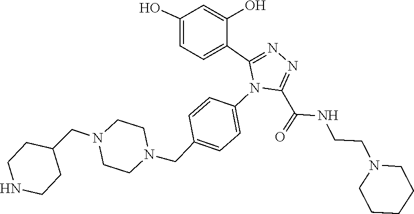 [SMILES: O=C(NCCN1CCCCC1)c1nnc(-c2ccc(O)cc2O)n1-c1ccc(CN2CCN(CC3CCNCC3)CC2)cc1] | 79 | 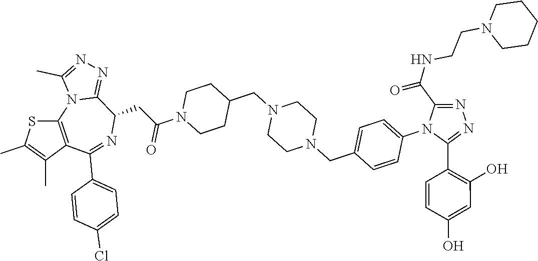 [SMILES: Cc1sc2c(c1C)C(c1ccc(Cl)cc1)=N[C@@H](CC(=O)N1CCC(CN3CCN(Cc4ccc(-n5c(C(=O)NCCN6CCCCC6)nnc5-c5ccc(O)cc5O)cc4)CC3)CC1)c1nnc(C)n1-2] | 79 | 1027.6 [M − CF3COOH + H]+ |
| 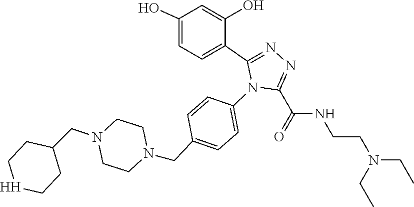 | 80 | 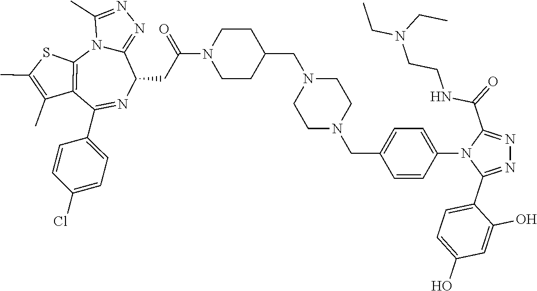 [SMILES: CCN(CC)CCNC(=O)c1nnc(-c2ccc(O)cc2O)n1-c1ccc(CN2CCN(CC3CCN(C(=O)C[C@@H]4N=C(c5ccc(Cl)cc5)c5c(sc(C)c5C)-n5c(C)nnc54)CC3)CC2)cc1] | 80 | 902.2 [M − CF3COOH + H]+ |
| 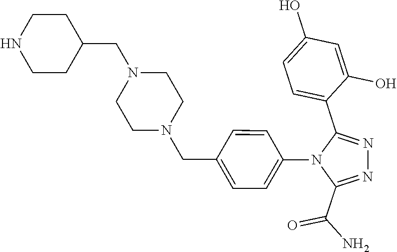 [SMILES: NC(=O)c1nnc(-c2ccc(O)cc2O)n1-c1ccc(CN2CCN(CC3CCNCC3)CC2)cc1] | 81 | 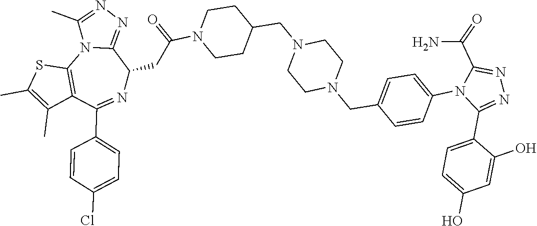 [SMILES: Cc1sc2c(c1C)C(c1ccc(Cl)cc1)=N[C@@H](CC(=O)N1CCC(CN3CCN(Cc4ccc(-n5c(C(N)=O)nnc5-c5ccc(O)cc5O)cc4)CC3)CC1)c1nnc(C)n1-2] | 81 | 944.2 [M − CF3COOH + H]+ |
| 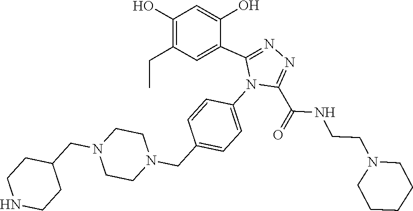 [SMILES: CCc1cc(-c2nnc(C(=O)NCCN3CCCCC3)n2-c2ccc(CN3CCN(CC4CCNCC4)CC3)cc2)c(O)cc1O] | 82 | 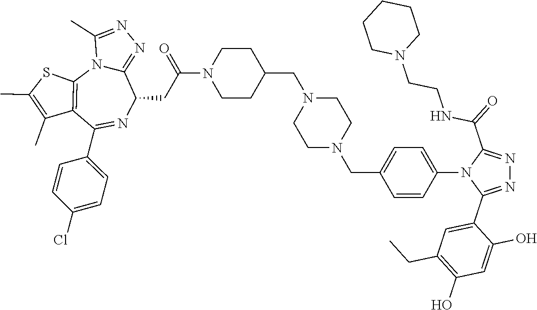 [SMILES: CCc1cc(-c2nnc(C(=O)NCCN3CCCCC3)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 82 | 914.2 [M − HCOOH + H]+ |
| 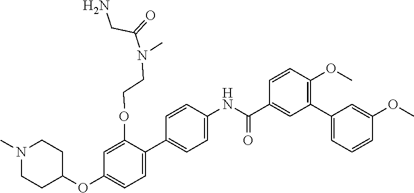 | 83 | 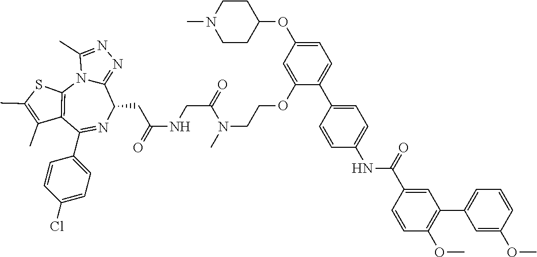 | 83 | 929.3 [M − CF3COOH + H]+ |
| 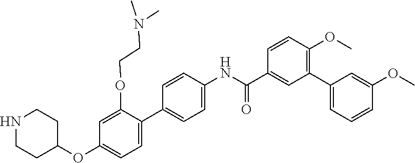 [SMILES: COc1cccc(-c2cc(C(=O)Nc3ccc(-c4ccc(OC5CCNCC5)cc4OCCN(C)C)cc3)ccc2OC)c1] | 84 | 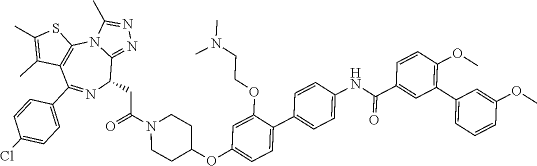 [SMILES: COc1cccc(-c2cc(C(=O)Nc3ccc(-c4ccc(OC5CCN(C(=O)C[C@@H]6N=C(c7ccc(Cl)cc7)c7c(sc(C)c7C)-n7c(C)nnc76)CC5)cc4OCCN(C)C)cc3)ccc2OC)c1] | 84 | 997.3 [M − CF3COOH + H]+ |
| 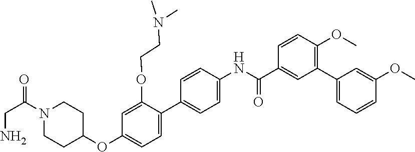 [SMILES: COc1cccc(-c2cc(C(=O)Nc3ccc(-c4ccc(OC5CCN(C(=O)CN)CC5)cc4OCCN(C)C)cc3)ccc2OC)c1] | 85 | 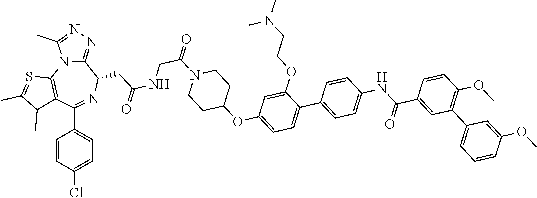 | 85 | 983.4 [M − CF3COOH + H]+ |
| 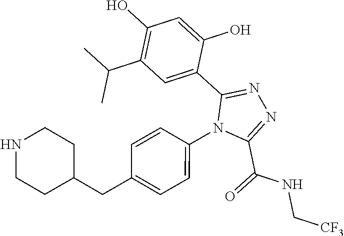 [SMILES: CC(C)c1cc(-c2nnc(C(=O)NCC(F)(F)F)n2-c2ccc(CC3CCNCC3)cc2)c(O)cc1O] | 86 | 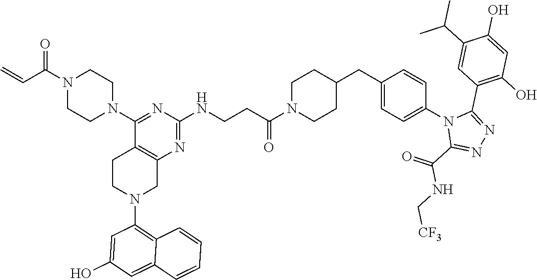 [SMILES: C=CC(=O)N1CCN(c2nc(NCCC(=O)N3CCC(Cc4ccc(-n5c(C(=O)NCC(F)(F)F)nnc5-c5cc(C(C)C)c(O)cc5O)cc4)CC3)nc3c2CCN(c2cc(O)cc4ccccc24)C3)CC1] | 86 | 1003.0 [M − CF3COOH + H]+ |
| 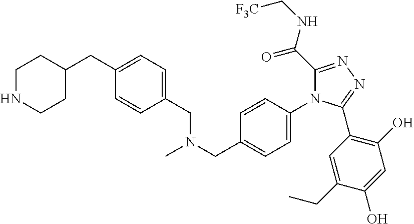 [SMILES: CCc1cc(-c2nnc(C(=O)NCC(F)(F)F)n2-c2ccc(CN(C)Cc3ccc(CC4CCNCC4)cc3)cc2)c(O)cc1O] | 87 | 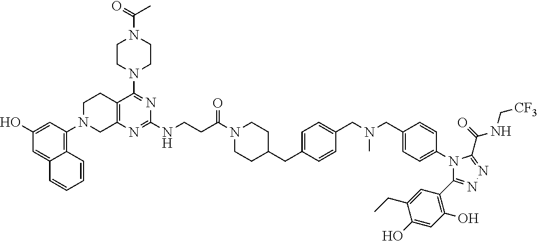 [SMILES: CCc1cc(-c2nnc(C(=O)NCC(F)(F)F)n2-c2ccc(CN(C)Cc3ccc(CC4CCN(C(=O)CCNc5nc6c(c(N7CCN(C(C)=O)CC7)n5)CCN(c5cc(O)cc7ccccc57)C6)CC4)cc3)cc2)c(O)cc1O] | 87 | 1109.3 [M − CF3COOH + H]+ |
| 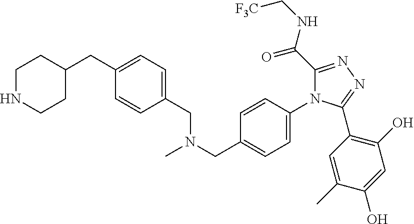 [SMILES: Cc1cc(-c2nnc(C(=O)NCC(F)(F)F)n2-c2ccc(CN(C)Cc3ccc(CC4CCNCC4)cc3)cc2)c(O)cc1O] | 88 | 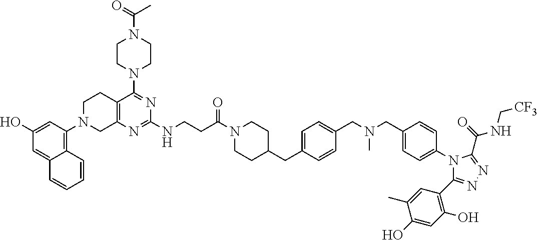 [SMILES: CC(=O)N1CCN(c2nc(NCCC(=O)N3CCC(Cc4ccc(CN(C)Cc5ccc(-n6c(C(=O)NCC(F)(F)F)nnc6-c6cc(C)c(O)cc6O)cc5)cc4)CC3)nc3c2CCN(c2cc(O)cc4ccccc24)C3)CC1] | 88 | 1095.3 [M − CF3COOH + H]+ |
| 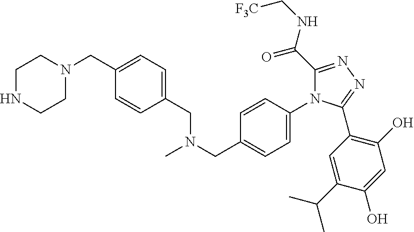 [SMILES: CC(C)c1cc(-c2nnc(C(=O)NCC(F)(F)F)n2-c2ccc(CN(C)Cc3ccc(CN4CCNCC4)cc3)cc2)c(O)cc1O] | 89 | 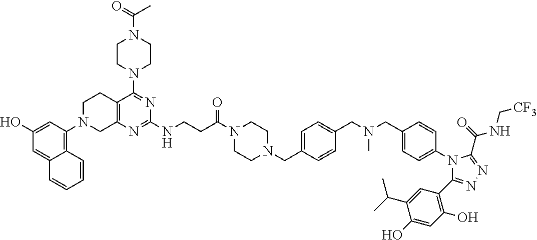 [SMILES: CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1] | 89 | 1124.3 [M − CF3COOH + H]+ |
| 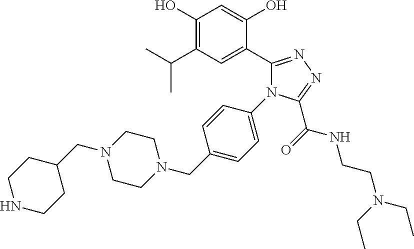 [SMILES: CCN(CC)CCNC(=O)c1nnc(-c2cc(C(C)C)c(O)cc2O)n1-c1ccc(CN2CCN(CC3CCNCC3)CC2)cc1] | 90 | 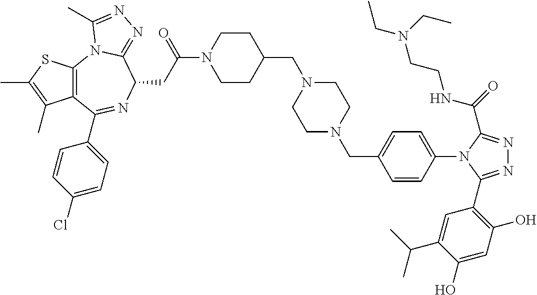 [SMILES: CCN(CC)CCNC(=O)c1nnc(-c2cc(C(C)C)c(O)cc2O)n1-c1ccc(CN2CCN(CC3CCN(C(=O)C[C@@H]4N=C(c5ccc(Cl)cc5)c5c(sc(C)c5C)-n5c(C)nnc54)CC3)CC2)cc1] | 90 | 900.2 [M − HCOOH + H]+ |
| 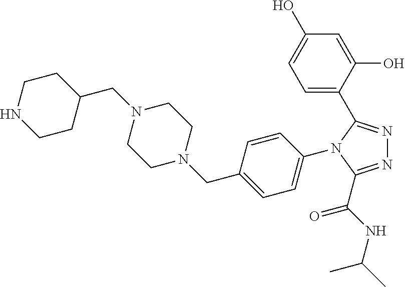 [SMILES: CC(C)NC(=O)c1nnc(-c2ccc(O)cc2O)n1-c1ccc(CN2CCN(CC3CCNCC3)CC2)cc1] | 91 | 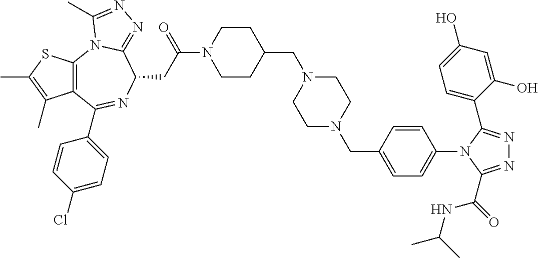 [SMILES: Cc1sc2c(c1C)C(c1ccc(Cl)cc1)=N[C@@H](CC(=O)N1CCC(CN3CCN(Cc4ccc(-n5c(C(=O)NC(C)C)nnc5-c5ccc(O)cc5O)cc4)CC3)CC1)c1nnc(C)n1-2] | 91 | 924.3 [M − CF3COOH + H]+ |
| 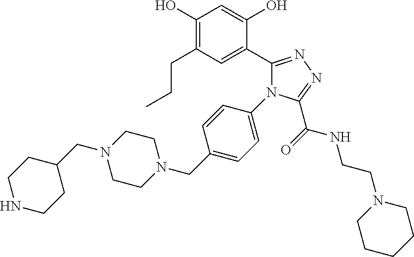 [SMILES: CCCc1cc(-c2nnc(C(=O)NCCN3CCCCC3)n2-c2ccc(CN3CCN(CC4CCNCC4)CC3)cc2)c(O)cc1O] | 92 | 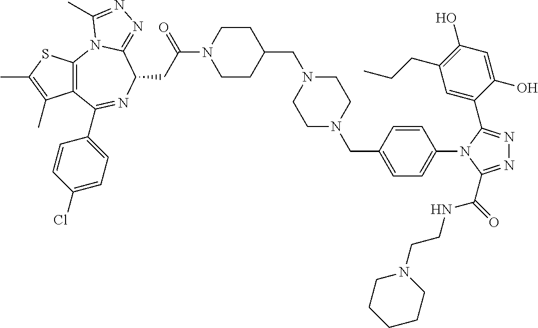 [SMILES: CCCc1cc(-c2nnc(C(=O)NCCN3CCCCC3)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 92 | 938.3 [M − CF3COOH + H]+ |
| 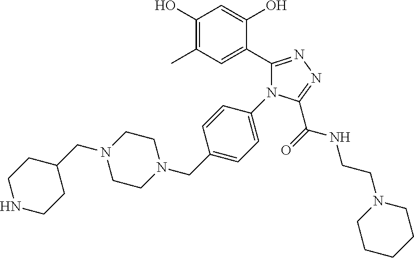 | 93 | 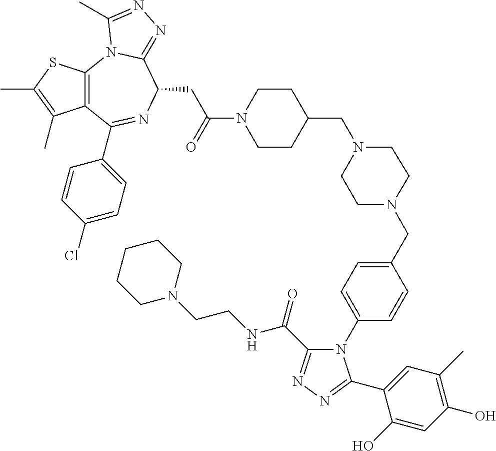 [SMILES: Cc1cc(-c2nnc(C(=O)NCCN3CCCCC3)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 93 | 943.0 [M + H]+ |
| 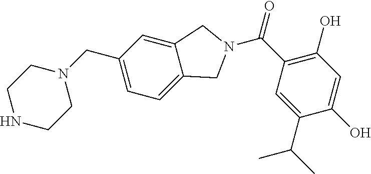 [SMILES: CC(C)c1cc(C(=O)N2Cc3ccc(CN4CCNCC4)cc3C2)c(O)cc1O] | 94 | 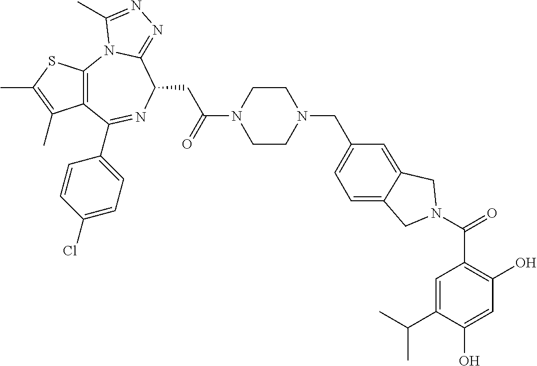 [SMILES: Cc1sc2c(c1C)C(c1ccc(Cl)cc1)=N[C@@H](CC(=O)N1CCN(Cc3ccc4c(c3)CN(C(=O)c3cc(C(C)C)c(O)cc3O)C4)CC1)c1nnc(C)n1-2] | 94 | 924.2 [M − CF3COOH + H]+ |
| 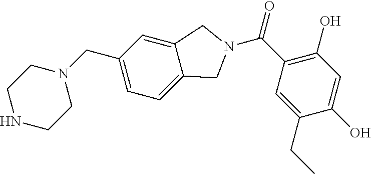 [SMILES: CCc1cc(C(=O)N2Cc3ccc(CN4CCNCC4)cc3C2)c(O)cc1O] | 95 | 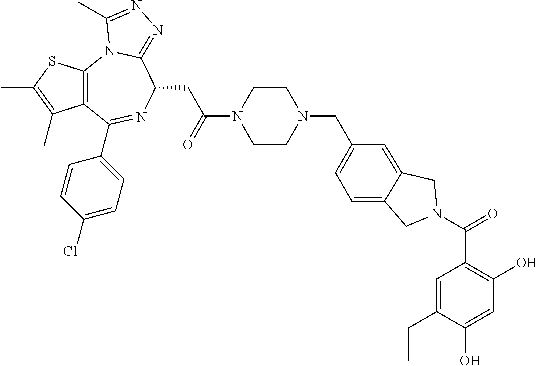 [SMILES: CCc1cc(C(=O)N2Cc3ccc(CN4CCN(C(=O)C[C@@H]5N=C(c6ccc(Cl)cc6)c6c(sc(C)c6C)-n6c(C)nnc65)CC4)cc3C2)c(O)cc1O] | 95 | 924.6 [M − HCl + H]+ |
| 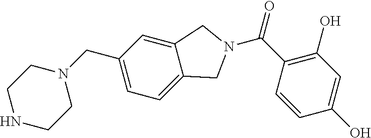 [SMILES: O=C(c1ccc(O)cc1O)N1Cc2ccc(CN3CCNCC3)cc2C1] | 96 | 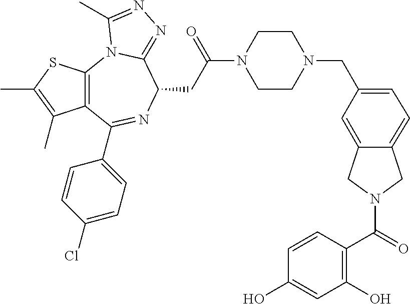 [SMILES: Cc1sc2c(c1C)C(c1ccc(Cl)cc1)=N[C@@H](CC(=O)N1CCN(Cc3ccc4c(c3)CN(C(=O)c3ccc(O)cc3O)C4)CC1)c1nnc(C)n1-2] | 96 | 924.3 [M − CF3COOH + H]+ |
| 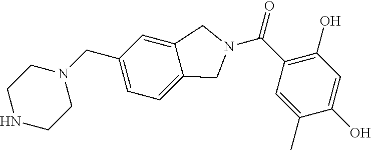 | 97 | 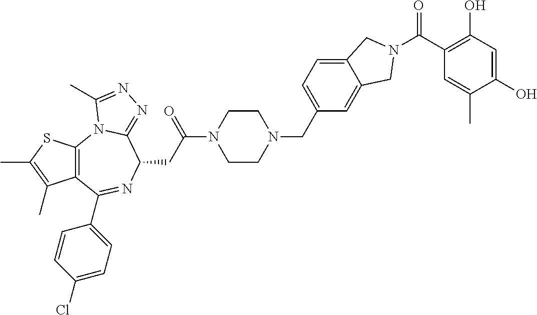 | 97 | 778.1 [M + H]+ |
| 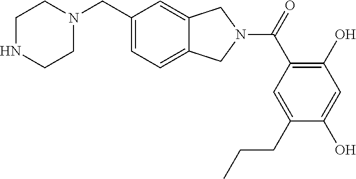 [SMILES: CCCc1cc(C(=O)N2Cc3ccc(CN4CCNCC4)cc3C2)c(O)cc1O] | 98 | 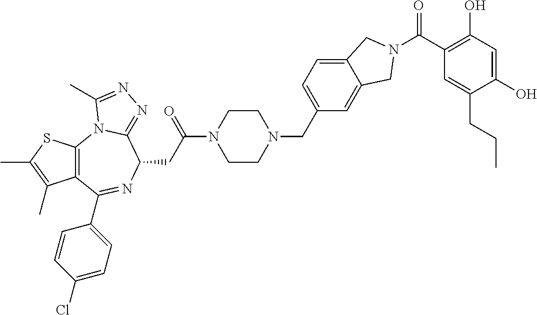 [SMILES: CCCc1cc(C(=O)N2Cc3ccc(CN4CCN(C(=O)C[C@@H]5N=C(c6ccc(Cl)cc6)c6c(sc(C)c6C)-n6c(C)nnc65)CC4)cc3C2)c(O)cc1O] | 98 | 984.4 [M − HCl + H]+ |
| 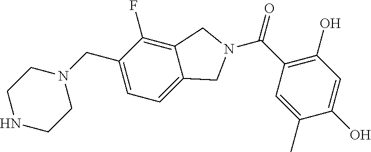 | 99 | 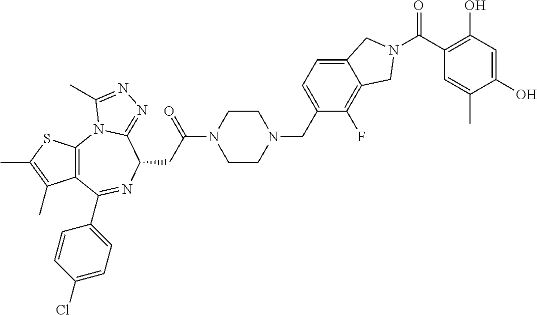 [SMILES: Cc1cc(C(=O)N2Cc3ccc(CN4CCN(C(=O)C[C@@H]5N=C(c6ccc(Cl)cc6)c6c(sc(C)c6C)-n6c(C)nnc65)CC4)c(F)c3C2)c(O)cc1O] | 99 | 768.7 [M + H]+ |
| 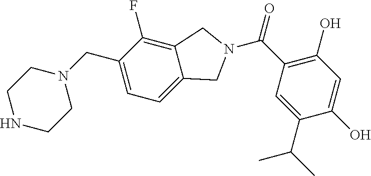 | 100 | 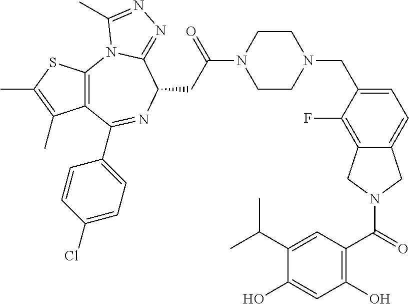 [SMILES: Cc1sc2c(c1C)C(c1ccc(Cl)cc1)=N[C@@H](CC(=O)N1CCN(Cc3ccc4c(c3F)CN(C(=O)c3cc(C(C)C)c(O)cc3O)C4)CC1)c1nnc(C)n1-2] | 100 | 796.2 [M + H]+ |
| 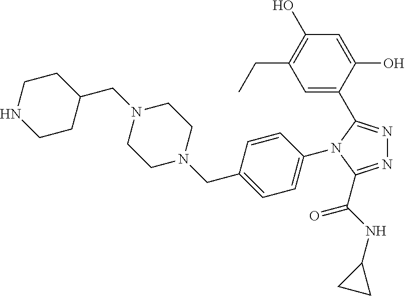 [SMILES: CCc1cc(-c2nnc(C(=O)NC3CC3)n2-c2ccc(CN3CCN(CC4CCNCC4)CC3)cc2)c(O)cc1O] | 101 | 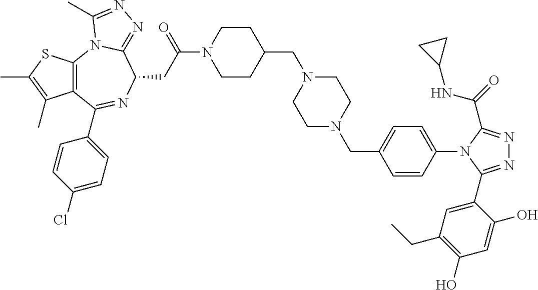 [SMILES: CCc1cc(-c2nnc(C(=O)NC3CC3)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 101 | 942.2 [M − CF3COOH + H]+ |
| 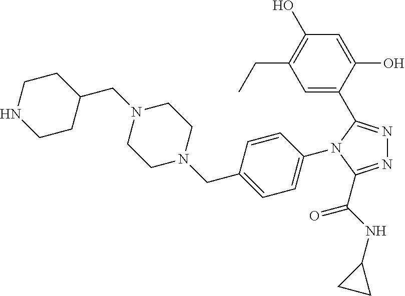 [SMILES: CCc1cc(-c2nnc(C(=O)NC3CC3)n2-c2ccc(CN3CCN(CC4CCNCC4)CC3)cc2)c(O)cc1O] | 102 | 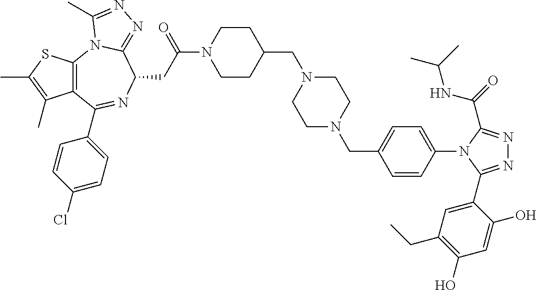 [SMILES: CCc1cc(-c2nnc(C(=O)NC(C)C)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 102 | 944.2 [M − CF3COOH + H]+ |
| 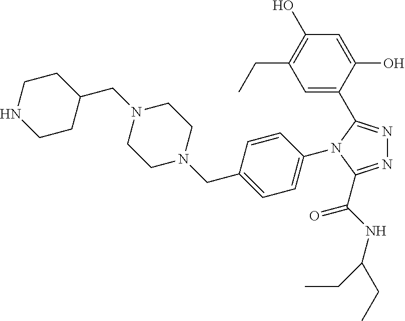 [SMILES: CCc1cc(-c2nnc(C(=O)NC(CC)CC)n2-c2ccc(CN3CCN(CC4CCNCC4)CC3)cc2)c(O)cc1O] | 103 | 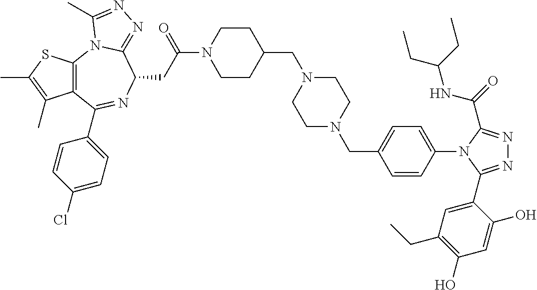 [SMILES: CCc1cc(-c2nnc(C(=O)NC(CC)CC)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 103 | 973.4 [M − CF3COOH + H]+ |
| 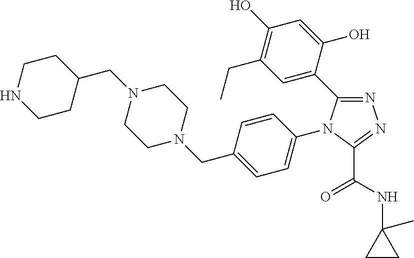 [SMILES: CCc1cc(-c2nnc(C(=O)NC3(C)CC3)n2-c2ccc(CN3CCN(CC4CCNCC4)CC3)cc2)c(O)cc1O] | 104 | 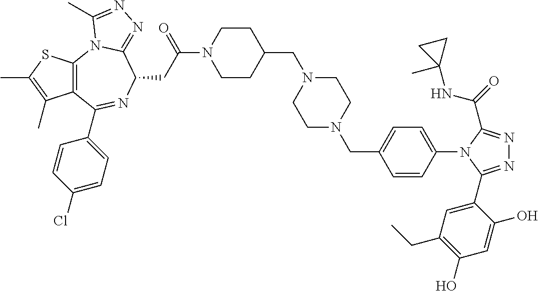 [SMILES: CCc1cc(-c2nnc(C(=O)NC3(C)CC3)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 104 | 956.6 [M − CF3COOH + H]+ |
| 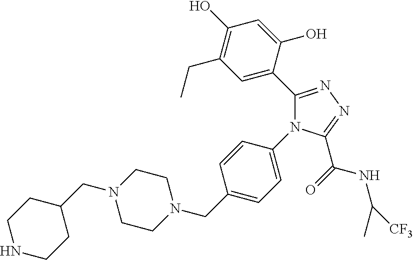 [SMILES: CCc1cc(-c2nnc(C(=O)NC(C)C(F)(F)F)n2-c2ccc(CN3CCN(CC4CCNCC4)CC3)cc2)c(O)cc1O] | 105 | 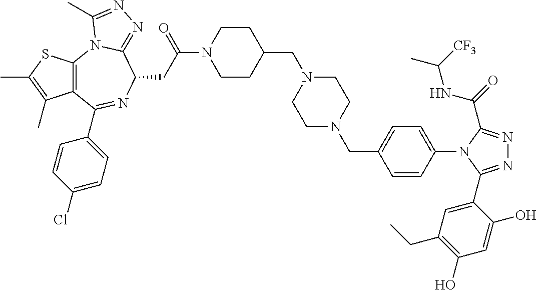 [SMILES: CCc1cc(-c2nnc(C(=O)NC(C)C(F)(F)F)n2-c2ccc(CN3CCN(CC4CCN(C(=O)C[C@@H]5N=C(c6ccc(Cl)cc6)c6c(sc(C)c6C)-n6c(C)nnc65)CC4)CC3)cc2)c(O)cc1O] | 105 | 998.0 [M − CF3COOH + H]+ |
| 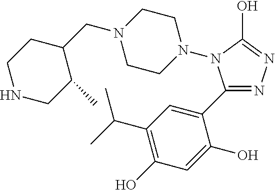 [SMILES: CC(C)c1cc(-c2nnc(O)n2N2CCN(CC3CCNC[C@H]3C)CC2)c(O)cc1O] | 106 | 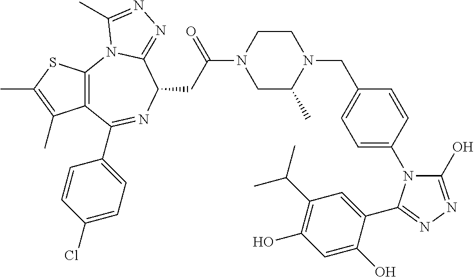 [SMILES: Cc1sc2c(c1C)C(c1ccc(Cl)cc1)=N[C@@H](CC(=O)N1CCN(Cc3ccc(-n4c(O)nnc4-c4cc(C(C)C)c(O)cc4O)cc3)[C@H](C)C1)c1nnc(C)n1-2] | 106 | 806.2 [M − CF3COOH + H]+ |
| 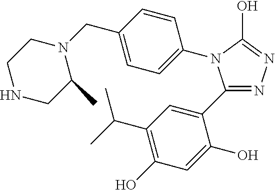 [SMILES: CC(C)c1cc(-c2nnc(O)n2-c2ccc(CN3CCNC[C@@H]3C)cc2)c(O)cc1O] | 107 | 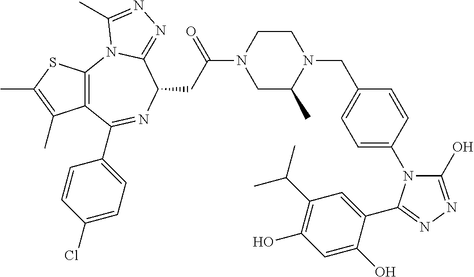 [SMILES: Cc1sc2c(c1C)C(c1ccc(Cl)cc1)=N[C@@H](CC(=O)N1CCN(Cc3ccc(-n4c(O)nnc4-c4cc(C(C)C)c(O)cc4O)cc3)[C@@H](C)C1)c1nnc(C)n1-2] | 107 | 806.2 [M − CF3COOH + H]+ |
| 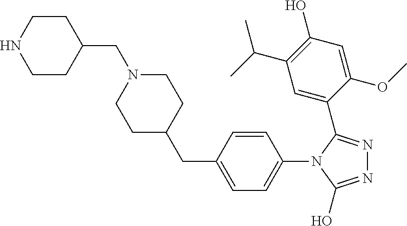 [SMILES: COc1cc(O)c(C(C)C)cc1-c1nnc(O)n1-c1ccc(CC2CCN(CC3CCNCC3)CC2)cc1] | 108 | 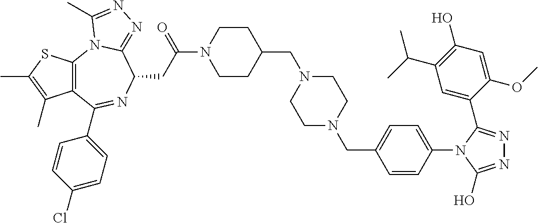 | 108 | 903.3 [M − CF3COOH + H]+ |
| 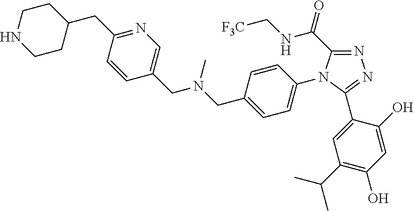 [SMILES: CC(C)c1cc(-c2nnc(C(=O)NCC(F)(F)F)n2-c2ccc(CN(C)Cc3ccc(CC4CCNCC4)nc3)cc2)c(O)cc1O] | 109 | 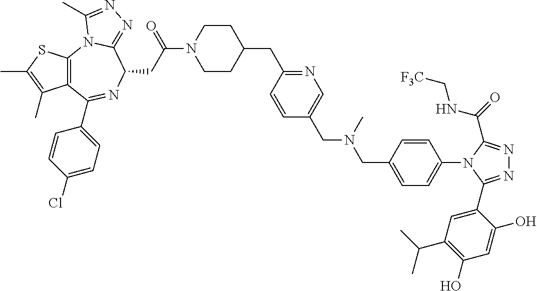 [SMILES: Cc1sc2c(c1C)C(c1ccc(Cl)cc1)=N[C@@H](CC(=O)N1CCC(Cc3ccc(CN(C)Cc4ccc(-n5c(C(=O)NCC(F)(F)F)nnc5-c5cc(C(C)C)c(O)cc5O)cc4)cn3)CC1)c1nnc(C)n1-2] | 109 | 1034.2 [M − CF3COOH + H]+ |
| 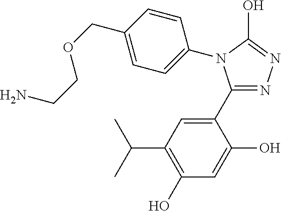 [SMILES: CC(C)c1cc(-c2nnc(O)n2-c2ccc(COCCN)cc2)c(O)cc1O] | 110 | 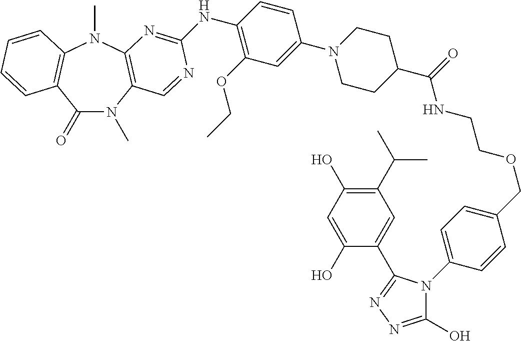 [SMILES: CCOc1cc(N2CCC(C(=O)NCCOCc3ccc(-n4c(O)nnc4-c4cc(C(C)C)c(O)cc4O)cc3)CC2)ccc1Nc1ncc2c(n1)N(C)c1ccccc1C(=O)N2C] | 110 | 869.5 [M − CF3COOH + H]+ |
| 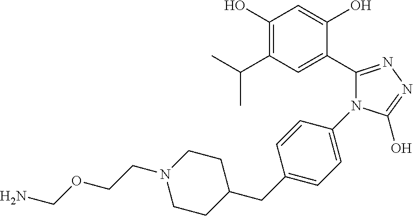 [SMILES: CC(C)c1cc(-c2nnc(O)n2-c2ccc(CC3CCN(CCOCN)CC3)cc2)c(O)cc1O] | 111 | 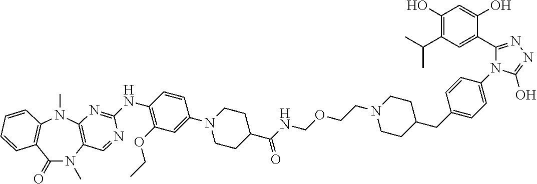 [SMILES: CCOc1cc(N2CCC(C(=O)NCOCCN3CCC(Cc4ccc(-n5c(O)nnc5-c5cc(C(C)C)c(O)cc5O)cc4)CC3)CC2)ccc1Nc1ncc2c(n1)N(C)c1ccccc1C(=O)N2C] | 111 | 981 [M − CF3COOH + H]+ |
| 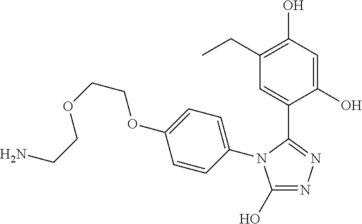 [SMILES: CCc1cc(-c2nnc(O)n2-c2ccc(OCCOCCN)cc2)c(O)cc1O] | 112 | 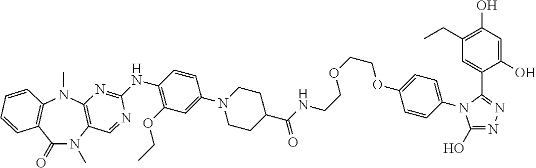 [SMILES: CCOc1cc(N2CCC(C(=O)NCCOCCOc3ccc(-n4c(O)nnc4-c4cc(CC)c(O)cc4O)cc3)CC2)ccc1Nc1ncc2c(n1)N(C)c1ccccc1C(=O)N2C] | 112 | 885.2 [M − CF3COOH + H]+ |
| 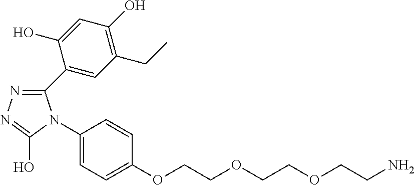 [SMILES: CCc1cc(-c2nnc(O)n2-c2ccc(OCCOCCOCCN)cc2)c(O)cc1O] | 113 | 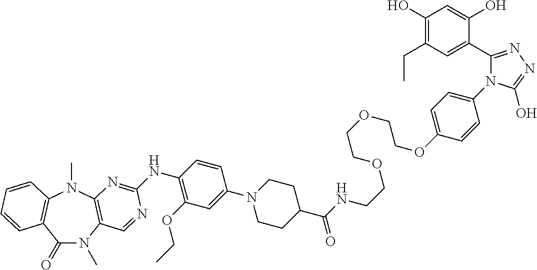 [SMILES: CCOc1cc(N2CCC(C(=O)NCCOCCOCCOc3ccc(-n4c(O)nnc4-c4cc(CC)c(O)cc4O)cc3)CC2)ccc1Nc1ncc2c(n1)N(C)c1ccccc1C(=O)N2C] | 113 | 929.2 [M − CF3COOH + H]+ |
| 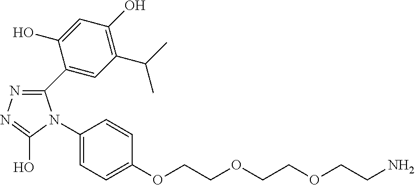 [SMILES: CC(C)c1cc(-c2nnc(O)n2-c2ccc(OCCOCCOCCN)cc2)c(O)cc1O] | 114 | 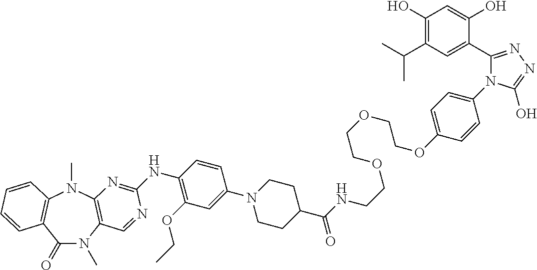 [SMILES: CCOc1cc(N2CCC(C(=O)NCCOCCOCCOc3ccc(-n4c(O)nnc4-c4cc(C(C)C)c(O)cc4O)cc3)CC2)ccc1Nc1ncc2c(n1)N(C)c1ccccc1C(=O)N2C] | 114 | 943.2 [M − CF3COOH + H]+ |
| 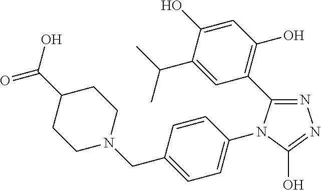 | 115 | 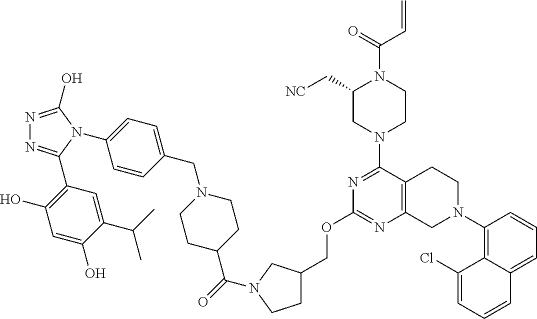 [SMILES: C=CC(=O)N1CCN(c2nc(OCC3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N] | 115 | 1006.4 [M + H]+ |
| 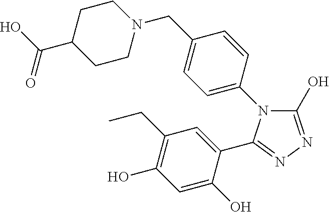 [SMILES: CCc1cc(-c2nnc(O)n2-c2ccc(CN3CCC(C(=O)O)CC3)cc2)c(O)cc1O] | 116 | 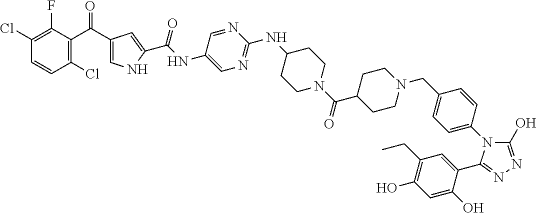 [SMILES: CCc1cc(-c2nnc(O)n2-c2ccc(CN3CCC(C(=O)N4CCC(Nc5ncc(NC(=O)c6cc(C(=O)c7c(Cl)ccc(Cl)c7F)c[nH]6)cn5)CC4)CC3)cc2)c(O)cc1O] | 116 | 897.5 [M − CF3COOH + H]+ |
| 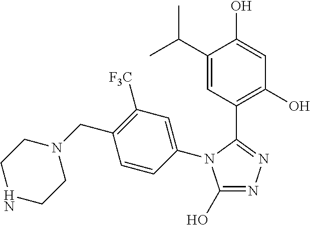 [SMILES: CC(C)c1cc(-c2nnc(O)n2-c2ccc(CN3CCNCC3)c(C(F)(F)F)c2)c(O)cc1O] | 117 | 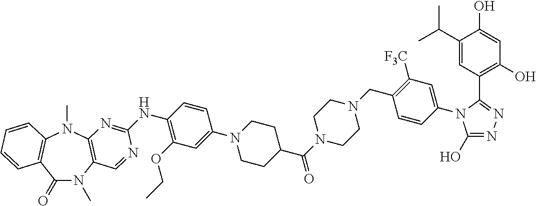 [SMILES: CCOc1cc(N2CCC(C(=O)N3CCN(Cc4ccc(-n5c(O)nnc5-c5cc(C(C)C)c(O)cc5O)cc4C(F)(F)F)CC3)CC2)ccc1Nc1ncc2c(n1)N(C)c1ccccc1C(=O)N2C] | 117 | 962.3 [M − CF3COOH + H]+ |
| 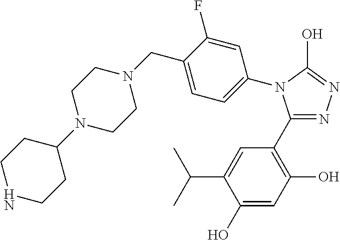 [SMILES: CC(C)c1cc(-c2nnc(O)n2-c2ccc(CN3CCN(C4CCNCC4)CC3)c(F)c2)c(O)cc1O] | 118 | 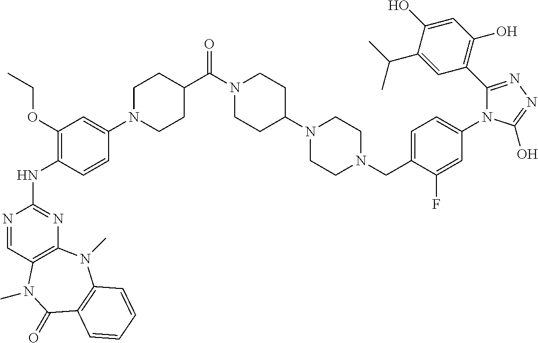 [SMILES: CCOc1cc(N2CCC(C(=O)N3CCC(N4CCN(Cc5ccc(-n6c(O)nnc6-c6cc(C(C)C)c(O)cc6O)cc5F)CC4)CC3)CC2)ccc1Nc1ncc2c(n1)N(C)c1ccccc1C(=O)N2C] | 118 | 996 [M − CF3COOH + H]+ |
| 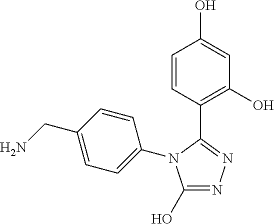 [SMILES: NCc1ccc(-n2c(O)nnc2-c2ccc(O)cc2O)cc1] | 119 | 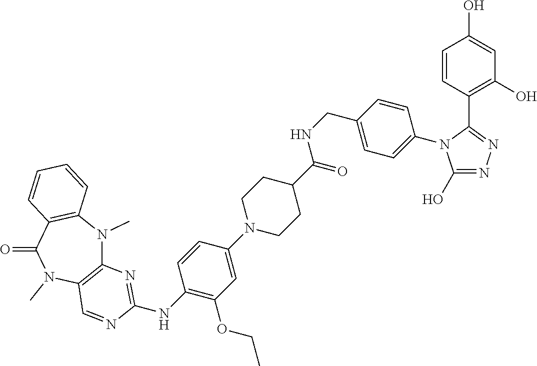 [SMILES: CCOc1cc(N2CCC(C(=O)NCc3ccc(-n4c(O)nnc4-c4ccc(O)cc4O)cc3)CC2)ccc1Nc1ncc2c(n1)N(C)c1ccccc1C(=O)N2C] | 119 | 783.3 [M − CF3COOH + H]+ |
| 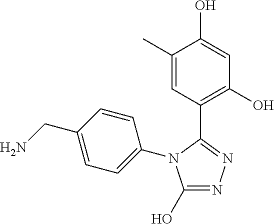 [SMILES: Cc1cc(-c2nnc(O)n2-c2ccc(CN)cc2)c(O)cc1O] | 120 | 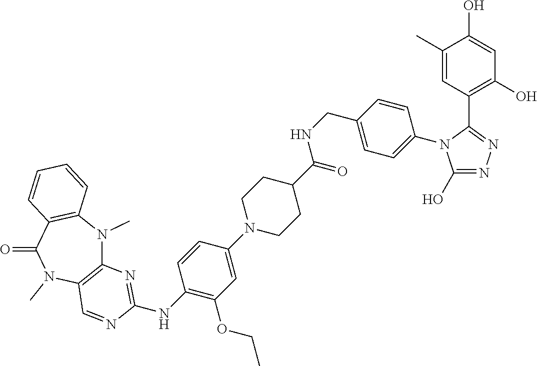 [SMILES: CCOc1cc(N2CCC(C(=O)NCc3ccc(-n4c(O)nnc4-c4cc(C)c(O)cc4O)cc3)CC2)ccc1Nc1ncc2c(n1)N(C)c1ccccc1C(=O)N2C] | 120 | 797.4 [M − CF3COOH + H]+ |
| 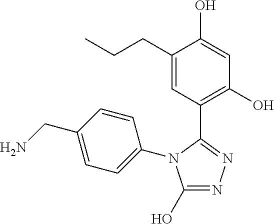 [SMILES: CCCc1cc(-c2nnc(O)n2-c2ccc(CN)cc2)c(O)cc1O] | 121 | 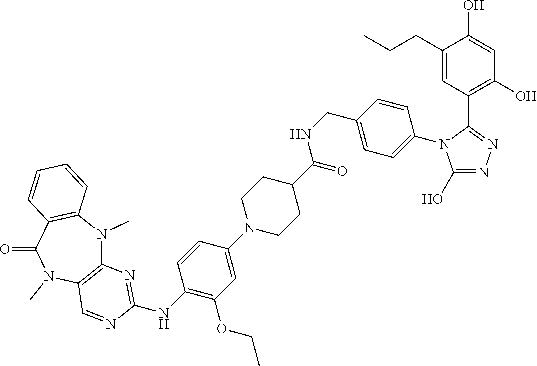 [SMILES: CCCc1cc(-c2nnc(O)n2-c2ccc(CNC(=O)C3CCN(c4ccc(Nc5ncc6c(n5)N(C)c5ccccc5C(=O)N6C)c(OCC)c4)CC3)cc2)c(O)cc1O] | 121 | 825.3 [M − CF3COOH + H]+ |
| 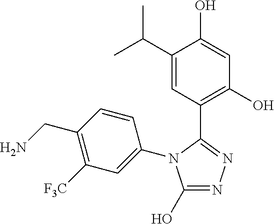 [SMILES: CC(C)c1cc(-c2nnc(O)n2-c2ccc(CN)c(C(F)(F)F)c2)c(O)cc1O] | 122 | 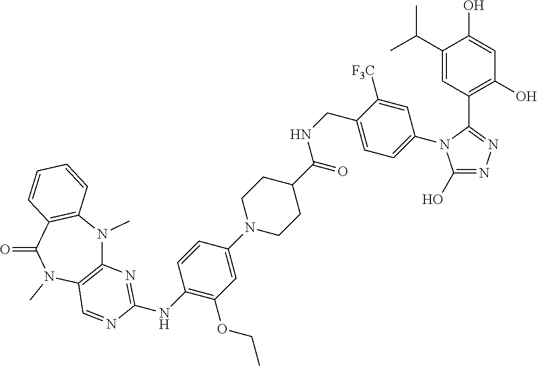 [SMILES: CCOc1cc(N2CCC(C(=O)NCc3ccc(-n4c(O)nnc4-c4cc(C(C)C)c(O)cc4O)cc3C(F)(F)F)CC2)ccc1Nc1ncc2c(n1)N(C)c1ccccc1C(=O)N2C] | 122 | 893.7 [M − CF3COOH + H]+ |
| 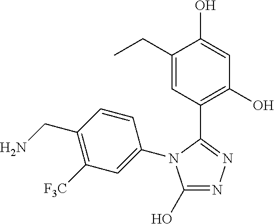 [SMILES: CCc1cc(-c2nnc(O)n2-c2ccc(CN)c(C(F)(F)F)c2)c(O)cc1O] | 123 | 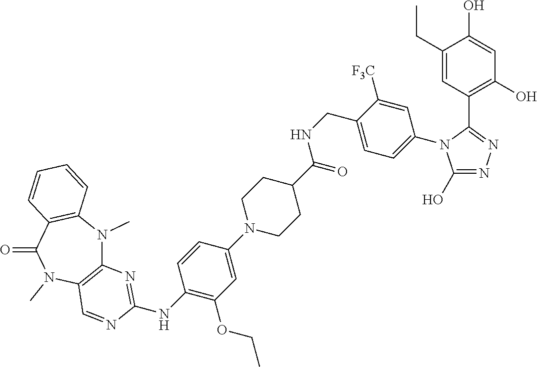 [SMILES: CCOc1cc(N2CCC(C(=O)NCc3ccc(-n4c(O)nnc4-c4cc(CC)c(O)cc4O)cc3C(F)(F)F)CC2)ccc1Nc1ncc2c(n1)N(C)c1ccccc1C(=O)N2C] | 123 | 879.7 [M − CF3COOH + H]+ |
| 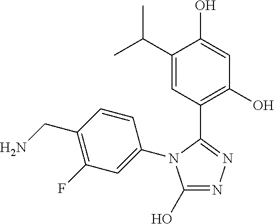 [SMILES: CC(C)c1cc(-c2nnc(O)n2-c2ccc(CN)c(F)c2)c(O)cc1O] | 124 | 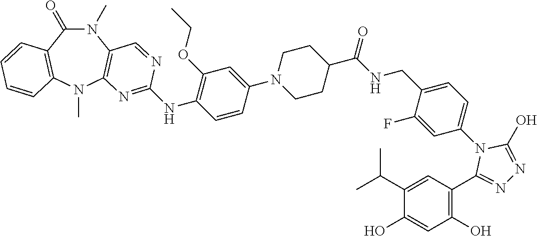 [SMILES: CCOc1cc(N2CCC(C(=O)NCc3ccc(-n4c(O)nnc4-c4cc(C(C)C)c(O)cc4O)cc3F)CC2)ccc1Nc1ncc2c(n1)N(C)c1ccccc1C(=O)N2C] | 124 | 843.8 [M − CF3COOH + H]+ |
| 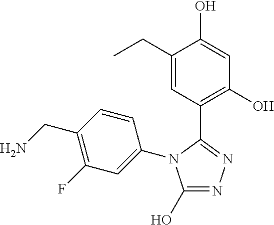 [SMILES: CCc1cc(-c2nnc(O)n2-c2ccc(CN)c(F)c2)c(O)cc1O] | 125 | 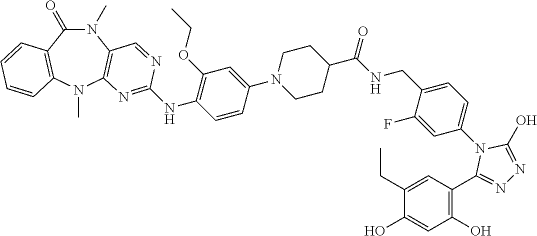 [SMILES: CCOc1cc(N2CCC(C(=O)NCc3ccc(-n4c(O)nnc4-c4cc(CC)c(O)cc4O)cc3F)CC2)ccc1Nc1ncc2c(n1)N(C)c1ccccc1C(=O)N2C] | 125 | 829.8 [M − CF3COOH + H]+ |
| 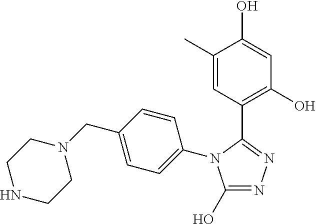 [SMILES: Cc1cc(-c2nnc(O)n2-c2ccc(CN3CCNCC3)cc2)c(O)cc1O] | 126 | 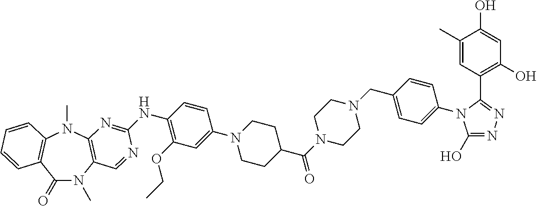 [SMILES: CCOc1cc(N2CCC(C(=O)N3CCN(Cc4ccc(-n5c(O)nnc5-c5cc(C)c(O)cc5O)cc4)CC3)CC2)ccc1Nc1ncc2c(n1)N(C)c1ccccc1C(=O)N2C] | 126 | 866.3 [M − CF3COOH + H]+ |
| 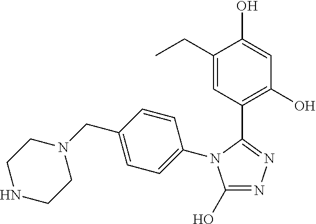 [SMILES: CCc1cc(-c2nnc(O)n2-c2ccc(CN3CCNCC3)cc2)c(O)cc1O] | 127 | 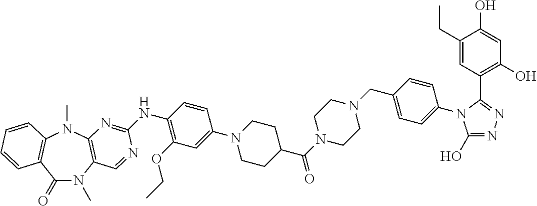 [SMILES: CCOc1cc(N2CCC(C(=O)N3CCN(Cc4ccc(-n5c(O)nnc5-c5cc(CC)c(O)cc5O)cc4)CC3)CC2)ccc1Nc1ncc2c(n1)N(C)c1ccccc1C(=O)N2C] | 127 | 880.3 [M − CF3COOH + H]+ |
| 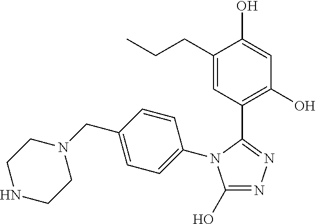 [SMILES: CCCc1cc(-c2nnc(O)n2-c2ccc(CN3CCNCC3)cc2)c(O)cc1O] | 128 | 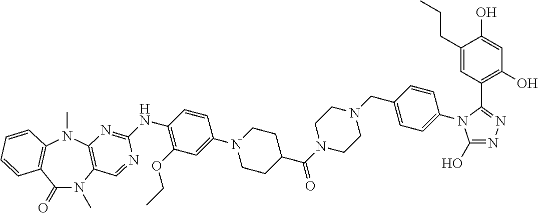 [SMILES: CCCc1cc(-c2nnc(O)n2-c2ccc(CN3CCN(C(=O)C4CCN(c5ccc(Nc6ncc7c(n6)N(C)c6ccccc6C(=O)N7C)c(OCC)c5)CC4)CC3)cc2)c(O)cc1O] | 128 | 894.9 [M − CF3COOH + H]+ |
| 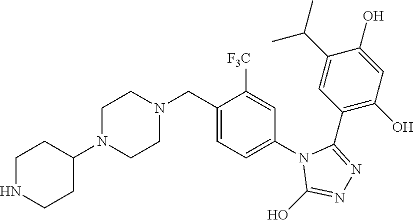 [SMILES: CC(C)c1cc(-c2nnc(O)n2-c2ccc(CN3CCN(C4CCNCC4)CC3)c(C(F)(F)F)c2)c(O)cc1O] | 129 | 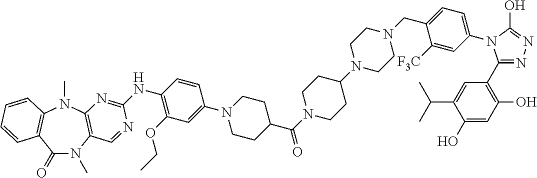 [SMILES: CCOc1cc(N2CCC(C(=O)N3CCC(N4CCN(Cc5ccc(-n6c(O)nnc6-c6cc(C(C)C)c(O)cc6O)cc5C(F)(F)F)CC4)CC3)CC2)ccc1Nc1ncc2c(n1)N(C)c1ccccc1C(=O)N2C] | 129 | 1046 [M − CF3COOH + H]+ |
| 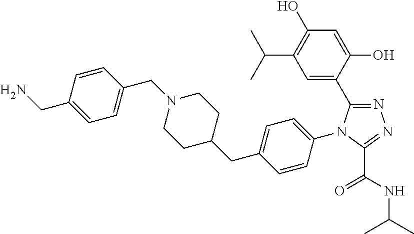 [SMILES: CC(C)NC(=O)c1nnc(-c2cc(C(C)C)c(O)cc2O)n1-c1ccc(CC2CCN(Cc3ccc(CN)cc3)CC2)cc1] | 130 | 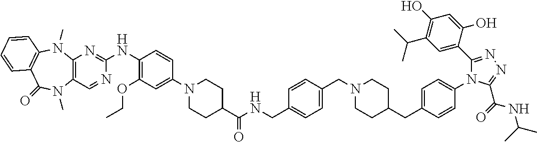 [SMILES: CCOc1cc(N2CCC(C(=O)NCc3ccc(CN4CCC(Cc5ccc(-n6c(C(=O)NC(C)C)nnc6-c6cc(C(C)C)c(O)cc6O)cc5)CC4)cc3)CC2)ccc1Nc1ncc2c(n1)N(C)c1ccccc1C(=O)N2C] | 130 | 1082.2 [M − CF3COOH + H]+ |
| 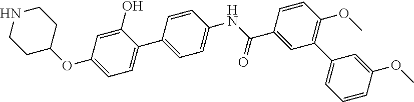 [SMILES: COc1cccc(-c2cc(C(=O)Nc3ccc(-c4ccc(OC5CCNCC5)cc4O)cc3)ccc2OC)c1] | 131 | 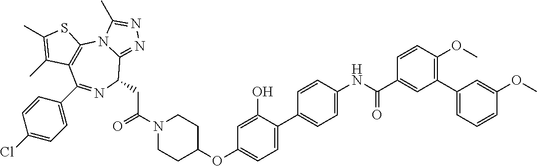 [SMILES: COc1cccc(-c2cc(C(=O)Nc3ccc(-c4ccc(OC5CCN(C(=O)C[C@@H]6N=C(c7ccc(Cl)cc7)c7c(sc(C)c7C)-n7c(C)nnc76)CC5)cc4O)cc3)ccc2OC)c1] | 131 | 939.3 [M − CF3COOH + H]+ |
| 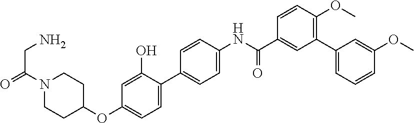 [SMILES: COc1cccc(-c2cc(C(=O)Nc3ccc(-c4ccc(OC5CCN(C(=O)CN)CC5)cc4O)cc3)ccc2OC)c1] | 132 | 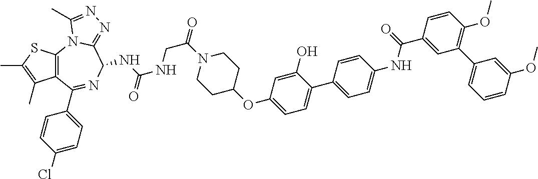 | 132 | 1005.2 [MCF3COOH + H]+ |
| 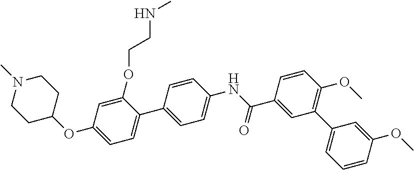 | 133 | 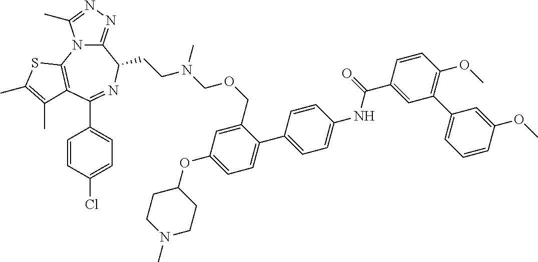 | 133 | 910.2 [MCF3COOH + H]+ |
| 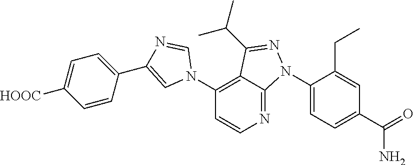 [SMILES: CCc1cc(C(N)=O)ccc1-n1nc(C(C)C)c2c(-n3cnc(-c4ccc(C(=O)O)cc4)c3)ccnc21] | 134 | | 134 | |
| 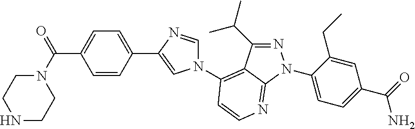 [SMILES: CCc1cc(C(N)=O)ccc1-n1nc(C(C)C)c2c(-n3cnc(-c4ccc(C(=O)N5CCNCC5)cc4)c3)ccnc21] | 135 | | 135 | |
| 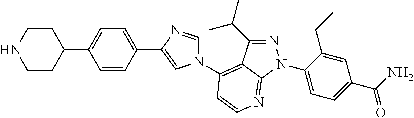 [SMILES: CCc1cc(C(N)=O)ccc1-n1nc(C(C)C)c2c(-n3cnc(-c4ccc(C5CCNCC5)cc4)c3)ccnc21] | 136 | | 136 | |
| 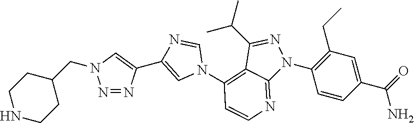 [SMILES: CCc1cc(C(N)=O)ccc1-n1nc(C(C)C)c2c(-n3cnc(-c4cn(CC5CCNCC5)nn4)c3)ccnc21] | 137 | | 137 | |
| 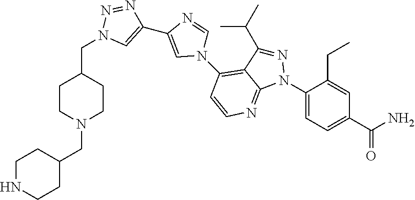 [SMILES: CCc1cc(C(N)=O)ccc1-n1nc(C(C)C)c2c(-n3cnc(-c4cn(CC5CCN(CC6CCNCC6)CC5)nn4)c3)ccnc21] | 138 | | 138 | |
| 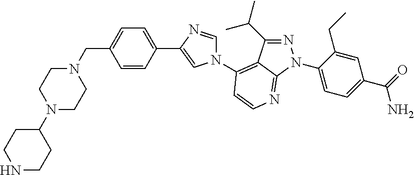 [SMILES: CCc1cc(C(N)=O)ccc1-n1nc(C(C)C)c2c(-n3cnc(-c4ccc(CN5CCN(C6CCNCC6)CC5)cc4)c3)ccnc21] | 139 | | 139 | |
| 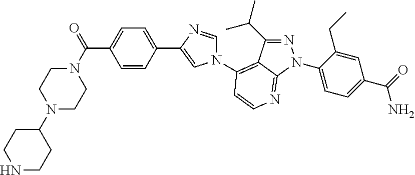 [SMILES: CCc1cc(C(N)=O)ccc1-n1nc(C(C)C)c2c(-n3cnc(-c4ccc(C(=O)N5CCN(C6CCNCC6)CC5)cc4)c3)ccnc21] | 140 | | 140 | |
| 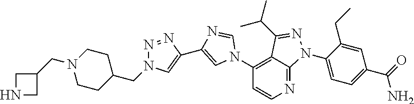 [SMILES: CCc1cc(C(N)=O)ccc1-n1nc(C(C)C)c2c(-n3cnc(-c4cn(CC5CCN(CC6CNC6)CC5)nn4)c3)ccnc21] | 141 | | 141 | |
| 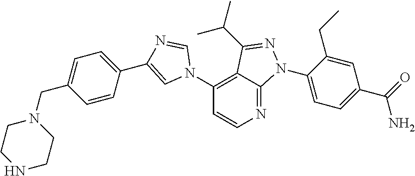 [SMILES: CCc1cc(C(N)=O)ccc1-n1nc(C(C)C)c2c(-n3cnc(-c4ccc(CN5CCNCC5)cc4)c3)ccnc21] | 142 | | 142 | |
| 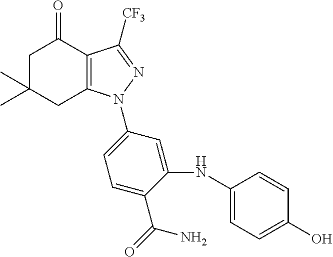 [SMILES: CC1(C)CC(=O)c2c(C(F)(F)F)nn(-c3ccc(C(N)=O)c(Nc4ccc(O)cc4)c3)c2C1] | 143 | | 143 | |
| 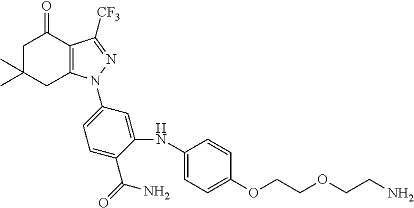 [SMILES: CC1(C)CC(=O)c2c(C(F)(F)F)nn(-c3ccc(C(N)=O)c(Nc4ccc(OCCOCCN)cc4)c3)c2C1] | 144 | | 144 | |
| 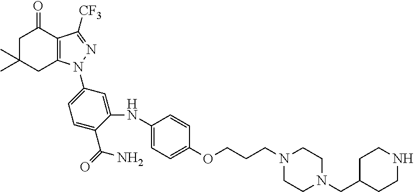 [SMILES: CC1(C)CC(=O)c2c(C(F)(F)F)nn(-c3ccc(C(N)=O)c(Nc4ccc(OCCCN5CCN(CC6CCNCC6)CC5)cc4)c3)c2C1] | 145 | | 145 | |
| 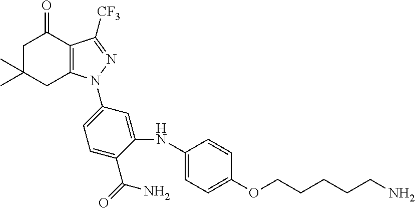 [SMILES: CC1(C)CC(=O)c2c(C(F)(F)F)nn(-c3ccc(C(N)=O)c(Nc4ccc(OCCCCCN)cc4)c3)c2C1] | 146 | | 146 | |
| 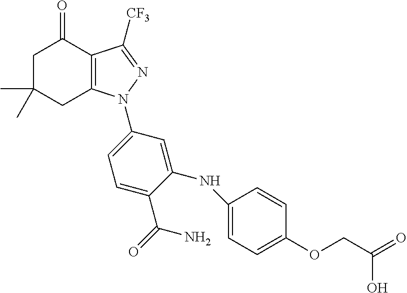 | 147 | 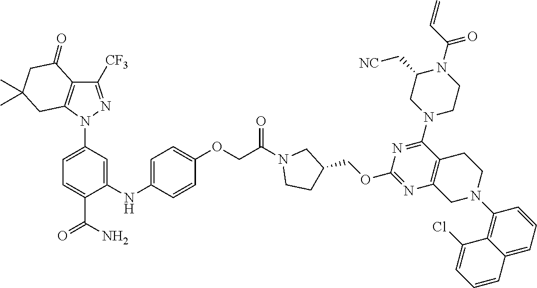 [SMILES: C=CC(=O)N1CCN(c2nc(OC[C@@H]3CCN(C(=O)COc4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N] | 147 | 1070.4 [M + H]+ |
| 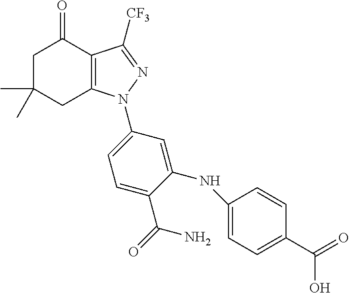 [SMILES: CC1(C)CC(=O)c2c(C(F)(F)F)nn(-c3ccc(C(N)=O)c(Nc4ccc(C(=O)O)cc4)c3)c2C1] | 148 | | 148 | |
| 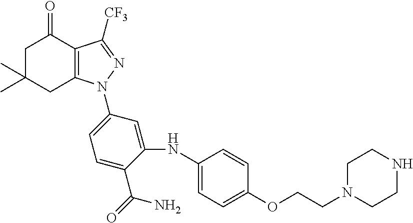 [SMILES: CC1(C)CC(=O)c2c(C(F)(F)F)nn(-c3ccc(C(N)=O)c(Nc4ccc(OCCN5CCNCC5)cc4)c3)c2C1] | 149 | 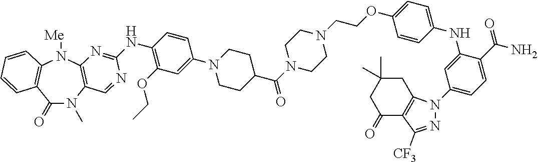 [SMILES: CCOc1cc(N2CCC(C(=O)N3CCN(CCOc4ccc(Nc5cc(-n6nc(C(F)(F)F)c7c6CC(C)(C)CC7=O)ccc5C(N)=O)cc4)CC3)CC2)ccc1Nc1ncc2c(n1)N(C)c1ccccc1C(=O)N2C] | 149 | 1118.8 [M − CF3COOH + H]+ |
| 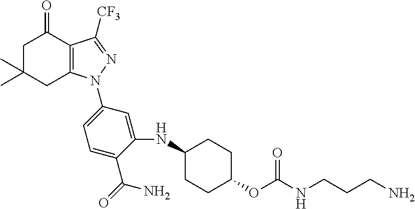 [SMILES: CC1(C)CC(=O)c2c(C(F)(F)F)nn(-c3ccc(C(N)=O)c(N[C@H]4CC[C@H](OC(=O)NCCCN)CC4)c3)c2C1] | 150 | 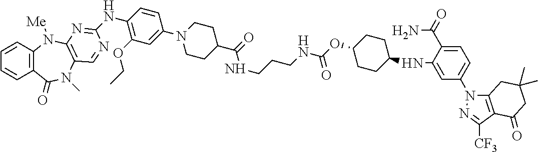 [SMILES: CCOc1cc(N2CCC(C(=O)NCCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(C)c1ccccc1C(=O)N2C] | 150 | 1112.9 [M − CF3COOH + H]+ |
| 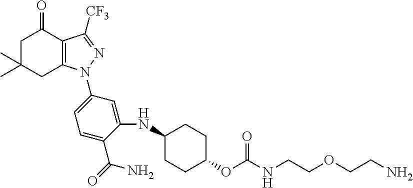 [SMILES: CC1(C)CC(=O)c2c(C(F)(F)F)nn(-c3ccc(C(N)=O)c(N[C@H]4CC[C@H](OC(=O)NCCOCCN)CC4)c3)c2C1] | 151 | 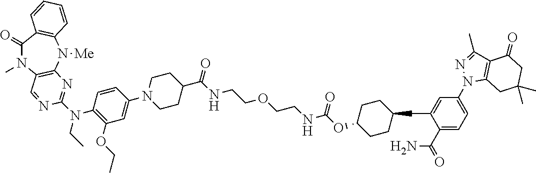 [SMILES: CCOc1cc(N2CCC(C(=O)NCCOCCNC(=O)O[C@H]3CC[C@H](Cc4cc(-n5nc(C)c6c5CC(C)(C)CC6=O)ccc4C(N)=O)CC3)CC2)ccc1N(CC)c1ncc2c(n1)N(C)c1ccccc1C(=O)N2C] | 151 | 1171.5 [M − CF3COOH + H]+ |
| 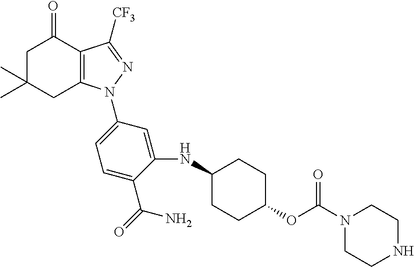 [SMILES: CC1(C)CC(=O)c2c(C(F)(F)F)nn(-c3ccc(C(N)=O)c(N[C@H]4CC[C@H](OC(=O)N5CCNCC5)CC4)c3)c2C1] | 152 | 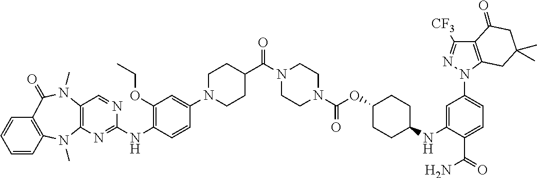 [SMILES: CCOc1cc(N2CCC(C(=O)N3CCN(C(=O)O[C@H]4CC[C@H](Nc5cc(-n6nc(C(F)(F)F)c7c6CC(C)(C)CC7=O)ccc5C(N)=O)CC4)CC3)CC2)ccc1Nc1ncc2c(n1)N(C)c1ccccc1C(=O)N2C] | 152 | 1060.9 [M − CF3COOH + H]+ |
| 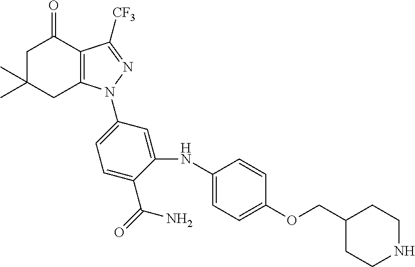 [SMILES: CC1(C)CC(=O)c2c(C(F)(F)F)nn(-c3ccc(C(N)=O)c(Nc4ccc(OCC5CCNCC5)cc4)c3)c2C1] | 153 | 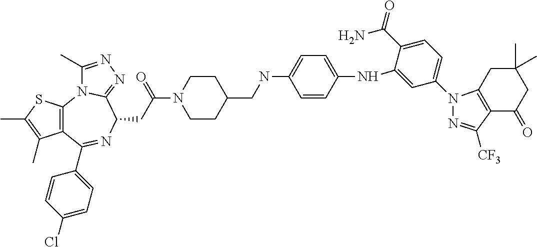 | 153 | 938.2 [M − CF3COOH + H]+ |
| 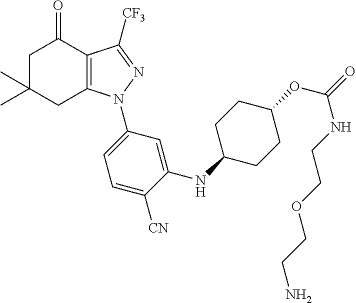 [SMILES: CC1(C)CC(=O)c2c(C(F)(F)F)nn(-c3ccc(C#N)c(N[C@H]4CC[C@H](OC(=O)NCCOCCN)CC4)c3)c2C1] | 154 | 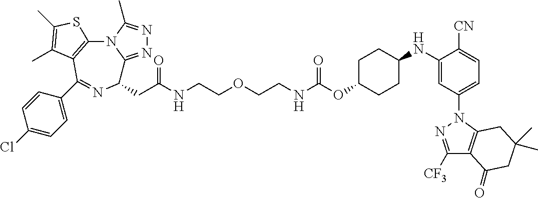 [SMILES: Cc1sc2c(c1C)C(c1ccc(Cl)cc1)=N[C@@H](CC(=O)NCCOCCNC(=O)O[C@H]1CC[C@H](Nc3cc(-n4nc(C(F)(F)F)c5c4CC(C)(C)CC5=O)ccc3C#N)CC1)c1nnc(C)n1-2] | 154 | 959.3 [M − CF3COOH + H]+ |
| 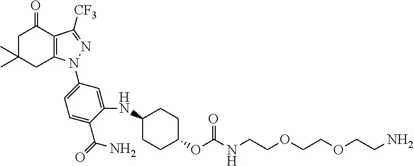 [SMILES: CC1(C)CC(=O)c2c(C(F)(F)F)nn(-c3ccc(C(N)=O)c(N[C@H]4CC[C@H](OC(=O)NCCOCCOCCN)CC4)c3)c2C1] | 155 | 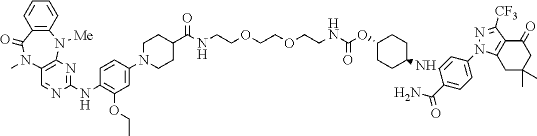 [SMILES: CCOc1cc(N2CCC(C(=O)NCCOCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(C)c1ccccc1C(=O)N2C] | 155 | 1187 [M − CF3COOH + H]+ |
| 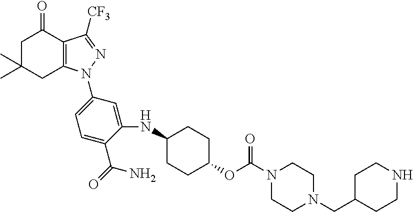 [SMILES: CC1(C)CC(=O)c2c(C(F)(F)F)nn(-c3ccc(C(N)=O)c(N[C@H]4CC[C@H](OC(=O)N5CCN(CC6CCNCC6)CC5)CC4)c3)c2C1] | 156 | 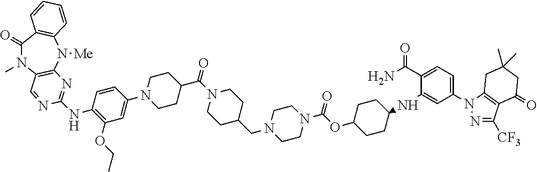 | 156 | 1222.6 [M − CF3COOH + H]+ |
| 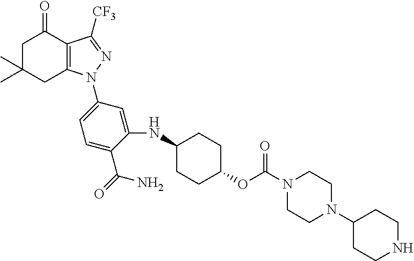 [SMILES: CC1(C)CC(=O)c2c(C(F)(F)F)nn(-c3ccc(C(N)=O)c(N[C@H]4CC[C@H](OC(=O)N5CCN(C6CCNCC6)CC5)CC4)c3)c2C1] | 157 | 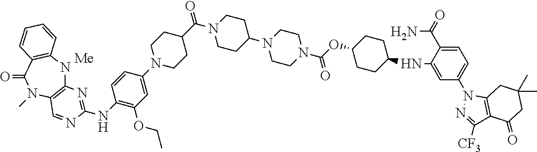 [SMILES: CCOc1cc(N2CCC(C(=O)N3CCC(N4CCN(C(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(C)c1ccccc1C(=O)N2C] | 157 | 1208.6 [M − CF3COOH + H]+ |
| 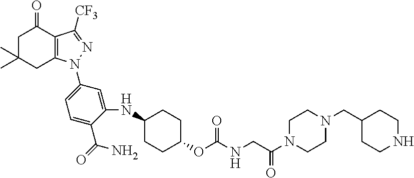 [SMILES: CC1(C)CC(=O)c2c(C(F)(F)F)nn(-c3ccc(C(N)=O)c(N[C@H]4CC[C@H](OC(=O)NCC(=O)N5CCN(CC6CCNCC6)CC5)CC4)c3)c2C1] | 158 | 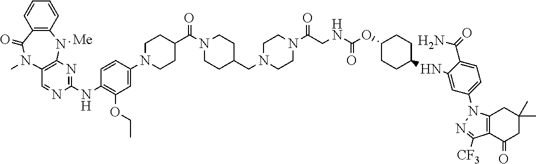 [SMILES: CCOc1cc(N2CCC(C(=O)N3CCC(CN4CCN(C(=O)CNC(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(C)c1ccccc1C(=O)N2C] | 158 | 1279.6 [M − CF3COOH + H]+ |
| 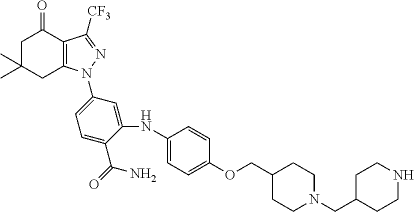 [SMILES: CC1(C)CC(=O)c2c(C(F)(F)F)nn(-c3ccc(C(N)=O)c(Nc4ccc(OCC5CCN(CC6CCNCC6)CC5)cc4)c3)c2C1] | 159 | 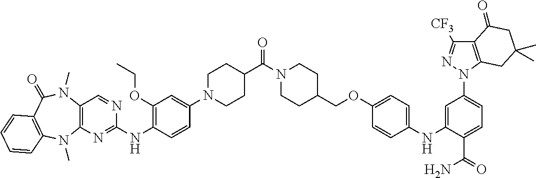 [SMILES: CCOc1cc(N2CCC(C(=O)N3CCC(COc4ccc(Nc5cc(-n6nc(C(F)(F)F)c7c6CC(C)(C)CC7=O)ccc5C(N)=O)cc4)CC3)CC2)ccc1Nc1ncc2c(n1)N(C)c1ccccc1C(=O)N2C] | 159 | 1040.5 [M − CF3COOH + H]+ |
| 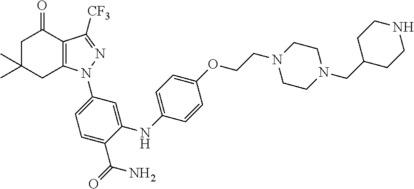 [SMILES: CC1(C)CC(=O)c2c(C(F)(F)F)nn(-c3ccc(C(N)=O)c(Nc4ccc(OCCN5CCN(CC6CCNCC6)CC5)cc4)c3)c2C1] | 160 | 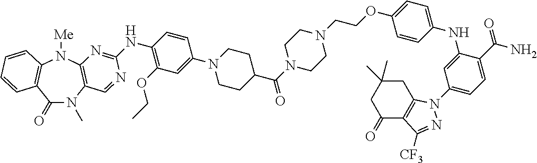 [SMILES: CCOc1cc(N2CCC(C(=O)N3CCN(CCOc4ccc(Nc5cc(-n6nc(C(F)(F)F)c7c6CC(C)(C)CC7=O)ccc5C(N)=O)cc4)CC3)CC2)ccc1Nc1ncc2c(n1)N(C)c1ccccc1C(=O)N2C] | 160 | 1118.8 [M − CF3COOH + H]+ |
| 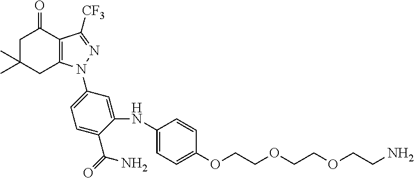 [SMILES: CC1(C)CC(=O)c2c(C(F)(F)F)nn(-c3ccc(C(N)=O)c(Nc4ccc(OCCOCCOCCN)cc4)c3)c2C1] | 161 | 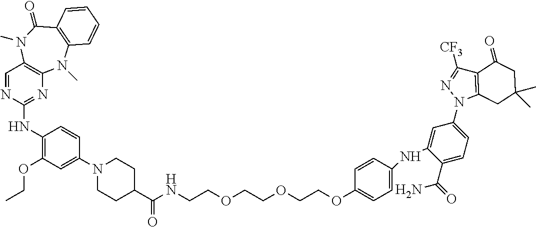 | 161 | 1074.3 [M − CF3COOH + H]+ |
| 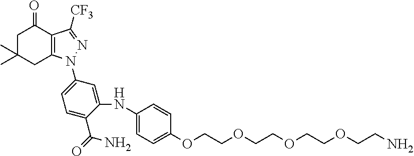 [SMILES: CC1(C)CC(=O)c2c(C(F)(F)F)nn(-c3ccc(C(N)=O)c(Nc4ccc(OCCOCCOCCOCCN)cc4)c3)c2C1] | 162 | 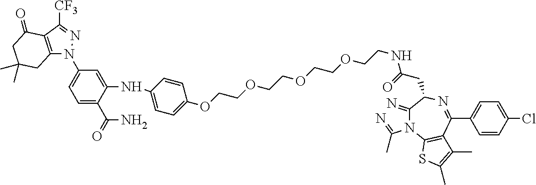 [SMILES: Cc1sc2c(c1C)C(c1ccc(Cl)cc1)=N[C@@H](CC(=O)NCCOCCOCCOCCOc1ccc(Nc3cc(-n4nc(C(F)(F)F)c5c4CC(C)(C)CC5=O)ccc3C(N)=O)cc1)c1nnc(C)n1-2] | 162 | 1016.2 [M − CF3COOH + H]+ |
| 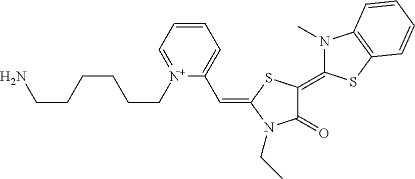 [SMILES: CCn1c(=O)/c(=C2\Sc3ccccc3N2C)s/c1=C\c1cccc[n+]1CCCCCCN] | 163 | 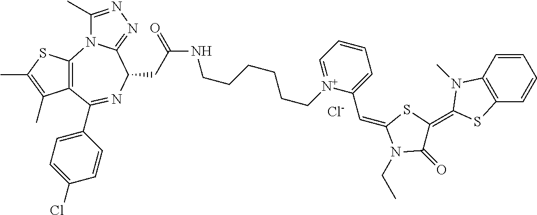 [SMILES: CCn1c(=O)/c(=C2\Sc3ccccc3N2C)s/c1=C\c1cccc[n+]1CCCCCCNC(=O)C[C@@H]1N=C(c2ccc(Cl)cc2)c2c(sc(C)c2C)-n2c(C)nnc21 ; [Cl-]] | 163 | 849.4 [M]+ |
| 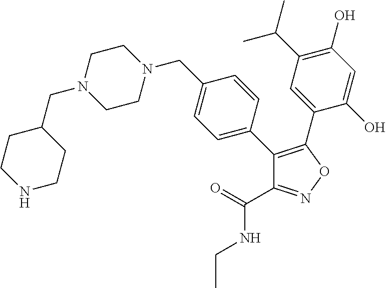 [SMILES: CCNC(=O)c1noc(-c2cc(C(C)C)c(O)cc2O)c1-c1ccc(CN2CCN(CC3CCNCC3)CC2)cc1] | 164 | 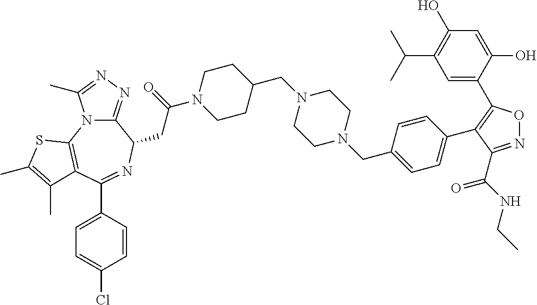 [SMILES: CCNC(=O)c1noc(-c2cc(C(C)C)c(O)cc2O)c1-c1ccc(CN2CCN(CC3CCN(C(=O)C[C@@H]4N=C(c5ccc(Cl)cc5)c5c(sc(C)c5C)-n5c(C)nnc54)CC3)CC2)cc1] | 164 | 944.6 [M − CF3COOH + H]+. |
| 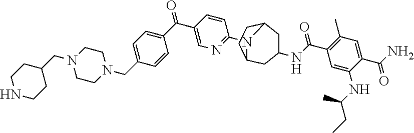 [SMILES: CC[C@@H](C)Nc1cc(C(=O)NC2CC3CCC(C2)N3c2ccc(C(=O)c3ccc(CN4CCN(CC5CCNCC5)CC4)cc3)cn2)c(C)cc1C(N)=O] | 165 | 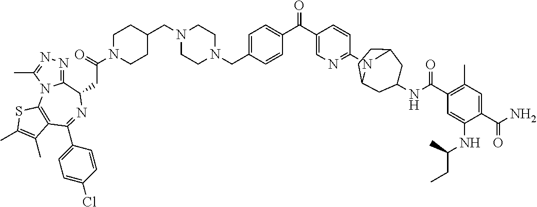 [SMILES: CC[C@@H](C)Nc1cc(C(=O)NC2CC3CCC(C2)N3c2ccc(C(=O)c3ccc(CN4CCN(CC5CCN(C(=O)C[C@@H]6N=C(c7ccc(Cl)cc7)c7c(sc(C)c7C)-n7c(C)nnc76)CC5)CC4)cc3)cn2)c(C)cc1C(N)=O] | 165 | 1117.6 [M − CF3COOH + H]+. |
| 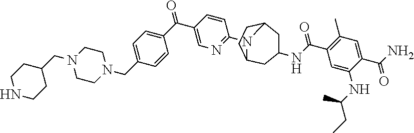 [SMILES: CC[C@@H](C)Nc1cc(C(=O)NC2CC3CCC(C2)N3c2ccc(C(=O)c3ccc(CN4CCN(CC5CCNCC5)CC4)cc3)cn2)c(C)cc1C(N)=O] | 166 | 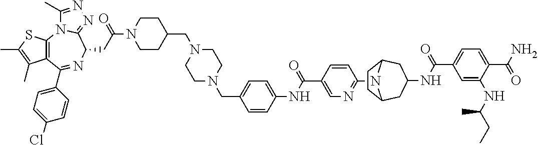 [SMILES: CC[C@@H](C)Nc1cc(C(=O)NC2CC3CCC(C2)N3c2ccc(C(=O)Nc3ccc(CN4CCN(CC5CCN(C(=O)C[C@@H]6N=C(c7ccc(Cl)cc7)c7c(sc(C)c7C)-n7c(C)nnc76)CC5)CC4)cc3)cn2)ccc1C(N)=O] | 166 | 1132.8 [M − HCOOH + H]+. |
| 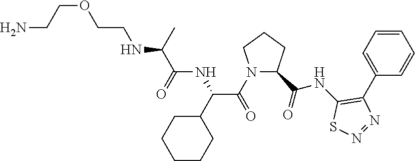 [SMILES: C[C@H](NCCOCCN)C(=O)N[C@H](C(=O)N1CCC[C@H]1C(=O)Nc1snnc1-c1ccccc1)C1CCCCC1] | 167 | 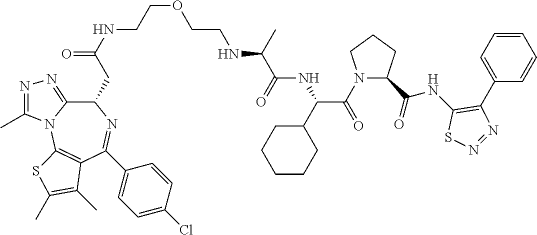 [SMILES: Cc1sc2c(c1C)C(c1ccc(Cl)cc1)=N[C@@H](CC(=O)NCCOCCN[C@@H](C)C(=O)N[C@H](C(=O)N1CCC[C@H]1C(=O)Nc1snnc1-c1ccccc1)C1CCCCC1)c1nnc(C)n1-2] | 167 | 954.2 [M − CF3COOH + H]+. |
| 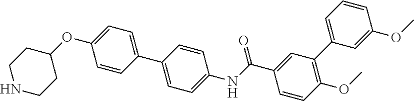 [SMILES: COc1cccc(-c2cc(C(=O)Nc3ccc(-c4ccc(OC5CCNCC5)cc4)cc3)ccc2OC)c1] | 168 | 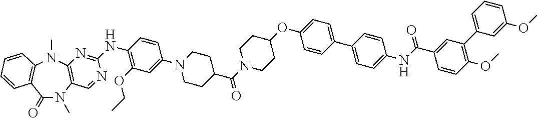 [SMILES: CCOc1cc(N2CCC(C(=O)N3CCC(Oc4ccc(-c5ccc(NC(=O)c6ccc(OC)c(-c7cccc(OC)c7)c6)cc5)cc4)CC3)CC2)ccc1Nc1ncc2c(n1)N(C)c1ccccc1C(=O)N2C] | 168 | 993.1 [M − CF3COOH + H]+ |

TABLE 2
HSP90 binding by Compounds

| | HSP90α | HSP90α |
|---|---|---|
| Compound | binding | binding |
| # | (BODIPY)¹ | (FITC)² |
| 1 | B | B |
| 2 | | B |
| 4 | | C |
| 5 | | B |
| 6 | | B |
| 7 | | B |
| 8 | | C |
| 9 | | B |
| 10A | | B |
| 10B | B | |
| 11 | | B |
| 12 | | B |
| 13 | | B |
| 14 | | B |
| 15 | | A |
| 16 | | A |
| 17 | | A |
| 18 | | A |
| 19 | | A |
| 20 | | B |
| 21 | | B |
| 22 | | A |
| 23 | | B |
| 24 | | B |
| 25 | | B |
| 26 | | B |
| 27 | | B |
| 28 | | B |
| 29 | | B |
| 30 | | B |
| 31 | | B |
| 32 | | B |
| 33 | | B |
| 34 | | B |
| 35 | | B |
| 36 | | B |
| 37 | | B |
| 38 | | B |
| 39 | | B |
| 40 | C | C |
| 41 | | A |
| 42 | | B |
| 43 | | C |
| 44 | | B |
| 46 | | B |
| 47 | | A |
| 48 | | B |
| 49 | | B |
| 50 | | B |
| 51 | | B |
| 52 | | B |
| 53 | | B |
| 54 | | B |
| 55 | A | |
| 56 | A | |
| 57 | B | |
| 58 | B | |
| 59 | A | |
| 60 | A | |
| 61 | B | |
| 62 | C | |
| 63 | B | |
| 64 | A | |
| 65 | B | |
| 66 | A | |
| 67 | B | |
| 68 | B | |
| 69 | B | |
| 70 | B | |
| 71 | C | |
| 72 | B | |
| 73 | B | |
| 74 | A | |
| 75 | A | |
| 76 | A | |
| 77 | B | |
| 78 | A | |
| 79 | B | |
| 80 | B | |
| 81 | B | |
| 82 | A | |
| 83 | C | |
| 84 | C | |
| 85 | C | |
| 86 | C | |
| 87 | B | |
| 88 | C | |
| 89 | B | |
| 90 | A | |
| 91 | A | |
| 92 | A | |
| 93 | B | |
| 94 | B | |
| 95 | B | |
| 96 | B | |
| 97 | B | |
| 98 | B | |
| 99 | C | |
| 100 | B | |
| 101 | A | |
| 102 | A | |
| 103 | B | |
| 104 | A | |
| 105 | B | |
| 106 | B | |
| 107 | B | |
| 108 | C | |
| 109 | B | |
| 110 | | B |
| 111 | | B |
| 112 | | B |
| 113 | | B |
| 114 | | B |
| 116 | | B |
| 117 | B | |
| 119 | C | |
| 120 | B | |
| 121 | C | |
| 122 | B | |
| 123 | C | |
| 124 | B | |
| 125 | C | |
| 126 | B | |
| 127 | B | |
| 128 | B | |
| 129 | B | |
| 130 | B | |
| 131 | C | |
| 132 | C | |
| 133 | C | |
| 147 | C | |
| 149 | B | |
| 150 | C | |
| 151 | C | |
| 152 | C | |
| 153 | C | |
| 154 | C | |
| 155 | C | |
| 156 | C | |
| 157 | C | |
| 158 | C | |
| 159 | C | |
| 160 | B | |
| 161 | | C |
| 162 | C | |
| 163 | | C |
| 164 | | B |
| 165 | C | C |
| 166 | | C |
| 167 | C | |

¹HSP90α-binding FP (BODIPY) assay: A. IC50 < 100 nM; B. IC50 = 100-1000 nM; C. IC50 > 1000 nM
²HSP90α-binding FP (FITC) assay: A. IC50 < 100 nM; B. IC50 = 100-1000 nM; C. IC50 > 1000 nM

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound of the Formula:
H-L-T;
or a pharmaceutically acceptable salt thereof, wherein
H is an HSP90, KRAS, or ERK5 binder;
L is a linker; and
T is a target protein binder.

2. The compound of claim 1, or a pharmaceutically acceptable salt thereof, wherein H is selected from 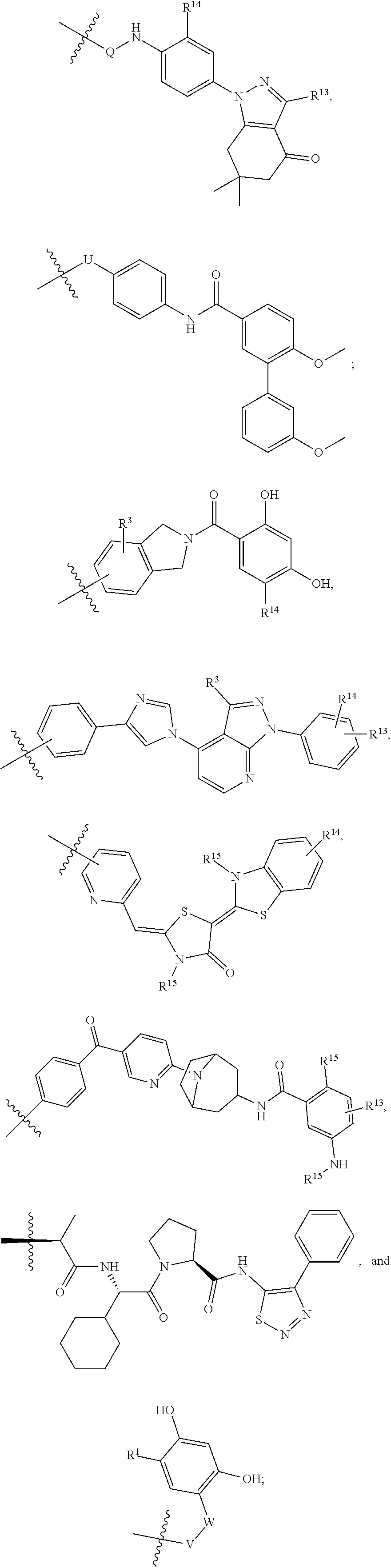
wherein
Q and U are each independently selected from phenyl, heteroaryl, heterocyclyl, and cycloalkyl, each of which being optionally substituted with 1 to 3 groups selected from R²;
R¹³ and R¹⁴ are each independently selected from hydrogen, halo, —CN, (C₁-C₄)alkyl, halo(C₁-C₄)alkyl, and —C(O)NR^{a}R^{b};
R¹⁵ is hydrogen, (C₁-C₄)alkyl, or halo(C₁-C₄)alkyl;
W is 5- or 6-membered heteroaryl optionally substituted with 1 to 3 groups selected from R²;
V is phenyl or 5- to 9-membered heteroaryl optionally substituted with 1 to 3 groups selected from R³;
R¹ is halo, (C₁-C₄)alkyl, halo(C₁-C₄)alkyl, (C₁-C₄)alkoxy, or halo(C₁-C₄)alkoxy;
R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄alkylene)NR^{a}R^{b}, —C₁₋₄alkylNR^{a}R^{b}, —SR^{a}, —S(O)R^{a}, —S(O)₂R^{a}, —S(O)NR^{a}R^{b}, —SO₂NR^{a}R^{b}, —NR^{a}(C₁₋₄alkyl)OR^{a}, —SH, —S(C₁₋₄alkyl), —NR^{a}(C₁₋₄alkyl)NR^{a}R^{b}, —C₁₋₆alkylC(O)NR^{a}R^{b}, —O(C₁₋₄alkylene)NR^{a}C(O)(C₁₋₄alkylene)NR^{a}R^{b}, phenyl or 5- to 7-membered heteroaryl, wherein said phenyl and 5- to 7-membered heteroaryl are each optionally and independently substituted with 1 to 3 groups selected from R⁴;
R^{a} and R^{b} are each independently selected from hydrogen and (C₁-C₄)alkyl, wherein said (C₁-C₄)alkyl is optionally substituted with one or more halo or a 3- to 7-membered heterocyclyl, or both; and
R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy.

3. The compound of claim 1, or a pharmaceutically acceptable salt thereof, wherein H is

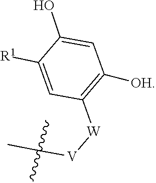

4. The compound of claim 1, or a pharmaceutically acceptable salt thereof, wherein H is selected from 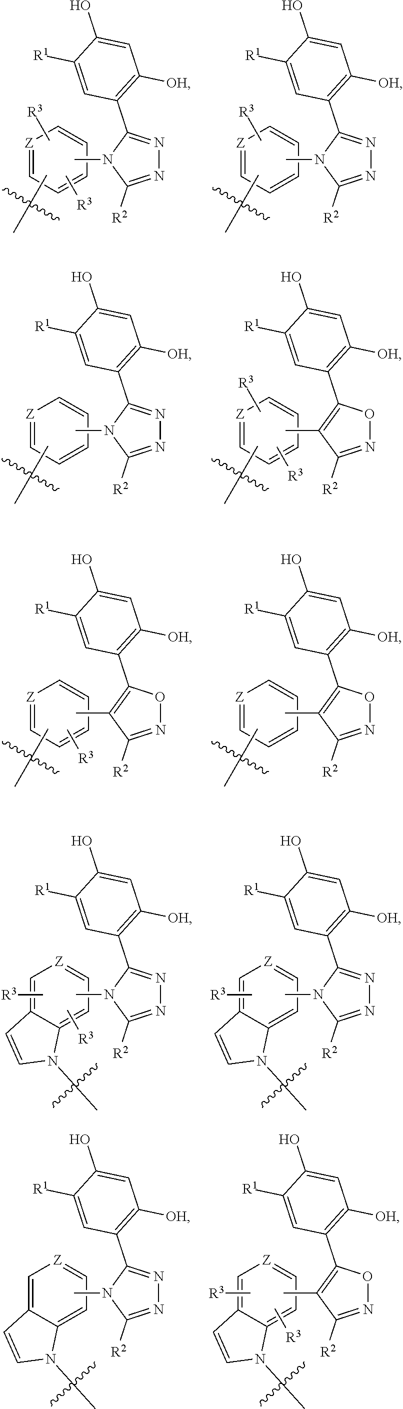 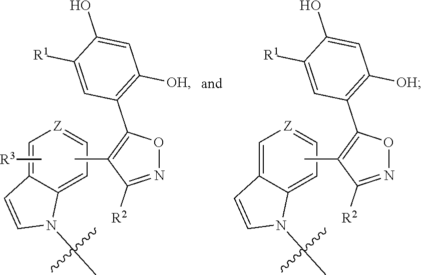
and
Z is N or CH.

5. (canceled)

6. The compound of claim 2, wherein each R³ is independently (C₁-C₄)alkyl or halo.

7. (canceled)

8. The compound of claim 1, or a pharmaceutically acceptable salt thereof, wherein H is

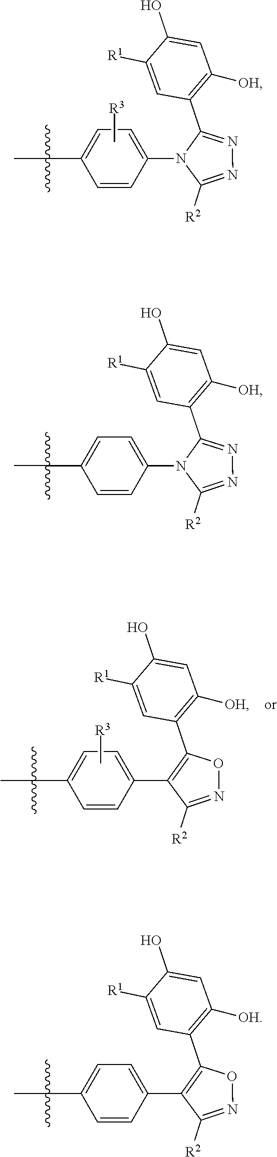

9. (canceled)

10. The compound of claim 1, or a pharmaceutically acceptable salt thereof, wherein H is

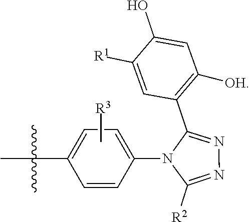

11. The compound of claim 2, or a pharmaceutically acceptable salt thereof, wherein R¹ is halo or (C₁-C₄)alkyl.

12. (canceled)

13. (canceled)

14. The compound of claim 2, or a pharmaceutically acceptable salt thereof, wherein R² is —OR^{a}, —SR^{a}, —C(O)NR^{a}R^{b}, or —C(O)NR^{a}(C₁-4alkylene)NR^{a}R^{b}.

15. The compound of claim 2, or a pharmaceutically acceptable salt thereof, wherein R^{a} and R^{b} are each independently selected from hydrogen and (C₁-C₄)alkyl, wherein said (C₁-C₄)alkyl is optionally substituted with 1 to 3 halo or a 6-membered heterocyclyl.

16-18. (canceled)

19. The compound of claim 1, or a pharmaceutically acceptable salt thereof, wherein
L is selected from -Het¹-X¹-*, -Het¹-, -Het¹-Het²-X¹-*, *-Het¹-Het²-, —NR^{d}—(CH₂)ₘ—X³—NR^{c}—CH₂)ₘ-Het¹-X¹-Het²-X²-*, —NR^{c}—(CH₂)ₘ-Het¹-X¹-Het²-X²-*, -Het¹-X¹-Het²-X²-*, *O—(CH₂)ₘ—NR^{c}—X¹—(CH₂)ₘ—NR^{d}—, *-X¹—NR^{c}—X²—O—(CH₂)ₘ—NR^{d}—, *-X¹-Het¹-X²-Het²-(CH₂)ₘO—, *O-Het¹-, *O-Het¹-X¹—, *-X¹(OCH₂CH₂)ₙ—NR^{c}—, *-(CH₂)ₘNR^{c}—, —(CH₂)ₘ—, —O—, *X¹NR^{c}—, —NR^{c}—(CH₂)ₘ—X¹-Het¹-X²-*, —NR^{d}—(CH₂)ₘ—X³—NR^{c}—(CH₂)ₘ-Het¹-X¹-Het²-X²-*, *O-Het¹-X¹—(CH₂)ₘ—NR^{d}—, *-X¹—NR^{c}—X²—(CH₂)ₘ—NR^{d}—, *X¹-Het¹-X²—NR^{c}—X³-Het²-(OCH₂CH₂)ₙ—(CH₂)ₘ—NR^{d}—(CH₂)ₘ—, —NR^{d}—(CH₂)ₘ—X¹—NR^{c}—(CH₂CH₂O)ₙ-*, —NR^{c}—(CH₂)ₘ—X¹—NR^{c}—(CH₂)ₚ-*, *X¹-Het¹-X²—NR^{c}—X³-Het²-(OCH₂CH₂)ₙ—NR^{d}—(CH₂)ₘ—, —NR^{c}—(CH₂)ₘ—X¹-Het¹-X²-Het²-X³-*, *O—X¹-Het¹-, —O(CH₂)ₘ—X¹-Het¹-X²-Het²-X³-*, —O(CH₂)ₘ—X¹—NR^{c}—(CH₂)ₚ-Het¹-X²-Het²-X³-*, *O—(CH₂)ₘ—NR^{c}—, *O—X¹-Het¹-X²—, *-X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH)CH₃-Het¹-X²-Het³-X³-*, —NR^{c}—(CH₂)ₘ—X¹—(CH₂)ₚ-Het¹-X²-Het²-X³-*, —NR^{c}—(CH₂)ₘ—X¹—NR^{d}—(CH₂)ₚ-Het¹-X²-Het²-X³-*, —NR^{c}—(CH₂)ₘ—NR^{d}—X¹-Het¹-X²-*, *Het¹-X¹-Het²-X²—, *-Het¹-X¹-Het²-X²—O—, —O(CH₂)ₘ-Het¹-(CH₂)ₚ—O(CH₂)ₘ—NR^{c}—X²-*, *-O(CH₂)ₘ-Het¹-(CH₂)ₚ—O(CH₂)ₘ—NR^{c}—X²—, *-Het¹-O—O—(CH₂)ₘ—X¹-Het²-X²—, *-Het¹-O—(CH₂)ₘ—X¹—NR^{c}—(CH₂CH₂O)ₙ(CH₂)ₘ-Het²-X²—, *-Het¹-X¹—NR^{c}—(CH₂)ₘ—, *-Het¹-X¹-Het²-Het³-X²—, *-Het¹-X¹—NR^{c}—(CH₂CH₂O)ₙ(CH₂)ₘ—, *-Het¹-X¹—NR^{c}—(CH₂CH₂O)ₙHet²-(CH₂)ₘ—X²—, *-Het¹-X¹—NR^{c}—(CH₂CH₂O)ₙ—, *-Het¹-X¹—NR^{c}—(CH₂)ₘ-Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³-, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ-Het³-(CH₂)ₚ—NR^{c}—(CH₂)ₘ—, *-Het¹-X¹-Het²-(CH₂CH₂O)ₙ—, *-Het¹-X¹—(CH₂)ₘ-Het²-X²—, *-(CH₂CH₂O)ₒ—(CH₂)ₚ-Het¹-X¹-Het²-(CH₂CH₂O)ₙ—, *-(CH₂CH₂O)ₙ—(CH₂)ₘ-Het¹-X¹-Het²-X², *-Het¹-X¹-Phe-X²—NR^{c}—X³—, *-(CH₂CH₂O)ₒ—(CH₂)ₚ-Het¹-X¹-Phe-X²—NR^{c}—(CH₂CH₂O)ₙ—, *-(CH₂CH₂O)ₙ—(CH₂)ₘ—NR^{c}-Phe-X¹—, *-(CH₂CH₂O)ₒ—(CH₂)ₚ—NR^{c}-Phe-(CH₂CH₂O)ₙ—, *-(CH₂CH₂O)ₒ—(CH₂)ₚ—NR^{c}—(CH₂CH₂O)ₙ—(CH₂)ₘ—, *-(CH₂C₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹-Het²-X²—, *-(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, *-NR^{c}—(CH₂CH₂O)ₙ—(CH₂)ₘ-Phe-NH—X¹-Het¹-X², *-NR^{c}—(CH₂CH₂O)ₙ—(CH₂)ₘ-Phe-NH—X¹-Het¹-X²—(CH₂CH₂O)ₒ, *-(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, *-(CH₂CH₂O)ₒ—(CH₂)ₚ—NR^{c}—(CH₂CH₂O)ₙ—(CH₂)ₘ-Het¹-X¹—, *-(CH₂CH₂O)ₒ—(CH₂)ₚ—NR^{c}—(CH₂CH₂O)ₙ—(CH₂)ₘ-Het¹-X¹—(CH₂CH₂O)ₙ—, *-(CH₂CH₂O)ₙ—(CH₂)ₘ—NR^{c}—(CH₂)ₘ—C(O)—NR^{d}-Het¹-X l -Het²-(CH₂CH₂O)ₒ—(CH₂)ₚ, or *-NR^{c}—(CH₂)ₘ—C(O)—NR^{d}—(CH₂)ₘ-Het¹-X¹-Het²-X²—, *C(O)O—, *-X¹-Het¹-(CH₂CH₂O)ₒ—(CH₂)ₘ—NR^{c}—, -Het¹-(CH₂)ₘ-Het²-, *-Het¹-X¹-Het²-(CH₂)ₚ—O—(CH₂)ₘ-*, *O(CH₂)ₘC(O), *-OC(O)—NR^{c}—(CH₂)ₘ—NR^{d}—, *-OC(O)—NR^{c}—(CH₂)ₘ—O—(CH₂)ₘ—NR^{d—}, *OC(O)Het¹, *-OC(O)—NR^{c}—(CH₂CH₂O)ₒ—NR^{d}—, *OC(O)Het¹-Het²-, *-OC(O)—NR^{c}—(CH₂)ₘC(O)-Het¹-X¹-Het²-, *O—(CH₂)ₘ-Het¹-, and *O—(CH₂)ₘ-Het¹-X¹-Het²;
the * indicates the point of attachment to H,
Het¹, Het², and Het³ are each independently phenyl, a 4- to 6-membered heterocyclyl, 5-to 7-membered heteroaryl, or a 4- to 6-membered cycloalkyl, each of which are optionally substituted with (C₁-C₄)alkyl;
X¹, X², and X³, are each independently C(O) or (CH₂)ᵣ;
R^{c} and R^{d} are each independently hydrogen, (C₁-C₄)alkyl, or halo(C₁-C₄)alkyl; and
m, n, o, p, q and r are each independently integers selected from 0, 1, 2, 3, 4, 5, and 6.

20. (canceled)

21. The compound of claim, or a pharmaceutically acceptable salt thereof, wherein Het¹ and Het² are each independently phenyl or a 4- to 6-membered heterocyclyl.

22. (canceled)

23. (canceled)

24. The compound of claim 1, or a pharmaceutically acceptable salt thereof, wherein L is selected from

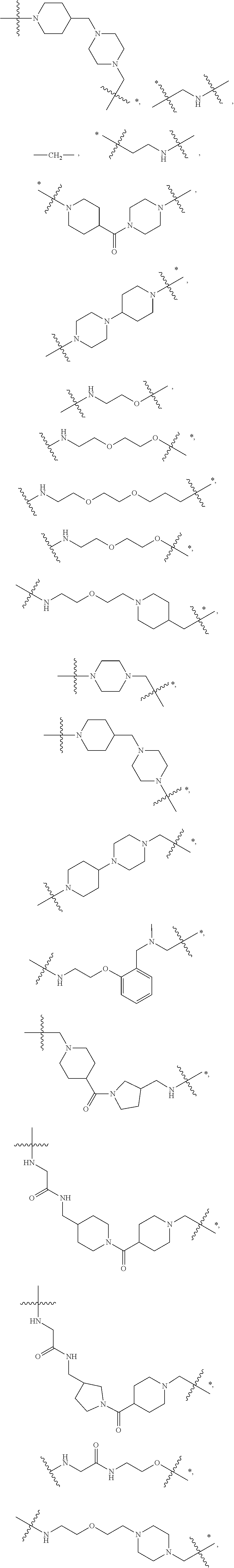

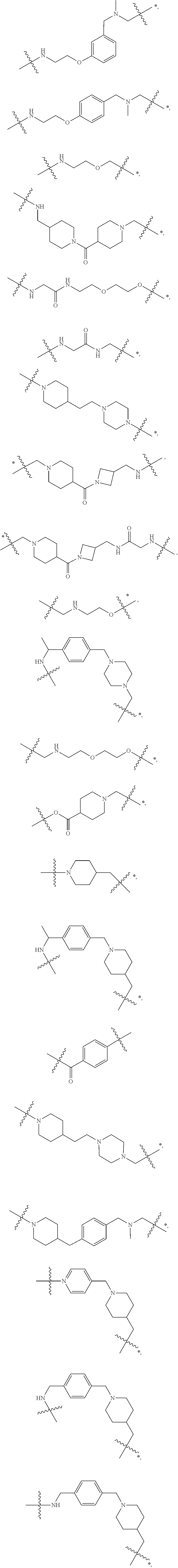

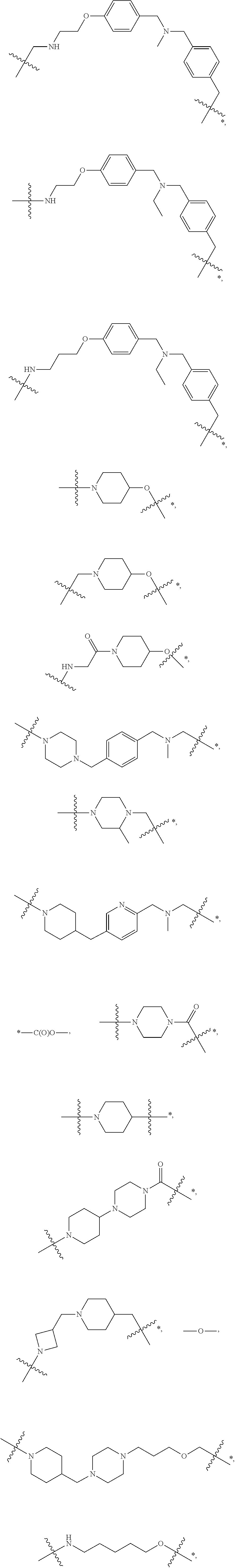

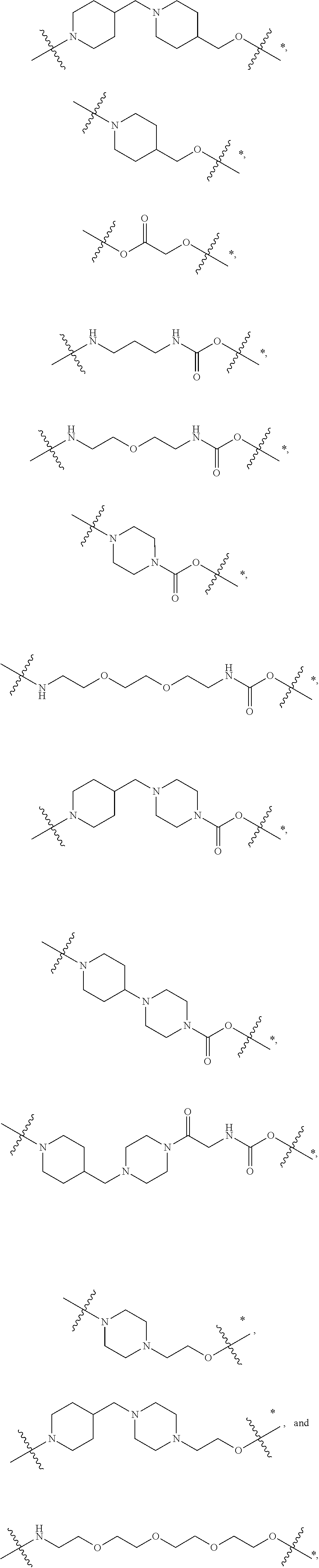

25. The compound of claim 1, or a pharmaceutically acceptable salt thereof, wherein the target protein binder is a binder of BET.

26. The compound of claim 1, or a pharmaceutically acceptable salt thereof, wherein the target protein binder is of the Formula: 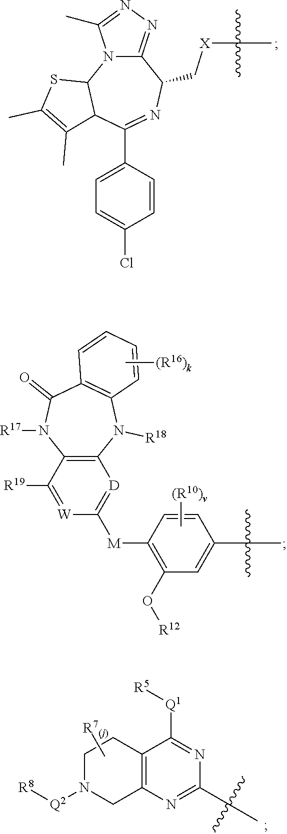
wherein
X is C(O) or (C₁-C₄)alkylene; Q¹ is a nitrogen containing heteroaryl or heterocyclyl ring, each of which are optionally substituted with 1 to 3 groups selected from R⁶;
R⁵ is —C(O)Y or —S(O)₂Y;
Y is a (C₁-C₆)alkyl, halo(C₁-C₆)alkyl, (C₂-C₆)alkenyl, halo(C₂-C₆)alkenyl, NH₂, —NH(C₁-C₆)alkyl, —N[(C₁-C₆)alkyl]₂, NHNH₂, or NHOH, wherein said (C₂-C₆)alkenyl, alone or as recited in halo(C₂-C₆)alkenyl, is optionally substituted with (C₁-C₆)alkyl, halo(C₁-C₆)alkyl, heteroalkyl, hydroxy(C₁-C₆)alkyl, —C(O)NH₂, —C(O)NH(C₁-C₆)alkyl, or —C(O)N[(C₁-C₆)alkyl]₂;
R⁶ is (C₁-C₆)alkyl, halo(C₁-C₆)alkyl, (C₁-C₆)alkoxy, halo(C₁-C₆)alkoxy, hydroxy(C₁-C₆)alkyl, cyano(C₁-C₆)alkyl, oxo, cyano, heteroalkyl, —C(O)OH, —C(O)O(C₁-C₆)alkyl, —C(O)NH₂, —C(O)NH(C₁-C₆)alkyl, or —C(O)N[(C₁-C₆)alkyl]2, wherein said (C₁-C₆)alkyl is optionally substituted with heteroaryl;
R⁷ is halo, hydroxyl, (C₁-C₆)alkyl, halo(C₁-C₆)alkyl, (C₁-C₆)alkoxy, halo(C₁-C₆)alkoxy, cylcoalkyl, heteroalkyl, hydroxy(C₁-C₆)alkyl, or S(C₁-C₆)alkyl;
j is 1 or 2;
Q² is a bond, —C(O)—, or (C₁-C₃)alkylene;
R⁸ is cycloalkyl, heterocyclyl, aryl, or heteroaryl, each of which are optionally substituted with 1 to 3 groups selected from R⁹;
R⁹ is halo, (C₁-C₆)alkyl, (C₂-C₆)alkenyl, halo(C₂-C₆)alkenyl, (C₂-C₆)alkynyl, oxo, cyano, —(C₁-C₆)alkylOR^{c}, —(C₁-C₆)alkylN(R^{d})₂, —(C₁-C₆)alkylC(O)OR^{d}, OH, —(C₁-C₆)alkylC(O)N(R^{d})₂, —(C₁-C₆)alkylO(C₁-C₆)alkylN(R^{d})₂, —(C₁-C₆)alkyl SOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, or CN, wherein each aryl, cycloalkyl, heterocyclyl, and heteoaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e}; and
R^{e} is selected from halo, oxo, CN, NO₂, —N(R^{d})₂, —OR^{d}, —C(O)OR^{d}, (C₁-C₆)alkyl, —(C₁-C₆)alkylOR^{c}, halo(C₁-C₆)alkyl, (C₁-C₆)alkoxy, halo(C₁-C₆)alkoxy, —(C₁-C₆)alkylC(O)OR^{d}, —(C₁-C₆)alkylC(O)N(R^{d})₂, (C₂-C₆)alkenyl, halo(C₂-C₆)alkenyl, (C₂-C₆)alkynyl, —(C₁-C₆)alkylSR^{d}, —(C₁-C₆)alkylOR^{c}, —(C₁-C₆)alkylN(R^{d})₂, —C(O)N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, aryl, heteroaryl, cycloalkyl, and heterocycloalkyl.
R¹⁰, R¹⁶, and R¹⁹ are each independently selected from halo, (C₁-C₆)alkyl, (C₂-C₆)alkenyl, halo(C₂-C₆)alkenyl, (C₂-C₆)alkynyl, —(C₁-C₆)alkylOR^{c}, —(C₁-C₆)alkylN(R^{d})₂, —(C₁-C₆)alkylC(O)OR^{d}, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, and CN, wherein each aryl, cycloalkyl, heterocyclyl, and heteoaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e};
W and D are each independently N or CR²⁰;
M is O, S, or NR¹¹;
R¹¹, R¹⁷, R¹⁸, and R²⁰, are each independently selected from hydrogen, (C₁-C₆)alkyl, and S(O)₂(C₁-C₆)alkyl;
R¹² is hydrogen, (C₁-C₆)alkyl, halo(C₁-C₆)alkyl, —(C₁-C₆)alkylOR^{c}, S(O)₂(C₁-C₆)alkyl, aryl, heteroaryl, cycloalkyl, heterocycloalkyl, C(O)(C₁-C₆)alkyl, or —(C₁-C₆)alkylaryl, wherein each aryl, cycloalkyl, heterocyclyl, and heteoaryl alone and in connection with —(C₁-C₆)alkylaryl are optionally substituted with 1 to 3 groups selected from Re;
and
k and v are each independently 0, 1, 2, or 3.

27-50. (canceled)

51. The compound of claim 1, wherein the compound is selected from the following structural formula: 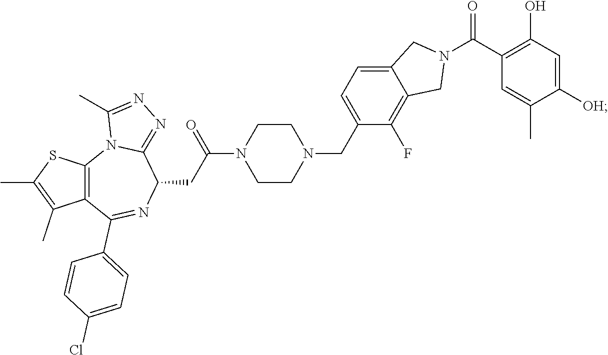 [SMILES: Cc1cc(C(=O)N2Cc3ccc(CN4CCN(C(=O)C[C@@H]5N=C(c6ccc(Cl)cc6)c6c(sc(C)c6C)-n6c(C)nnc65)CC4)c(F)c3C2)c(O)cc1O] 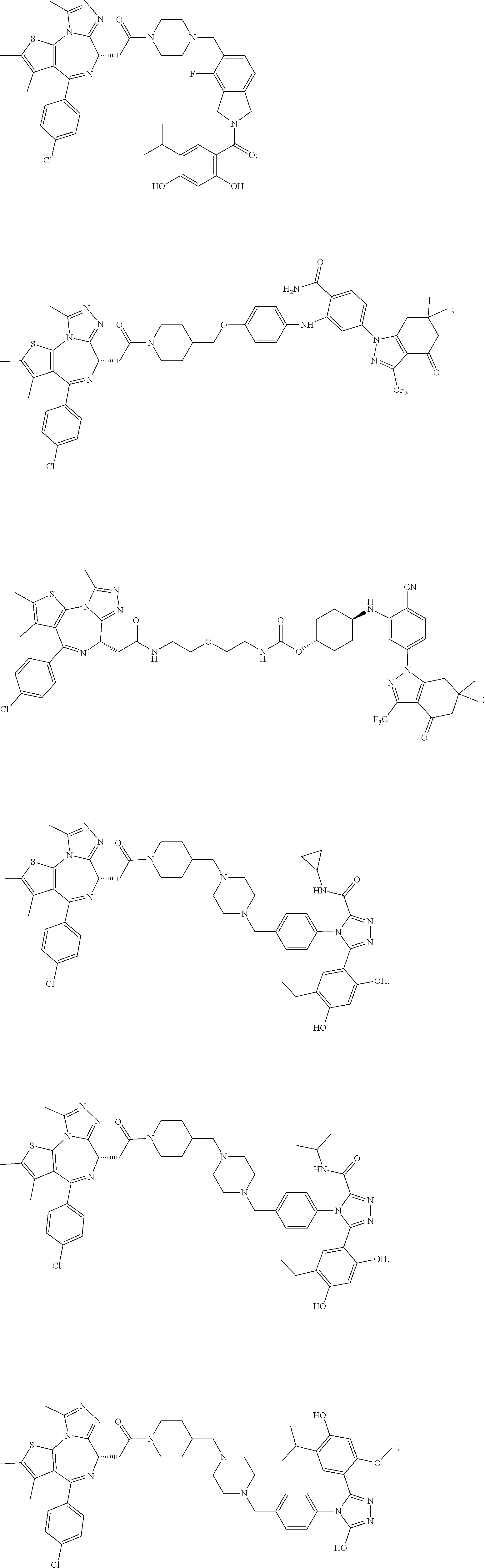 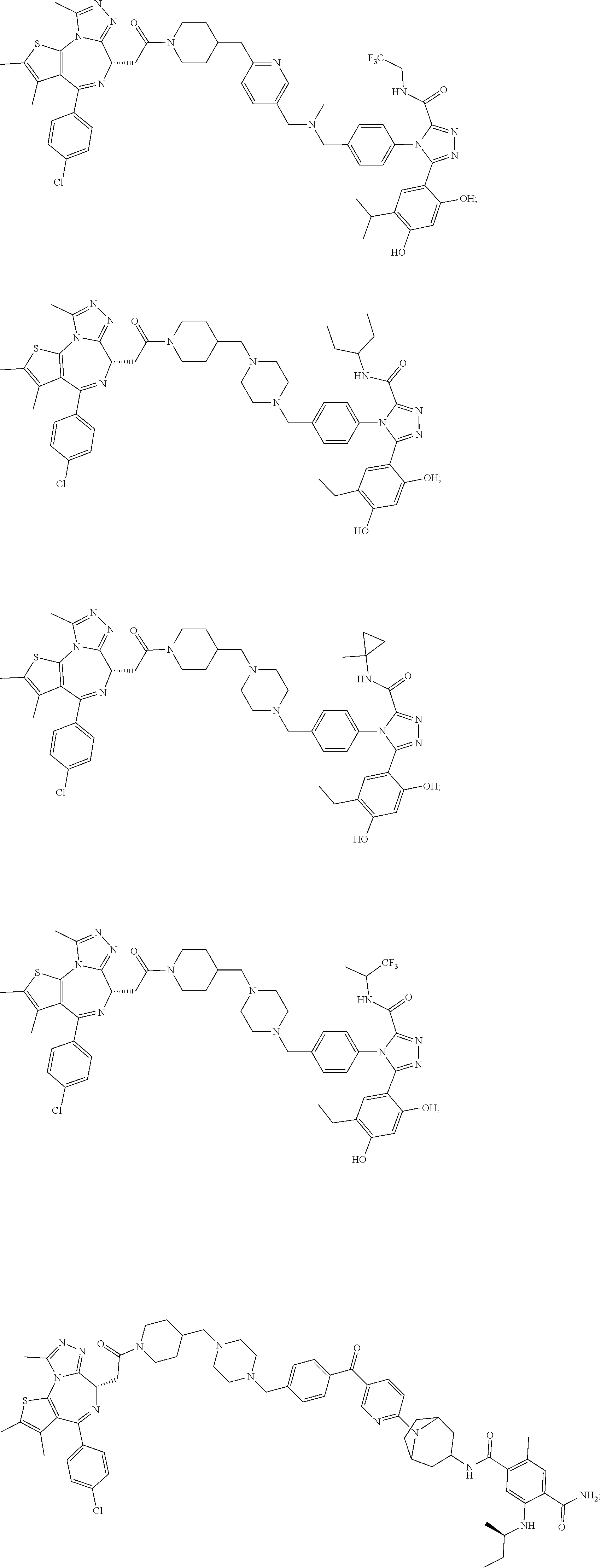 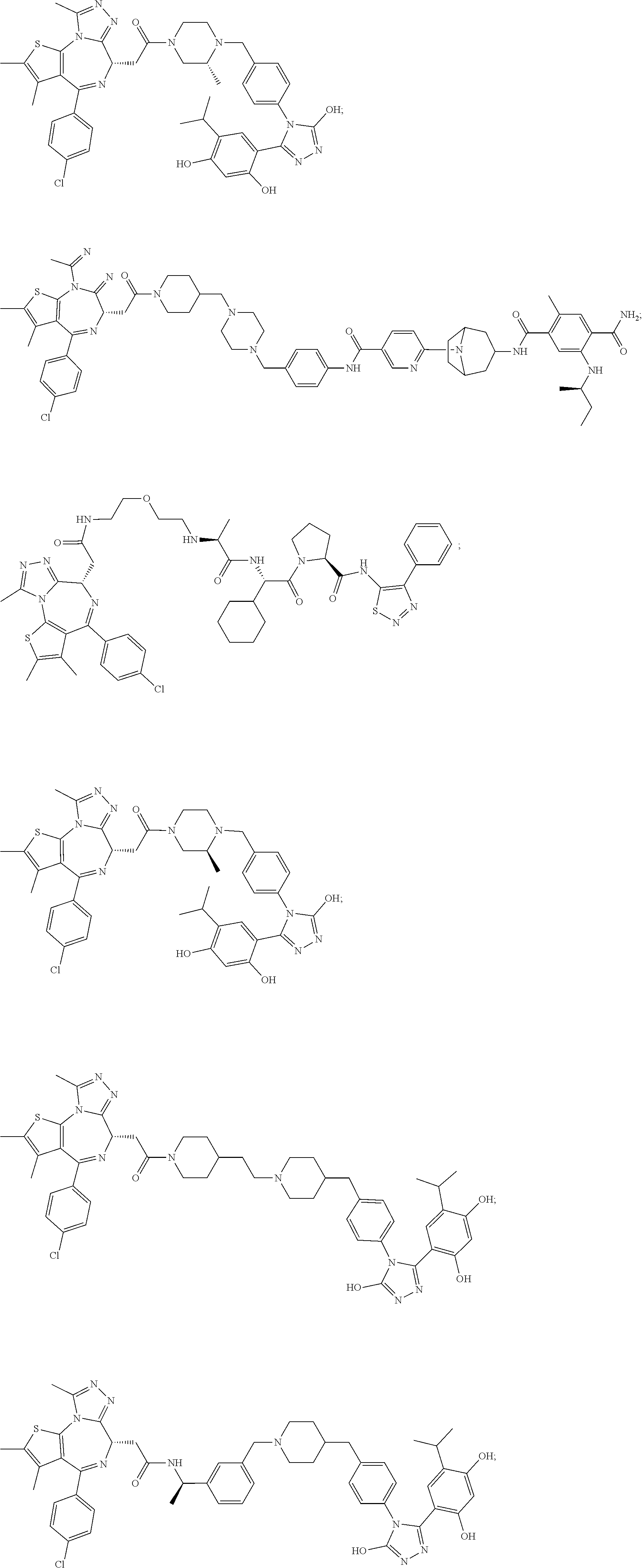 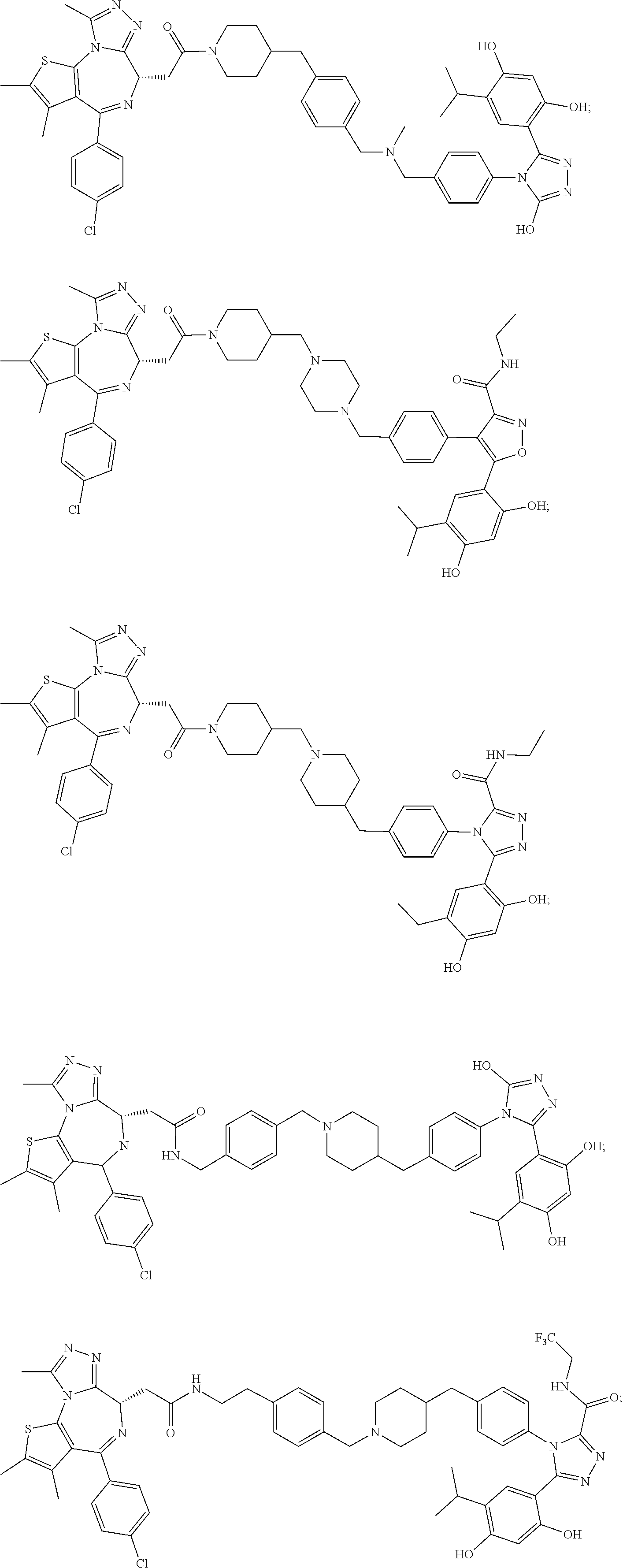 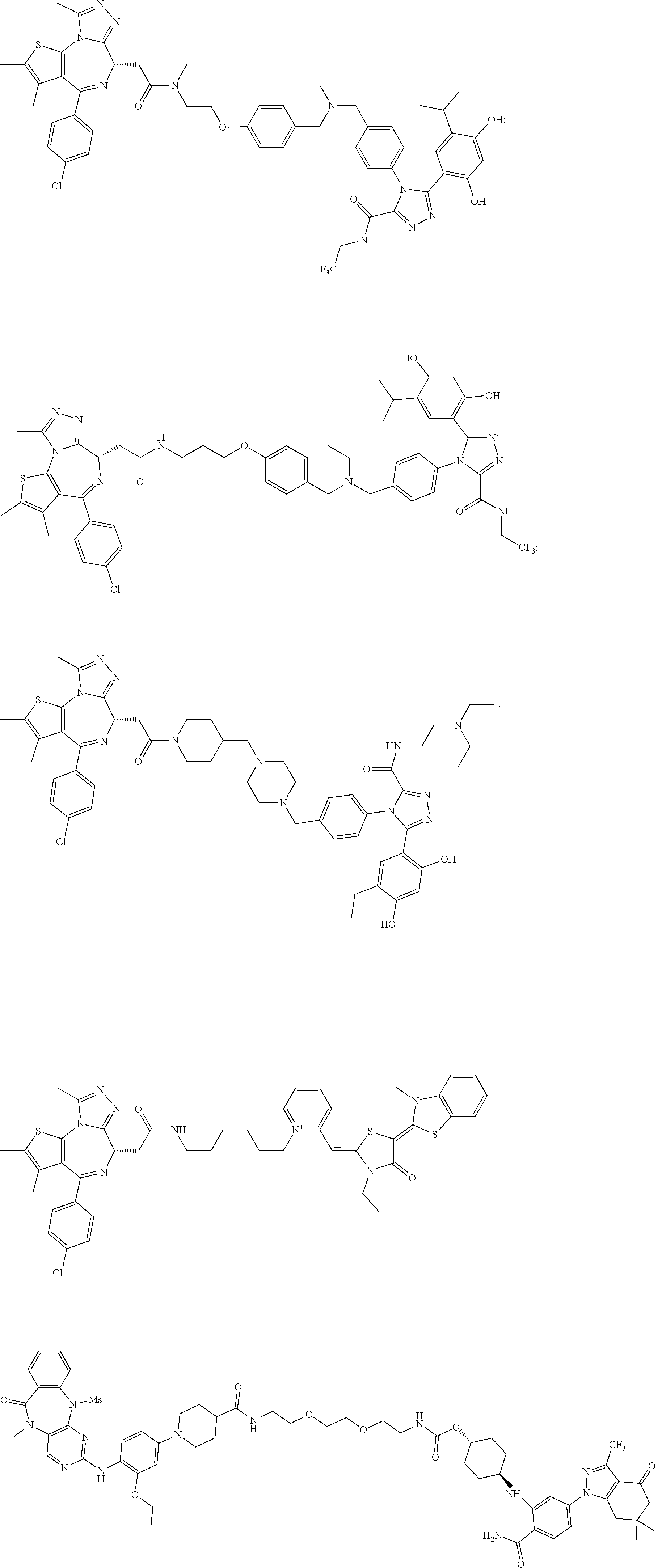 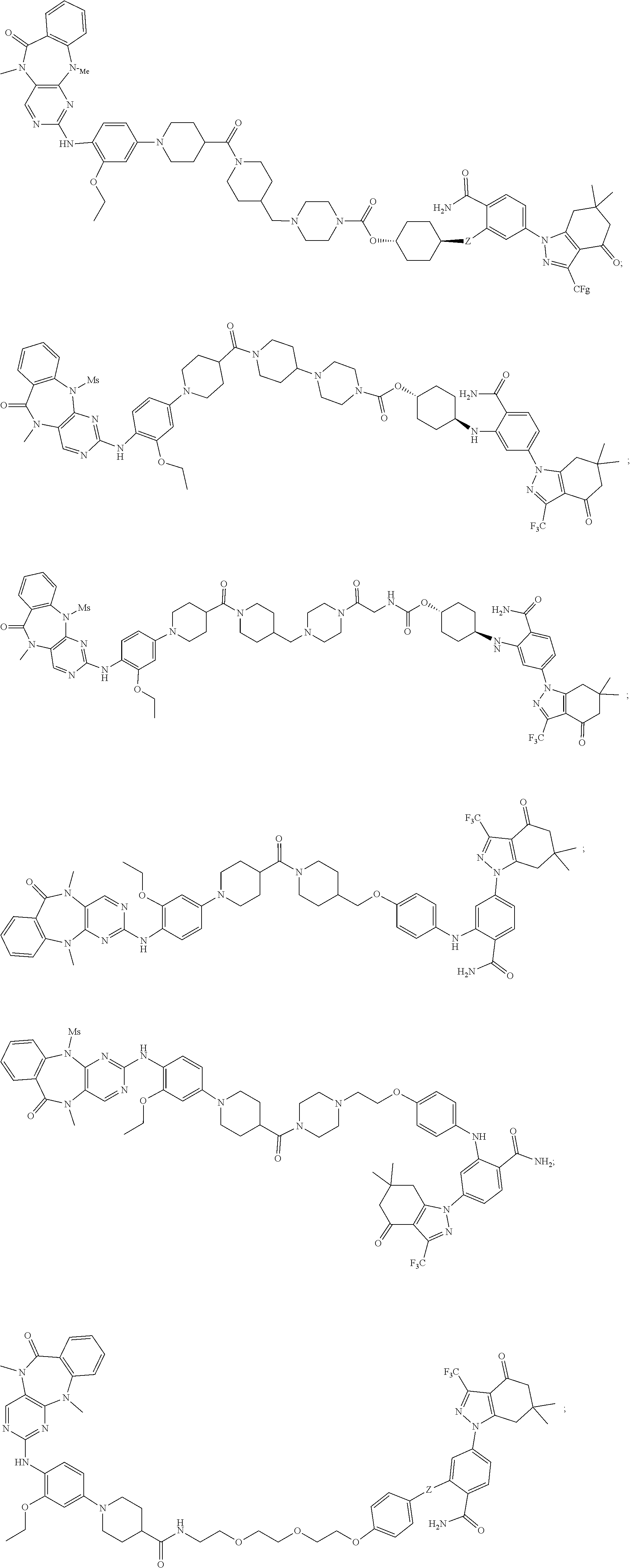 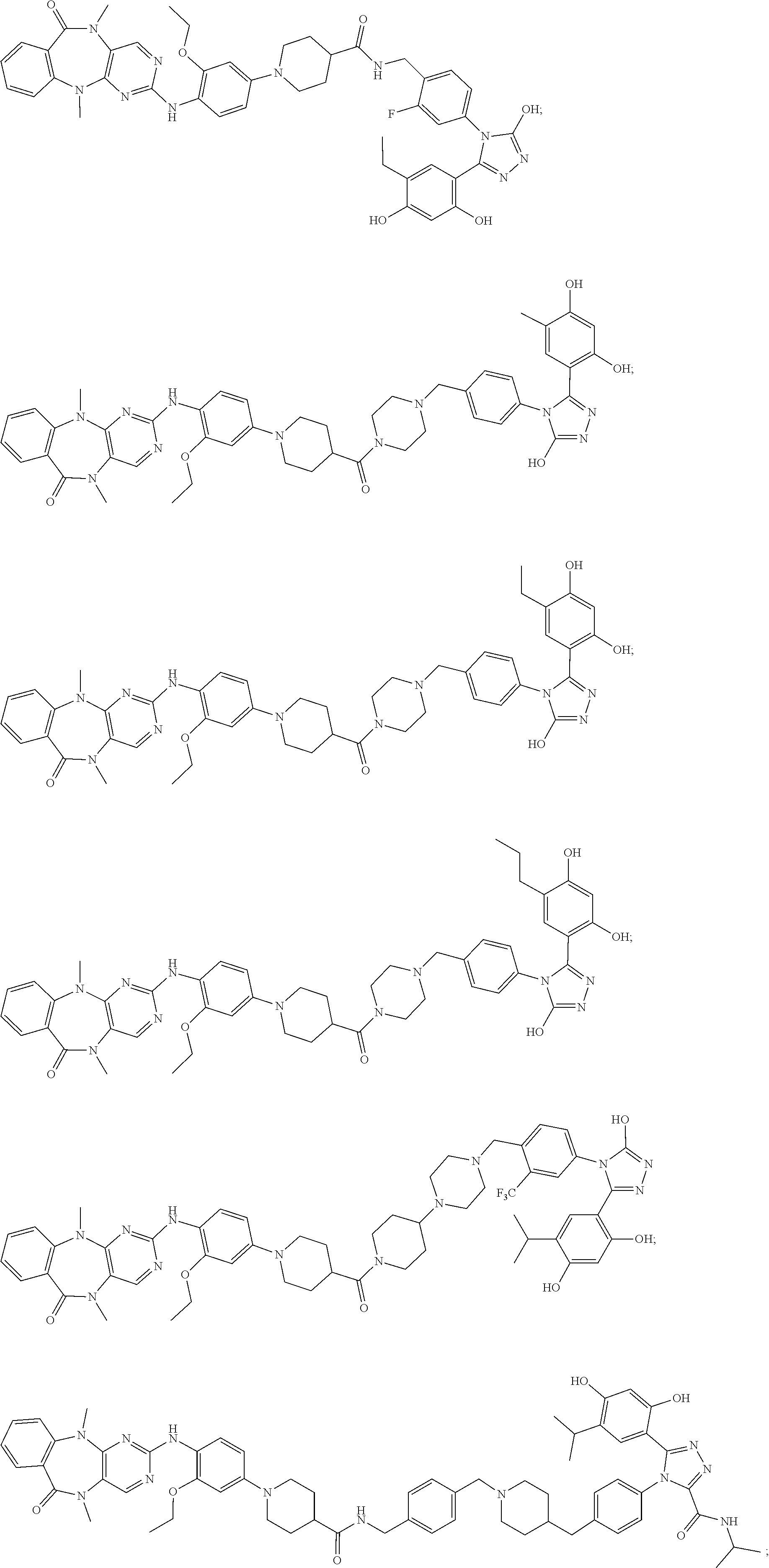 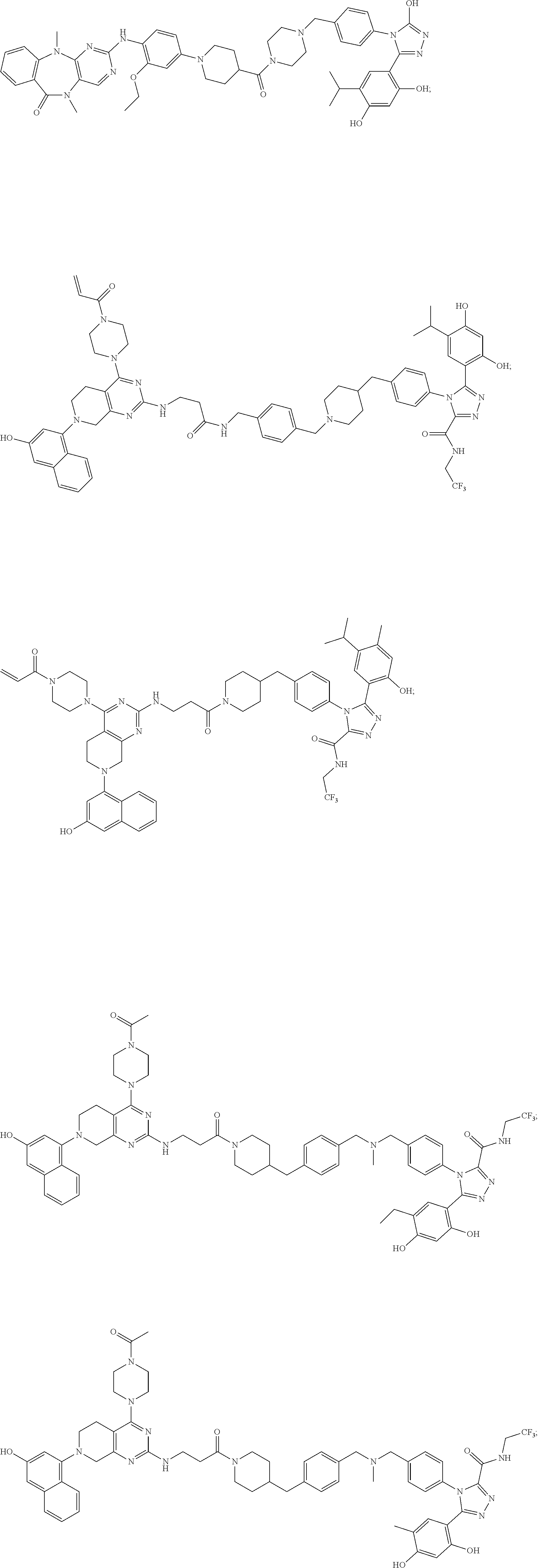 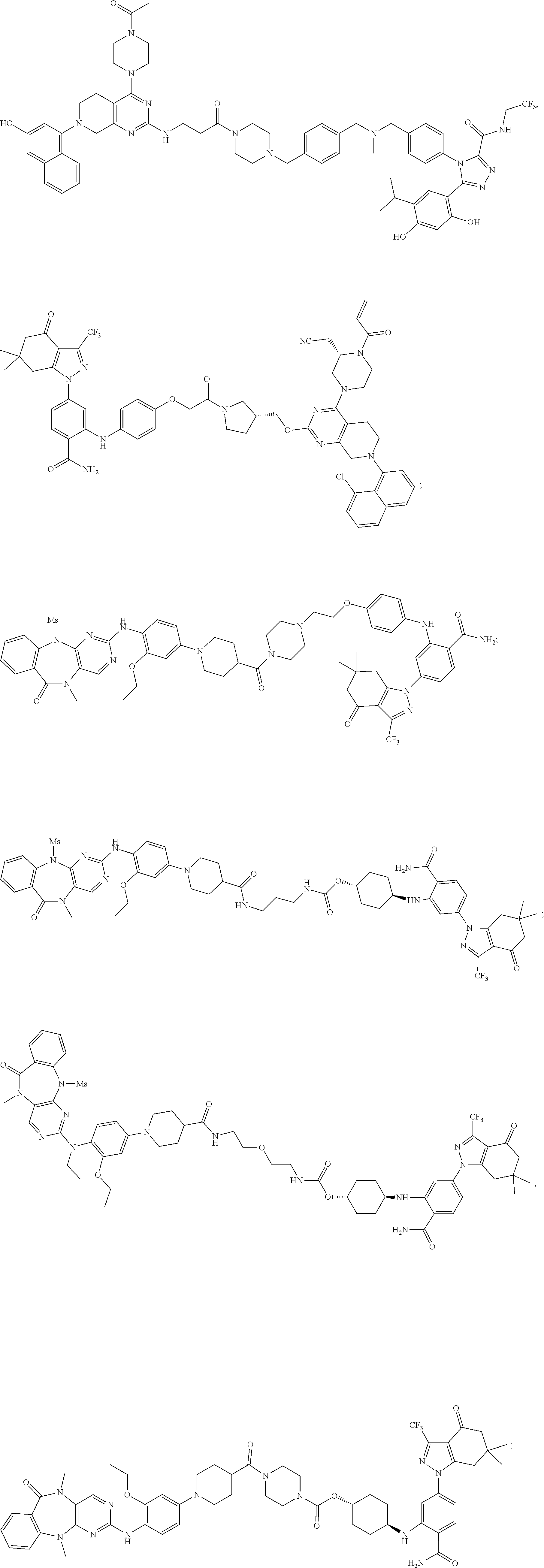 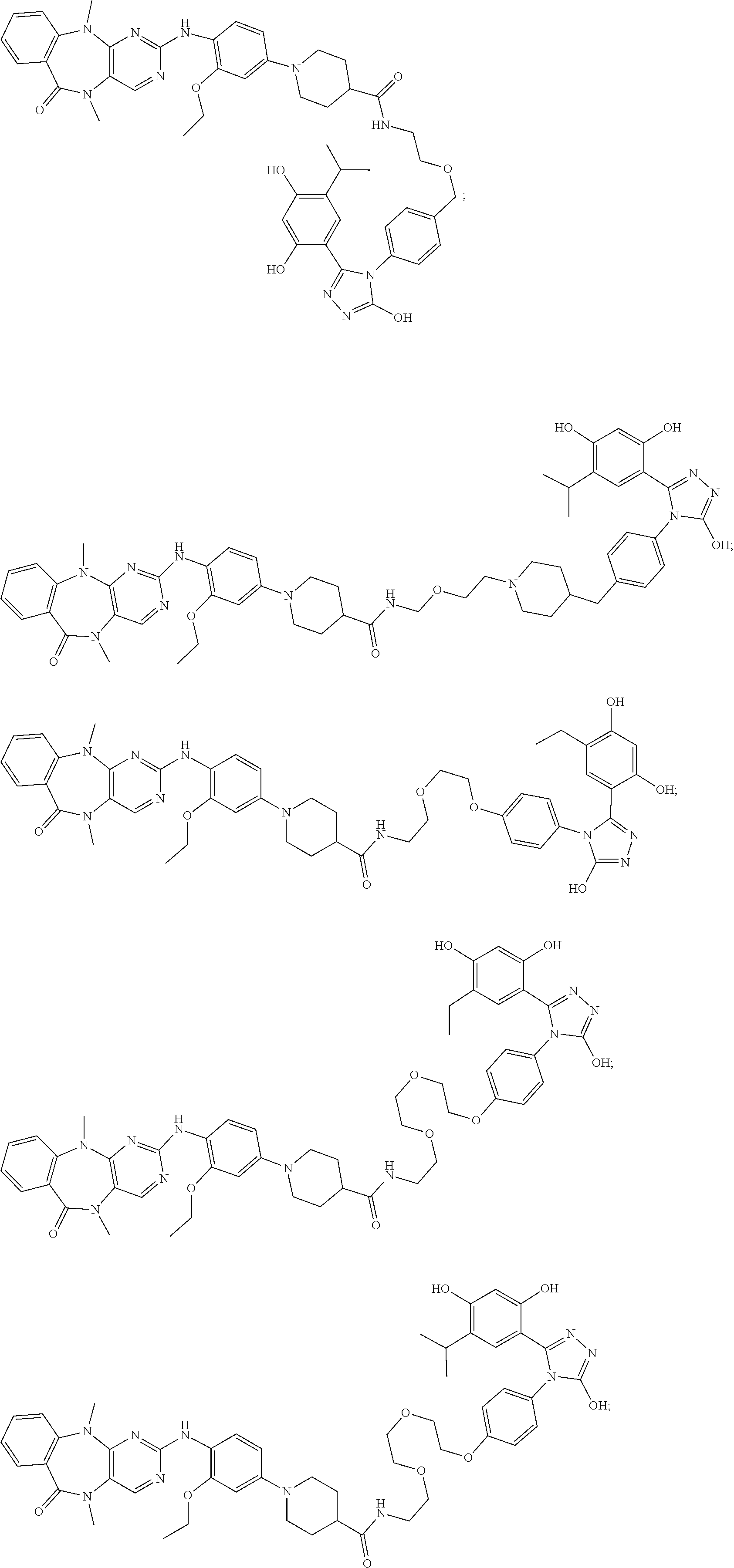 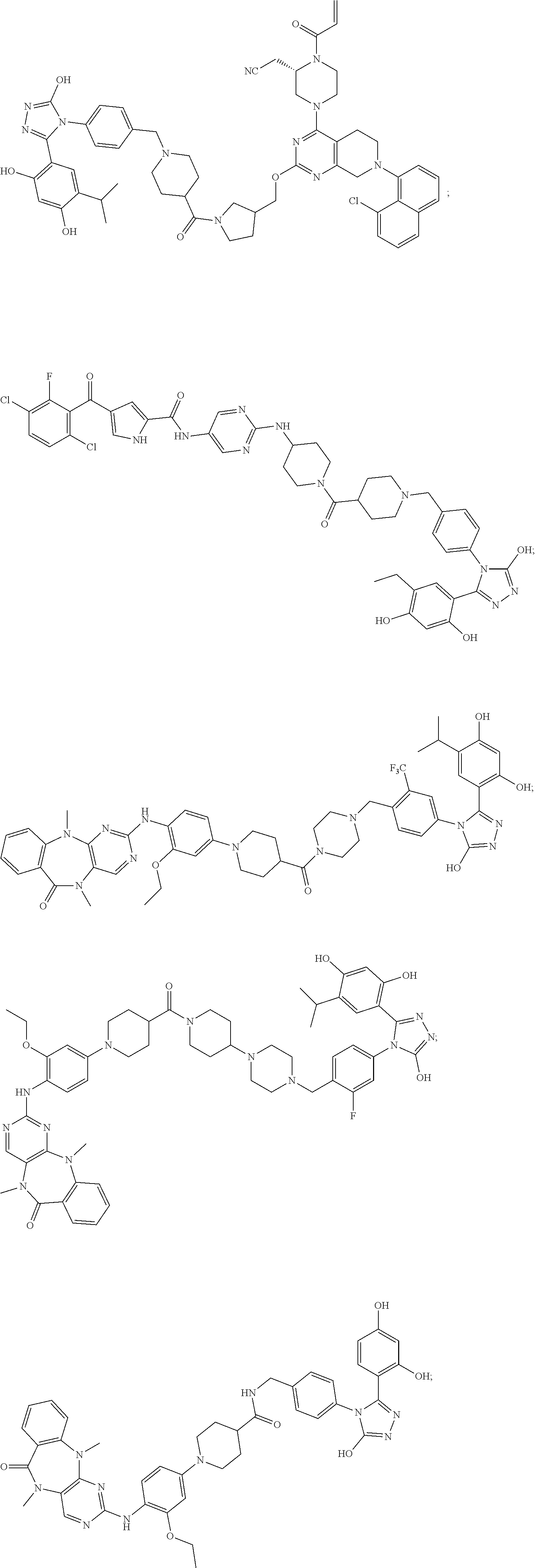 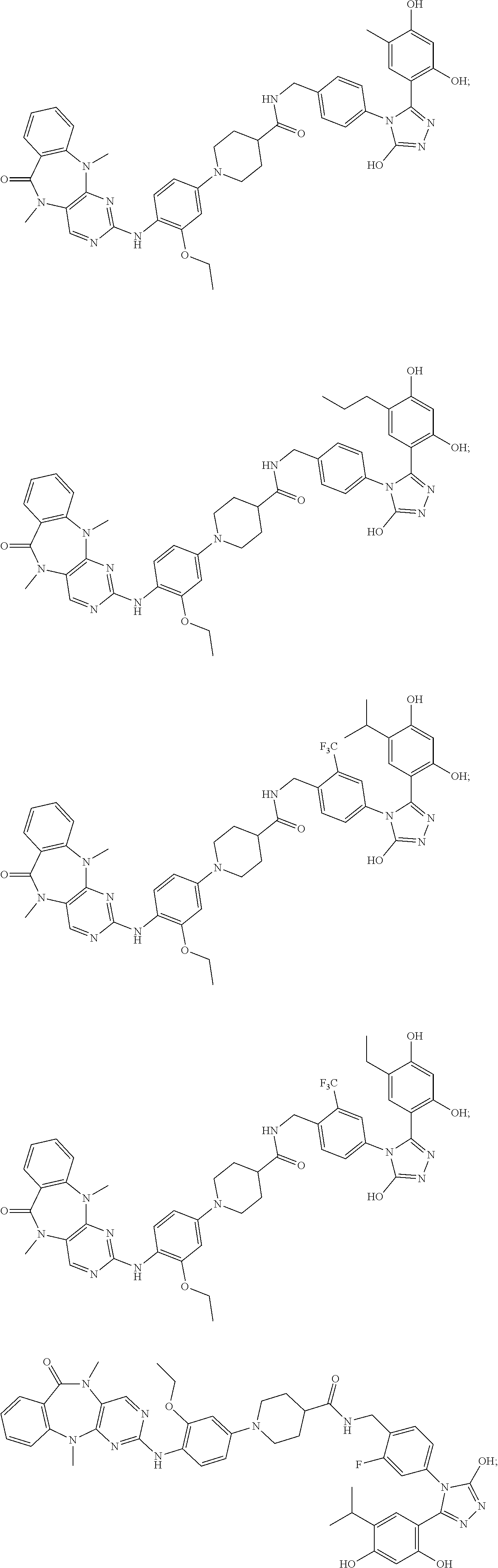
or a pharmaceutically acceptable salt of any of the foregoing.

52. A pharmaceutical composition comprising the compound of claim 1, or a pharmaceutically acceptable salt thereof; and a pharmaceutically acceptable carrier.

53. A method of treating cancer comprising administering to a subject a therapeutically effective amount of a compound of claim 1, or a pharmaceutically acceptable salt thereof.

54. (canceled)

55. (canceled)
